SEQUENCE LISTING (2 sequences)

SEQ ID NO: 1 - protein, 40 aa, synthetic construct
  REGION 1..40: note = human GLP-1 modified at its C-terminus
HHGEGTFTSDLSKQMEEEAVRLFIEWLKNGGPSSPAPPPS

SEQ ID NO: 2 - protein, 30 aa, synthetic construct
  REGION 1..30: note = human glucagon modified at its C-terminus
HHSQGFTFSDYSKYLDSRRAQDFVQWLMNT

(19) United States
(12) Patent Application Publication
(10) Pub. No.: US 2024/0408215 A1
(43) Pub. Date: Dec. 12, 2024

(54) USE OF DIAZIRINE LINKER FOR DRUG CONJUGATES

(71) Applicant: HELMHOLTZ ZENTRUM MUNCHEN - DEUTSCHES FORSCHUNGSZENTRUM FUR GESUNDHEIT UND UMWELT (GMBH), Neuherberg (DE)

(72) Inventors: Alexandre Vieira SILVA, Hannover (DE); Oliver PLETTENBURG, Hannover (DE)

(73) Assignee: HELMHOLTZ ZENTRUM MUNCHEN - DEUTSCHES FORSCHUNGSZENTRUM FUR GESUNDHEIT UND UMWELT (GMBH), Neuherberg (DE)

(21) Appl. No.: 18/690,050

(22) PCT Filed: Sep. 8, 2022
(86) PCT No.: PCT/EP2022/075009

§ 371 Date: Mar. 7, 2024

(30) Foreign Application Priority Data
Sep. 8, 2021 (EP) 21195440.9

(51) Int. Cl. A61K 47/54; A61K 41/00; C07D 203/04

(52) U.S. Cl. CPC A61K 47/545; A61K 41/0042; C07D 203/04

(57) ABSTRACT

The present invention refers to novel UV-light cleavable drug conjugates comprising a diazirine linker.

### BACKGROUND

The development of methods for on-demand drug release is a topic of intense research and interest in the pharmaceutical industry. In general, two options to achieve such kind of pulsatile release exist: Disintegration of a polymeric depot, passively releasing the drug substance or photocleavage of a linker system, releasing the caged molecule. The second approach possesses some advantages in controlling the released amount and the liberation time.

Main stimuli use magnetic waves, ultrasound or near infrared light as a trigger, which all suffer from low spatial resolution. If light is used, several UV light cleavable linkers are described in research publications as exploratory drug delivery systems, however they are difficult to prepare, and linkers described are incompatible with biological systems as they produce byproducts, which are toxic and/or photo-toxic. These problems result in a major issue to develop efficient and safe drug delivery systems and limits the field to in-vitro and few in vivo applications in rodents.

The most frequently used linker system contain o-nitrobenzyl groups and coumarins, azo-benzenes and spiropyrans work by shape change, thus being limited to polymeric or micellar/liposomal delivery (Klan et al., Chem Rev 2013, 113, 119-191).

None of these approaches ever made it into clinical trials, the only approach the inventors are aware which entered clinical trials is a microchip based system, employing electricity to rupture a membrane and then dissolve small amounts of solid material from an opened chamber, this was reported in 2012 (Farra, R. et al. First-in-Human Testing of a Wirelessly Controlled Drug Delivery Microchip. Science Translational Medicine 4, doi:ARTN 122ra2110.1126/scitranslmed.3003276 (2012).

WO2018/200462 describes an insulin-coumarin-conjugate comprising a UV light cleavable group linking the insulin to the coumarin.

WO2014/004278 discloses UV light cleavable drug conjugates. However, neither is any of these linkers a diazirine linker WO2017/045891 describes a light sensitive diazirine linker system for storage of fragrance compounds in washing agents.

Suitable polymers and release systems for pharmaceutical purposes are, e.g., described in Sung and Kim “Recent advances in polymeric drug delivery Systems” Biomaterials Research (2020) 24:12; “Applications of Polymers in Drug Delivery”, Editors: Ambikanandan Misra and Aliasgar Shahiwala 2nd Edition 2021; William B. Liechty et al., “Polymers for Drug Delivery Systems”, Annu. Rev. Chem. Biomol. Eng. 2010. 1:149-73; and Polymer-drug conjugate therapeutics: advances, insights and prospects Iriny Ekladious et al., Nature Reviews Drug Discovery volume 18, pages 273-294 (2019).

UV light emitting devises are known in the art, e.g. an implantable, wireless blue light emitting diode (peak wavelength: 410 nm) (Zhang et al, Photobiomodulation, Photomedicine, and Laser Surgery Vol. 38 No. 11, 2020, pp) or an implantable, wireless LED (Nakajima et al., Oncotarget, 2018, Vol. 9, No. 28, pp 20048-20057).

Sensors and other devices for measuring dependent variables of interest (such as blood glucose) in a patient are generally described in, e.g. US 2009/0054750; US 2009/0164239; US 2008/0172031; US 2005/0065464.

Linker cleavage is usually not traceless, liberating toxic substances and frequently releasing modified drug material, which would make a new drug approval process necessary.

Stimuli used may not be specific and shielded enough, allowing for erroneous drug liberation e.g. by passing a strong magnetic field. Also, low spatial control will make dosing error-prone. Light triggered release of polymer and micellar drug delivery systems are usually based on destabilization of the micelle or polymer, thus leading to a prolonged, not timely well-defined drug release event.

Antibodies or RNA drugs always require injection for application. Also, pharmaceutically active small molecules will be injected in order to achieve rapid onset of action. This process is cumbersome and usually requires presence of a physician or a nurse. The presented approach allows replacing the injection by a remote signal to liberate a given drug molecule from a depot.

Thus, there is a need for drug conjugates to immobilize drugs and allow a controlled release of said immobilized drugs by a trigger.

A new approach for controlled drug release based on a new UV light cleavable linker, which can release (uncage) drugs even in a traceless fashion was surprisingly found.

Especially the latter ensures administration of already registered drugs.

It is compatible with biological systems and even permits the development of “light-activated pills” that can release drugs upon a remote signal, making compounds bioavailable without the need of an injection.

### SUMMARY

Therefore, a first aspect refers to a drug conjugate of formula (A1)

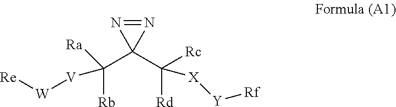

wherein Ra, Rb, Rc, Rd, V, W, X, Y, Re and Rf are as defined in the detailed description below.

In one preferred embodiment in regard of aspect 1 and its other preferred embodiments refers to a drug conjugate of formula (A1), wherein Rc and Rd are each H.

In another preferred embodiment in regard of aspect 1 and its other preferred embodiments, the drug conjugate is a conjugate of Formula (A2)

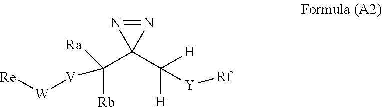

wherein Re, Rf, W, V, Y, Ra and Rb are as defined in Formula (A1).

A second aspect refers to a drug conjugate of formula (A1′)

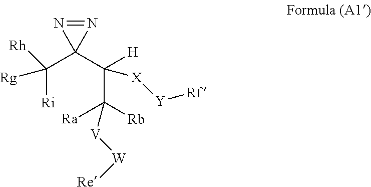

wherein V, W, X, Y, Ra and Rb are as defined for aspect 1 and its preferred embodiments;
Rh, Rg and Ri each individually represent halogen, preferably F, I or Cl, (C₁-C₅)alkyl, phenyl, benzyl; or
Rh, Rg and Ri form together with the carbon to which they are attached an adamantly moiety;
Re′ and Rf′ are identical to Re and Rf as defined for formula (A1) with the exception that an optionally further modified Re′ refers to a modification of Re′ in a conjugate of formula (A1′) with at least one further linker/Rf′ residue of formula (B′) and an optionally further modified Rf′ refers to a modification of Rf′ in a conjugate of formula (A1′) with at least one further linker/Re′ residue of formula (C′):
optionally, Re′ in Formula (A1′) may be further modified by at least one further substitution of a further reactive group of such a drug (preferably, wherein said reactive group is selected from the group consisting of —OH, —SH, —NH₂, —C(O)OH, —C(O)NH₂, —S(O₂)OH, cyclic amides containing an amide group (L₁)-NH—C(O)-(L₂) wherein L₁ and L₂ are carbons of the lactam cycle and a water soluble salt of any of the foregoing), by a linker/Rf′ residue of formula (B′)

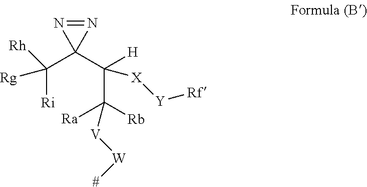

wherein # represents the bond to the further modified Re′; and/or
Rf′ may be further modified by at least one further substitution of a further reactive group of such a drug, (preferably, wherein said reactive group is selected from the group consisting of —OH, —SH, —NH₂, —C(O)OH, —C(O)NH₂, —S(O₂)OH, cyclic amides containing an amide group L₁-N(H)—C(O)-L₂ wherein L₁ and L₂ are carbons of the lactam cycle and a water soluble salt of any of the foregoing), by a linker/Re′ residue of formula (C′)

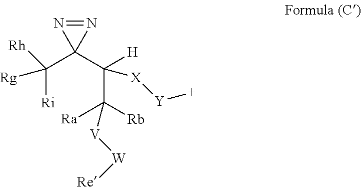

wherein + represents the bond to the further modified Rf′.

The unifying feature of the two aspects is the presence of the diazirine linker in the drug conjugates. This is even more obvious for those embodiments, wherein only one neighbored carbon atom to the diazirine group comprises at least one, more preferably two hydrogen-substituents.

In one preferred embodiment of aspect 1 or 2 or any of their other preferred embodiments, Ra and Rb represent independently from each other a moiety selected from F, Cl, I or Br, (C₁-C₅)alkyl, (C₂-C₅)alkenyl, a five- or six-membered heterocycle, cyclopropyl, cyclopentyl, cyclohexanyl, phenyl, benzyl (—CH₂—C₆H₅); or Ra and Rb form together with the carbon they are attached to a five- or six-membered heterocycle or (C₃₋₁₂)cycloalkyl; or Ra and Rb form together with the carbon they are attached to and V adamantyl (C₁₀H₁₅), iceanyl (C₁₂H₁₇), or diadamantanyl (C₁₄H₁₉).

In one preferred embodiment of aspect 1 or 2 or any of their other preferred embodiments, Re (in aspect 1) or Re′ (in aspect 2) is a modified polymer.

In one preferred embodiment of aspect 1 or any of its other preferred embodiments, Re is further modified by at least one further substitution of a further reactive group of said polymer by a linker/Rf residue of formula (B)

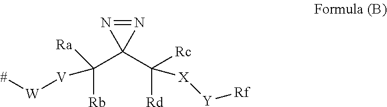

wherein # represents the bond to the further modified Re; or

In one preferred embodiment of aspect 2 or any of its other preferred embodiments, Re′ is further modified by at least one further substitution of a further reactive group of said polymer by a linker/Rf′ residue of formula (B′)

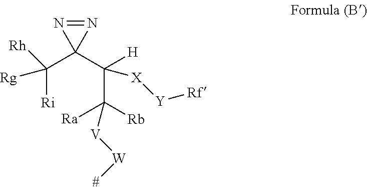

wherein # represents the bond to the further modified Re′.

In one preferred embodiment of aspect 1 or 2 or any of their other preferred embodiments, Re (in aspect 1) or Re′ (in aspect 2) is a modified polymer selected from the group consisting of polyacrylates (PA) (e.g., poly(hydroxyethylmethacrylate)), polymethacrylates (e.g. poly(hydroxyethylmethacrylate) (PHEMA)), polyacrylamides (e.g. poly(N-(2-hydroxypropyl)acrylamide)), polymethacrylamides (e.g. poly[N-(2-hydroxypropyl)]methacrylamide) (HPMA)), poly(alpha-hydroxy acids), poly(lactide-co-glycolides) (PLGA), polylactides (PLA), polyglycolides (PG), functionalized polystyrenes, polyethylene glycol (PEG), poly(alpha-hydroxy acids), polyorthoesters (POE), N-vinyl pyrrolidone, polyaspirins, polyphosphagenes, dendrimers, polyamides, proteins or peptides (e.g. albumin, collagen or fibrin), polysaccharides (e.g. cyclodextrine, agarose, starch, hyaluronic acid, chitosan, gelatin, or alginates) (in one more preferred embodiment selected from the group consisting of polyacrylates, polymethacrylamides, functionalized polystyrenes and polyamides; such as PHEMA or HPMA; in another preferred embodiment selected from the group consisting of poly(alpha-hydroxy acids), PLGA, PLA, PG, PEG, poly(alpha-hydroxy acids), POE, dendrimers, polyaspirins, polyphosphagenes, dendrimers

In one preferred embodiment of aspect 1 or 2 or any of their other preferred embodiments, V and X represent independently from each other a bond, —CH₂—, —CH₂—CH₂—, or —CH₂—CH₂—CH₂—.

In one preferred embodiment of aspect 1 or 2 or any of their other preferred embodiments, X represents a bond.

In one preferred embodiment of aspect 1 or 2 or any of their other preferred embodiments, W and Y independently from each other represents a moiety selected from the group consisting of *—O—, *—S—, *—NH—, *—C(O)—O—, *—O—C(O)—, *—C(O)—NH—, *—NH—C(O)—, *-triazol-, —O—(C₂-C₅)alkyl-triazol-, *—O—C(O)—(C₂-C₅)alkyl-triazol-, *—C(O)O—(C₂-C₅)alkyl-triazol-.

In a further preferred embodiment of aspect 1 or 2 or any of their other preferred embodiments, W and Y each represent a different moiety.

In a further preferred embodiment of aspect 1 or 2 or any of their other preferred embodiments, Rf (in aspect 1) or Rf (in aspect 2) represents a drug selected from the group consisting of ACE-inhibitors; anti-anginal drugs; anti-arrhythmias; anti-asthmatics; anti-cholesterolemics; anti-convulsants; anti-depressants; anti-diarrhea preparations; anti-histamines; anti-hypertensive drugs; anti-infectives; anti-inflammatory agents; anti-lipid agents; anti-manics; anti-nauseants; anti-stroke agents; anti-thyroid preparations; anti-tumor drugs; anti-tussives; anti-uricemic drugs; anti-viral agents; acne drugs; alkaloids; amino acid preparations; anabolic drugs; analgesics; anesthetics; angiogenesis inhibitors; antacids; anti-arthritics; antibiotics; anticoagulants; antiemetics; antiobesity drugs; antiparasitics; antipsychotics; antipyretics; antispasmodics; antithrombotic drugs; anxiolytic agents; appetite stimulants; appetite suppressants; beta blocking agents; bronchodilators; cardiovascular agents; cerebral dilators; chelating agents; cholecystokinin antagonists; chemotherapeutic agents; cognition activators; contraceptives; coronary dilators; cough suppressants; decongestants; deodorants; dermatological agents; diabetes agents; diuretics; emollients; enzymes; erythropoietic drugs; expectorants; fertility agents; fungicides; gastrointestinal agents; growth regulators; hormone replacement agents; hyperglycemic agents; hypnotics; hypoglycemic agents; laxatives; migraine treatments; mineral supplements; mucolytics; narcotics; neuroleptics; neuromuscular drugs; NSAIDS; nutritional additives; peripheral vasodilators; peptides; prostaglandins; psychotropics; renin inhibitors; respiratory stimulants; steroids; stimulants; sympatholytics; thyroid preparations; tranquilizers; uterine relaxants; vaginal preparations; vasoconstrictors; vasodilators; vertigo agents; vitamins; wound healing agents; and contrast agents.

In a further preferred embodiment of aspect 1 or 2 or any of their other preferred embodiments, said drug is a therapeutic peptide, preferably selected from the group consisting of Glucagon and its derivatives, GLP-1 and its derivatives specifically exendin, lixisenatide, liraglutide and semaglutide, Insulin and its derivatives, specifically the short acting insulin derivatives insulin glulisine, insulin aspart and insulin lispro and Somatostatin analogues specifically lanreotide, more preferably insulin, more preferably wherein said therapeutic peptide is insulin in a hexameric form combined with zinc.

In a further preferred embodiment of aspect 1 or 2 or any of their other preferred embodiments, said drug is an emergency drug selected from the group consisting of Epinephrine, Nitroglycerin, Antihistamine (diphenhydramine), Albuterol, Salbutamol, Glucagon, Atropine, Ephedrine, Hydrocortisone, Morphine, Naloxone, Lorazepam, Midazolam, Flumazenil, Streptokinase.

In a further preferred embodiment of aspect 1 or 2 or any of their other preferred embodiments, said drug is a cytotoxic agent selected from the group consisting of Tubulin Inhibitor (preferably Metransine; Maytansine derivatives such as vinblastine, vinorelbine, vindesine and ravtansine; Taxanes such as paclitaxel, docetaxel, epothilone derivatives like ixabepilone, patupilone and utidelone; Cholchicine and cholchicin derivatives), Heat Shock Protein 90 inhibitors (HSP90) (preferably radicicol, geldanamycin and 17-N-Allylamino-17-demethoxygeldanamycin), Topoisomerase Inhibitors (preferably camptothecin and its analogues such as Atiratecan, Topotecan, Exatecan, Pegamotecan, Protecan and Irinotecan), Calicheamicin and derivatives (preferably n-acetyl-γ-calicheamicin), Kinase inhibitors (preferably Monomethyl auristatin and Auristatin F-hydroxypropylamide), DNA minor groove binding alkylating agents (preferably duocarmycin and its derivatives (A, B1, B2, C1, C2, DA)CC-1065, adozelesin, bizelesin, and carzelesin), DNA intercalators (preferably doxorubicin), 5-fluorouracil and derivatives (preferably Capecitabine and Tegafur), Temozolomide, Lenalidomide, and Pomalidomide.

In a further preferred embodiment of aspect 1 or 2 or any of their other preferred embodiments, said drug is an antibody (preferably Infliximab, Adalimumab, Ustekinumab, Omalizumab, Leronlimab, Dupilumab, Brolucizumab, Eptinezumab, Teprotumumab, Crizanlizumab, Satralizumab, Sarilumab, Tanezumab (e.g., suitable for immune and inflammatory disorders), Nivolumab, Pembrolizumab, Trastuzumab, Bevacizumab, Rituximab, Dostarlimab, Spartalizumab, Ublituximab (e.g. suitable for cancer therapy), Panitumumab, CanacinumabGolimumab, Ofatumumab, Denosumab, Belimumab, Ipilimumab, Ramucirumab, Nivolumab, Alirocumab, Daratumumab, Necitumumab, Evolocumab, Sekukinumab, Olaratumab, Atezulizumab, Avelumab, Brodalumab, Dupilumab, Durvalumab, Guselkumab, Sarilumab, Erenumab, Cemiplimab, Emapalumab, Maxetumomab pasodudax, more preferably Infliximab, adalimumab, ustekinumab, omalizumab, leronlimab, dupilumab, brolucizumab, eptinezumab, teprotumumab, crizanlizumab, satralizumab, sarilumab, tanezumab, Nivolumab, Pembrolizumab, Trastuzumab, Bevacizumab, Rituximab, Dostarlimab, Spartalizumab, Ublituximab).

A third aspect refers to the use of a diazirine group for releasing an immobilized drug from a drug conjugate by applying UV light to the diazirine group.

A fourth aspect refers to a linker molecule of formula (A′)

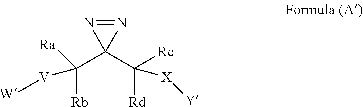

wherein
Ra and Rb independently from each other represents a moiety selected from H, halogen (preferably F, Cl, I or Br), (C₁-C₅)alkyl, (C₂-C₅)alkenyl, a five- or six-membered heterocycle, (C₃₋₃₀)cycloalkyl (preferably cyclopropyl, cyclopentyl, cyclohexanyl, adamantyl (C₁₀H₁₅), iceanyl (C₁₂H₁₇), diadamantanyl (C₁₄H₁₉), triadamantyl (C₂₂H₂₃), isotetramantanyl (C₂₂H₂₇), pentamantanyl (C₂₆H₃₁), cyclohexamantanyl (C₂₆H₂₉), superadamantan (C₃₀H₃₅)), phenyl, benzyl (—CH₂—C₆H₅) each heterocycle, cycloalkyl, phenyl or benzyl can optionally be substituted with 1, 2, 3, 4 or 5 substituents selected from the group consisting of hydroxy, halogen, (C₁—C₅)alkyl, (C₁-C₅)alkoxy, more preferably selected from halogen (preferably F, Cl, I or Br), (C₁-C₅)alkyl, (C₂-C₅)alkenyl, a five- or six-membered heterocycle, (C₃₋₃₀)cycloalkyl (preferably cyclopropyl, cyclopentyl, cyclohexanyl, adamantyl (C₁₀H₁₅), iceanyl (C₁₂H₁₇), diadamantanyl (C₁₄H₁₉), triadamantyl (C₂₂H₂₃), isotetramantanyl (C₂₂H₂₇), pentamantanyl (C₂₆H₃₁), cyclohexamantanyl (C₂₆H₂₉), superadamantan (C₃₀H₃₅)), phenyl, benzyl (—CH₂—C₆H₅) each heterocycle, cycloalkyl, phenyl or benzyl can optionally be substituted with 1, 2, 3, 4 or 5 substituents selected from the group consisting of hydroxy, halogen, (C₁-C₅)alkyl, (C₁-C₅)alkoxy; or
Ra and Rb form together with the carbon they are attached to a five- or six-membered heterocycle or (C₃₋₁₂)cycloalkyl, preferably cyclopentyl or cyclohexyl optionally substituted with 1, 2, 3, 4 or 5 substituents selected from the group consisting of hydroxy, halogen, (C₁-C₅)alkyl, (C₁-C₅)alkoxy); or
Ra and Rb represent together with the carbon they are attached to and V a (C₃₋₃₀)cycloalkyl moiety to which —W—Re is attached wherein the cycloalkyl moiety is selected from the group consisting of adamantyl (C₁₀H₁₄), iceanyl (C₁₂H₁₆), diadamantanyl (C₁₄H₁₈), triadamantyl (C₂₂H₂₂), isotetramantanyl (C₂₂H₂₆), pentamantanyl (C₂₆H₃₀), cyclohexamantanyl (C₂₆H₂₈), superadamantan (C₃₀H₃₄), each optionally substituted with 1, 2, 3, 4 or 5 substituents selected from the group consisting of hydroxy, halogen, (C₁-C₅)alkyl, (C₁-C₅)alkoxy; more preferably the cycloalkyl is selected from the group consisting of adamantyl (C₁₀H₁₄), iceanyl (C₁₂H₁₆), diadamantanyl (C₁₄H₁₈), triadamantyl (C₂₂H₂₂), isotetramantanyl (C₂₂H₂₆), pentamantanyl (C₂₆H₃₀), cyclohexamantanyl (C₂₆H₂₈), superadamantan (C₃₀H₃₄), more preferably the cycloalkyl is selected from the group consisting of adamantyl (C₁₀H₁₄), iceanyl (C₁₂H₁₆), diadamantanyl (C₁₄H₁₈), even more preferably adamantyl (C₁₀H₁₄)); or
Ra and Rb form together with the carbon they are attached to and V a phenyl moiety to which —W′ is attached;
Rc or Rd in formula represent H, the other space holder Rc or Rd, respectively, V and X are as defined in claims 1, 2 and 7 for the drug conjugate of formula (A1) and W′ and Y′ are independently selected from a group consisting of hydroxy (—OH), thiol (—SH), amin (—NH₂), —Cl, —Br, —F or —I, a cyclic amide function (—N(H)—C(O)—) such as in a lactame, wherein the free bond of the nitrogen and the free bond of the C(O) carbon each represents a bond to a neighbored carbon atom of the cycle, sulfonic acid (—S(O₂)OH), —O—S(O₂)OH, thiocarboxylic acid (—C(S)OH), carboxyl (carboxylic acid (—C(O)OH)), a water soluble salt, such as a pharmaceutically acceptable salt, thereof, a carboxylic acid halide and anhydrates thereof), amide (—C(O)NH₂), carboxylic acid ester, preferably with (C₁-C₅)alkyl, isothiocyanate (—NCS), isocyanate (—NCO) such as —(C₁-C₅)—NCO, —NCO, —O—(C₁-C₅)—NCO, —O—NCO, —O—C(O)—(C₁-C₅)—NCO, —O—C(O)—NCO or —C(O)O—(C₁-C₅)NCO, —C(O)O—NCO hydrazine, a water soluble salt, preferably a pharmaceutically acceptable salt, of any of the foregoing, alkyne such as —O—(C₁-C₅)alkyl-ethynyl, —C(O)O—(C₁-C₅)alkyl-ethynyl or —O—C(O)—(C₁-C₅)alkyl-ethynyl, azide, such as —(C₁-C₅)alkyl-N₃, —N₃, —O—(C₁-C₅)alkyl-N₃, —O—C(O)—(C₁-C₅)alkyl-N₃ or —C(O)O—(C₁-C₅)alkyl-N₃, maleimide, imidate, alkenyl such as —O—(C₃—C)-alkenyl, —C(O)—O—(C₃-C₇)-alkenyl, —O—C(O)—(C₃—C)-alkenyl, a conjugated diene such as a conjugated —(C₄-C₁₀)alkdienyl, a conjugated —O—(C₅-C₁₀)alkdienyl, a conjugated —O—C(O)—(C₅-C₁₀)alkdienyl or —C(O)O—(C₅C₁₀)alkdienyl, tetrazinyl such as —(C₁-C₅)alkyl-tetrazinyl, -tetrazinyl, —O—(C₁-C₅)tetrazinyl, —O-tetrazinyl, —O—C(O)—(C₁-C₅)tetrazinyl, —O—C(O)-tetrazinyl, —C(O)O-tetrazinyl or —C(O)O—(C₁-C₅)tetrazinyl.

A fifth aspect refers to a depot suitable for implantation into a patient comprising at least one drug conjugate according to aspect 1 or 2 or any of their preferred embodiments.

In one preferred embodiment, the depot further comprises a UV-light source device and a regulator device for the UV-light source device, wherein the regulator is wirelessly connected to a control device and the UV-light source device can be activated, regulated and deactivated by the control device via the wireless connection between the control device and the regulator device.

A sixth aspect refers to a method of administering a drug to a patient comprising: implanting a drug conjugate according to aspect 1 or 2 or any of their preferred embodiments or a depot according to aspect 6 and any of its preferred embodiments into a patient;
activating a UV light source to emit light with a wave length and intensity sufficient to cleave at least part of the linker of a conjugate according to aspect 1 or 2 or any of their preferred embodiments thereby releasing the drug from the drug conjugate.

### Definitions

The person skilled in the art is aware that the expressions “a” or “an” as used in the present application may, depending on the situation, mean “one (1)”, “one (1) or more” or “at least one (1)”.

When amino acid sequences are disclosed in this application, the amino acids are usually indicated in form of their IUPAC amino acid code or the three letter code:

| IUPAC amino acid code | Three letter code | Amino acid |
|---|---|---|
| A | Ala | Alanine |
| C | Cys | Cysteine |
| D | Asp | Aspartic Acid |
| E | Glu | Glutamic Acid |
| F | Phe | Phenylalanine |
| G | Gly | Glycine |
| H | His | Histidine |
| I | Ile | Isoleucine |
| K | Lys | Lysine |
| L | Leu | Leucine |
| M | Met | Methionine |
| N | Asn | Asparagine |
| P | Pro | Proline |
| Q | Gln | Glutamine |
| R | Arg | Arginine |
| S | Ser | Serine |
| T | Thr | Threonine |
| V | Val | Valine |
| W | Trp | Tryptophan |
| Y | Tyr | Tyrosine |

The terms “(Cₐ-C_{b})” “(C_{a-b})” and “Cₐ-C_{b}-”, wherein a and b represent integer, are exchangeable and relate to the minimum and maximum number of carbon atoms in an organic group. “(C₁-C₅)” “(C₁₋₅)” and “C₁-C₅-” alkyl, for example, relate to an alkyl group having 1, 2, 3, 4 or 5 carbon atoms. The expressions “(Cₐ)” and “Cₐ—” are likewise exchangeable and relate to the number of carbon atoms in an organic group. The expressions “C₃-cycloalkyl” and “(C₃)-cycloalkyl”, for example, relate to cyclopropyl.

The term “alkyl”—on its own or as a part of a chemical group—represents straight-chain or branched hydrocarbons having Cₐ-C_{b} carbon atoms as mentioned below, for example the term (C₁-C₅)alkyl includes methyl, ethyl, n-propyl, isopropyl, n-butyl, isobutyl, s-butyl, t-butyl, n-pentyl, 2-pentyl, 3-pentyl, 2-methylbutyl, 3-methylbutyl, 2-methylbut-2-yl. 2,2-dimethylpropyl. The alkyl radicals according to the invention may optionally be substituted.

The term “cycloalkyl”—on its own or as part of a chemical group—represents mono-, bi- or tri or polycyclic hydrocarbons, including diamondoids, preferably having 3 to 30 carbons, for example cyclopropyl, cyclobutyl, cyclopentyl, cyclohexyl, bicyclo[2.2.1]heptyl, bicyclo[2.2.2]octyl or adamantly. The cycloalkyl radicals according to the invention may optionally be substituted.

The term “alkoxy” is known to the skilled person. It refers to —O—(Cₐ-C_{b})alkyl residues. The alkoxy radicals according to the invention may optionally be substituted.

The term “linker molecule” as used herein refers to a molecule comprising a diazirine group (linker) and two reactive groups selected from the group consisting of hydroxy (—OH), thiol (—SH), amin (such as —NH₂ or N((C₁-C₅)alkyl)H), sulfonic acid (—S(O₂)OH), —O—S(O₂)OH, thiocarboxylic acid (—C(S)OH), carboxyl (such as carboxylic acid (—C(O)OH), amide (—C(O)NH₂), carboxylic halide such as —C(O)Cl, carboxylic acid ester such as an ester with (C₁-C₅)alkyl, carboxylic acid anhydride; the carboxyl group may be activated, as is well known in the art, to facilitate coupling), isothiocyanate (—NCS), isocyanate (—NCO), hydrazine, a water soluble salt of any of the foregoing, alkyne such as —O—(C₁-C₅)alkyl-ethynyl, —C(O)O—(C₁-C₅)alkyl-ethynyl or —O—C(O)—(C₁-C₅)alkyl-ethynyl, azide, such as —O—(C₁-C₅)alkyl-N₃, —O—C(O)—(C₁-C₅)alkyl-N₃ or —C(O)O—(C₁-C₅)alkyl-N₃, maleimide, imidate, if appropriate, water soluble salts of these groups or a combination of two of these groups which are suitable to form a drug conjugate of formula (A1) as defined herein, by forming a bond to a drug or polymer via a reaction of one of the reactive groups of the linker molecule with a reactive group of a drug or a polymer as defined herein. For example, a carboxylic acid group of a linker molecule can react with a hydroxy group of a polymer to form an ester. Exemplary linker molecules are described herein, e.g. those of formula (A′).

The term “linker” as used herein refers to a UV light cleavable diazirine group. Such a diazirine group which is on the one hand linked via various carbon chains and reactive groups to a polymer or a drug and on the other hand to a drug, thus, linking the polymer and the drug or two drugs, respectively. Preferably, the linker breaks down by applying UV light and all (by)products of such a UV light-initiated break-down are non-toxic and/or non-photo-toxic. The linkers as used herein are part of a construct of a linker molecule with two (optionally independently from each other further modified as defined herein) drug molecules or with one (optionally further modified as defined herein) polymer and one (optionally further modified as defined herein) drug molecule.

The term “biocompatible” as used herein refers to a polymer (and thus the depot) or byproduct (which results from the breakdown of the linker) which will not cause substantial tissue irritation or necrosis at the target tissue site of a mammal. Preferably, the polymer is approved for use in the body by the Food and Drug Administration.

The term “biodegradable” as used herein generally refers to a base polymer or byproduct (which results from the breakdown of the linker) that breaks down into oligomeric and/or monomeric units over a period of time, typically over days, weeks, or even months, when implanted or injected into the body of a mammal. Typically, the term “biodegradable” includes that all or parts of the drug depot will degrade over time by the action of enzymes, by hydrolytic action and/or by other similar mechanisms in the human body. “Biodegradable” generally means that the depot can break down or degrade within the body to non-toxic components after or while the drug has been or is being released.

The term “bioresorbable” as used herein refers to a polymer or byproduct whose degradative products are metabolized in vivo or excreted from the body via natural pathways. In general, by “bioabsorbable,” it is meant that the depot will be broken down and absorbed within the human body, for example, by a cell or tissue.

The term “click chemistry” or “click reaction” or (click chemistry reaction” as used herein can be used interchangeably. The skilled person is aware of general click reactions. A click reaction is not disturbed by water, generate minimal and inoffensive byproducts and fulfills certain characteristics such as modularity, insensitivity to solvent parameters, high chemical yields, insensitivity towards oxygen and water, regiospecificity and stereospecificity, a large thermodynamic driving force (>20 kcal/mol) to favor a reaction with a single reaction product. Non-limiting examples for click reactions are [3+2] cycloadditions, such as the Huisgen 1,3-dipolar cycloaddition (reaction between an azide function and an ethynyl function (alkyne)) in particular the Cu(I)-catalyzed stepwise variant; Thiol-ene reaction (reaction between a thiol and an alkene to form a thioether); Diels-Alder reaction and inverse electron demand Diels-Alder reaction (conjugated diene and a substituted alkene resulting in a cyclohexene); [4+1] cycloadditions between isonitriles (isocyanides) and tetrazines resulting in 4-imine-pyrazoles.

The term “depot” as used herein refers to at least one conjugate according to the invention or at least one modified polymer according to the invention, each of them being suitable to be implanted into a patient. Optionally, a depot further comprises a UV light source, such as a LED device, including a power supply for said LED, a regulator device for the light source, e.g., which can be wirelessly (e.g. by radiocommunication) connected with a controller device or is connected with a detector measuring a parameter to be corrected by the release of a drug from the depot, and optionally a biocompatible surface such as a stainless steel form which comprises a membrane which is permeable for a released drug.

The term “drug” as used herein refers to a pharmaceutically acceptable compound which is suitable for therapy (alters the physiology of a patient) or diagnostic, said drug has at least one reactive group or functionality that will allow it to be joined to a UV light cleavable linker molecule as described herein. The term “drug” may be used interchangeably herein or in the art with the terms “biologically active agent,” “therapeutic agent,” and “active pharmaceutical ingredient” or prodrug thereof as known in the art. A drug according to the invention comprises at least one reactive group selected from the group consisting of hydroxy (—OH), thiol (—SH), amin (—NH₂), sulfonic acid (—S(O₂)OH), —O—S(O₂)OH, thiocarboxylic acid (—C(S)OH), carboxyl (such as carboxylic acid (—C(O)OH), amide (—C(O)NH₂), cyclic amides containing an amide group in the cycle (—N(H)—C(O)—), carboxylic halide, preferably —C(O)Cl, carboxylic acid ester, preferably with (C₁-C₅)alkyl, or carboxylic acid anhydride; the carboxyl group may be activated, as is well known in the art, to facilitate coupling), isothiocyanate (—NCS), isocyanate (—NCO), hydrazine, a water soluble salt of any of the foregoing, alkyne, preferably —O—(C₁-C₅)alkyl-ethynyl, —C(O)O—(C₁-C₅)alkyl-ethynyl or —O—C(O)—(C₁-C₅)alkyl-ethynyl, azide, preferably —O—(C₁-C₅)alkyl-N₃, —O—C(O)—(C₁-C₅)alkyl-N₃ or —C(O)O—(C₁-C₅)alkyl-N₃, maleimide, imidate.

The terms “UV” or “UV-light” can be used interchangeably herein and refer to light with a wave-length between 100 and 400 nm.

The term “free form” of a polymer or drug as used herein refers to the structure of such a polymer or drug before the polymer or drug is reacted with a reactive group of a linker molecule, of a linker molecule which is already connected with one of its two reactive groups to a polymer or of a linker molecule which is already connected with one of its two reactive groups to a drug.

Usually, the structure associated with the common name of a drug represents the free form of a drug as used herein. Thus, the term “free form” and “native”, especially in combination with a drug such as a peptide or hormone, can also be used interchangeably herein. For example, if a drug or polymer is connected to a linker and the link to the linker is represented by Y of formula (A1) or (A1′) being *—O—, the free form of the drug or polymer will comprise a restored —OH group at the position of Y when the linker broke down; if a drug or polymer is connected to a linker and the link to the linker is represented by Y of formula (A1) or (A1′) being *—S—, the free form of the drug will comprise a restored —SH group at the position of Y when the linker broke down; if a drug or polymer is connected to a linker and the link to the linker is represented by Y of formula (A1) or (A1′) being *—O—C(O)—, the free form of the drug will comprise a restored —COOH group a pharmaceutically acceptable salt thereof, a water-soluble salt thereof, or an anhydride thereof at the position of Y when the linker broke down; if a drug or polymer is connected to a linker and the link to the linker is represented by Y of formula (A1) or (A1′) being *—NH—, the free form of the drug will comprise a restored —NH₂ group at the position of Y when the linker broke down; if a drug or polymer is connected to a linker and the link to the linker is represented by Y of formula (A1) or (A1′) being *—N(C(O)—)— (e.g. resulting from the reaction of the cyclic amid group of a lactame with an alkyl halide (see, e.g. Example 2d), the free form of the drug will comprise a restored amide group (—N(H)—C(O)—) in a lactame cycle in the free form of Re or Rf, respectively when the linker broke down; if a drug or polymer is connected to a linker and the link to the linker is represented by Y of formula (A1) or (A1′) being *—O—C(O)—NH—, the free form of the drug will comprise a restored —NH₂ group at the position of Y when the linker broke down; if a drug or polymer is connected to a linker and the link to the linker is represented by Y of formula (A1) or (A1′) being *—N((C₁-C₅)alkyl)-, the free form of the drug will comprise a restored —N((C₂-C₅)alkyl)H group (a secondary amine) at the position of Y when the linker broke down; if a drug or polymer is connected to a linker and the link to the linker is represented by Y of formula (A1) or (A1′) being *—NH—C(O)—, the free form of the drug will comprise a restored —CONH₂ group at the position of Y when the linker broke down; if a drug or polymer is connected to a linker and the link to the linker is represented by Y of formula (A1) or (A1′) being *—O—S(O₂)—, the free form of the drug will comprise a restored —S(O₂)OH group a pharmaceutically acceptable salt thereof, a water soluble salt thereof, or an anhydride thereof at the position of Y when the linker broke down.

In addition, the free form also encompasses drugs, especially peptides, which are functionalized in that they comprise a reactive group such as —N₃, -ethynyl, thiol (—SH), ethenyl or a conjugated diene function (—C═C—C═C—), respectively, which result (by reacting with a corresponding click chemistry group (e.g., —N₃ with -ethynyl, -ethenyl with thiol, or ethenyl with a conjugated diene function) of a linker molecule or of a polymer-linker molecule conjugate or of a drug-linker molecule conjugate) via a click chemistry reaction in a respective moiety such as a triazole moiety, a cyclohexenyl moiety or a thioether moiety. The skilled person is aware of further click chemistry educt group pairs. When the linker of a drug conjugate breaks down the released form of the drug is then of course a free form derivative comprising a residue being the result of a click chemistry step.

The term “free form derivative” of a drug as used herein refers to a released drug having a click chemistry resulting group selected from the group consisting of HO—(C₂-C₅)alkyl-triazol-, HO—C(O)—(C₂-C₅)alkyl-triazol-, H—O—(C₂-C₅)alkyl-cyclohexenyl-H—O—C(O)—(C₂-C₅)alkyl-cyclohexenyl(wherein each cyclohexenyl moiety is optionally further substituted with 1, 2 or 3 (C₁-C₄)alkyl), HS—CH₂—CH₂— and HO—CH₂—CH₂—, or a (pharmaceutically acceptable) salt of any of the foregoing.

Notably the free form derivative should have the same pharmaceutically activity as the free form. The level of said activity may be the same compared to the free form, even enhanced or a bit reduced compared to the level of activity of the free form of the drug (a bit reduced as used herein refers to a reduction of not more than 20% of the activity of the free form of a drug measured under the same conditions as the activity of a drug). The skilled person understands that the activity of a drug can be measured by standard procedures known in the art which of course depend on the activity of such a drug.

The term “functionalized” drug or polymer refers to a polymer or drug (such as a registered drug), wherein a reactive group selected from the group consisting of —OH, —SH, —NH₂, —N((C₂-C₅)alkyl)H, L₁-N(H)—C(O)-L₂, wherein the amide function is part of a lactam of Re and L₁ and L₂ represent carbons of the lactam cycle, —C(O)OH, —C(O)NH₂, —S(O₂)OH, and a water soluble salt or anhydride of any of the foregoing was replaced by a residue comprising a —N₃, ethynyl, ethenyl, a conjugated diene, a isonitrile, or a tetrazine moiety. Such a functionalization can be achieved by methods well known in the art, e.g., by, for example, solid-phase peptide synthesis methods (Palomo et al. RSC Adv. 2014, 4, 32658-32672) and post synthesis modification (Bernardes et al. Chem. Rev. 2015, 115, 2174-2195) in the case of peptides and antibodies. In the case of non-peptides drugs, the skilled person is aware of the reactivity of the functional groups that exists in the drug that could be used for introducing a clickable moiety. The term “lactame” as used herein refers to a cyclic structure comprising at least one amide function as part of the cycle. Encompassed are those lactames, wherein a nitrogen atom of such an amide function has a hydrogen attached to it such as in 2,5-diketopiperazin, a 2,5-diketopiperazinyl residue or a derivative of any of the two foregoing.

The term “modified” in connection with any residue Re and/or Rf (drug and/or polymer as used herein) refers to a modification wherein a reactive group of a free drug or polymer molecule is “replaced” (substituted) by the bond to W or Y in any of the conjugates according to the invention (see, e.g., formula (B), (B′), (C) and (C′), respectively). Non-limiting examples of reactive groups of the free form of a drug or polymer are —OH, —SH, —NH₂, —N((C₂-C₅)alkyl)H, (L₁)—NH—C(O)-(L₂) wherein L₁ and L₂ are carbons of the lactam cycle, —C(O)OH, —C(O)NH₂, —S(O₂)OH, and a water soluble salt or anhydride of any of the foregoing), —N₃ ethynyl a conjugated alkdiene, ethenyl, isonitrile (isocyanide), tetrazine. In the conjugates according to the invention such a reactive group is replaced by a correlating W or Y, respectively, e.g. an —OH group of a free form of a polymer or drug is replaced by *—O— or *—O—C(O)— in a conjugate according to the invention, wherein * indicates the bond to V and X in a conjugate of the invention as described herein.

For example, naproxen, a pain killer, is 2-(6-methoxy-2-naphthyl)propionic acid (which is also the free form of naproxen). The replacement of the reactive —COOH group of naproxen by a linker/Re (or linker/Rf) residue is demonstrated in Example 2a (and its release in, e.g., FIG. 1). The skilled person will understand that naproxen does not contain any further reactive group which could be replaced so no further modification as defined herein is possible for this molecule. On the other hand, e.g., cellulose is known as a molecule with many —OH groups. Thus, not only one —OH group can be replaced, by a linker/Rf residue. Furthermore, depending on the structure of a polymer or drug, such as a peptide, the skilled person understands that a polymer or drug may comprise different types of reactive groups (there can be one or more than one reactive group of each type). For example, a polymer or drug may comprise one or more hydroxy group(s) and one or more carboxyl group(s) as reactive groups. The skilled person understands that it is irrespective at which position a drug or polymer is attached to a linker molecule forming a conjugate according to the invention. The goal of the conjugates of the invention is to immobilize a drug as known in the art and, upon radiation with UV-light, release said drug as known in the art (e.g. naproxen) from said conjugate (see, e.g., FIG. 1).

The term “non-toxic” as used herein refers to a conjugate according to the invention and/or by-products of the release of the free form of a drug or a free form derivative according to the invention which has at least one of the three characteristics selected from the group consisting of biocompatible, biodegradable and bioresorbable.

The term “reactive group” as used herein refers to any group (except of a diazine group) which can react with a compatible reactive group. Non-limiting examples of reactive groups are hydroxy (—OH), thiol (—SH), amin (—NH₂), halides such as Cl, Br, F or I, a cyclic amide function (—N(H)—C(O)—) such as in a lactame, wherein the free bond of the nitrogen and the free bond of the C(O) carbon each represents a bond to a neighbored carbon atom of the cycle, sulfonic acid (—S(O₂)OH), —O—S(O₂)OH, thiocarboxylic acid (—C(S)OH), carboxyl (carboxylic acid (—C(O)OH)), a water soluble salt, such as a pharmaceutically acceptable salt, thereof, a carboxylic acid halide and anhydrates thereof), amide (—C(O)NH₂), carboxylic acid ester, preferably with (C₁-C₅)alkyl, isothiocyanate (—NCS), isocyanate (—NCO) such as —(C₁-C₅)—NCO, —NCO, —O—(C₁-C₅)—NCO, —O—NCO, —O—C(O)—(C₁-C₅)—NCO, —O—C(O)—NCO, —C(O)O—NCO or —C(O)O—(C₁-C₅)NCO, hydrazine, a water soluble salt, such as a pharmaceutically acceptable salt, of any of the foregoing, alkyne such as -ethynyl, —O—(C₁-C₅)alkyl-ethynyl, —C(O)O—(C₁-C₅)alkyl-ethynyl or —O—C(O)—(C₁-C₅)alkyl-ethynyl, azide, such as —N₃, —(C₁-C₅)alkyl-N₃, —O—(C₁-C₅)alkyl-N₃, —O—C(O)—(C₁-C₅)alkyl-N₃ or —C(O)O—(C₁-C₅)alkyl-N₃, maleimide, imidate, alkenyl such as —(C₃—C)-alkenyl, —O—(C₃—C)-alkenyl, —C(O)—O—(C₃-C₇)-alkenyl, —O—C(O)—(C₃—C)-alkenyl, a conjugated diene such as a conjugated —(C₄-C₁₀)alkdienyl, a conjugated —O—(C₅-C₁₀)alkdienyl, a conjugated —O—C(O)—(C₅-C₁₀)alkdienyl or —C(O)O—(C₅C₁₀)alkdienyl, tetrazinyl such as —(C₁-C₅)alkyl-tetrazinyl, -tetrazinyl, —O—(C₁-C₅)tetrazinyl, —O-tetrazinyl, —O—C(O)—(C₁-C₅)tetrazinyl, —O—C(O)-tetrazinyl, —C(O)O-tetrazinyl or —C(O)O—(C₁-C₅)tetrazinyl.

The term “substitution” of a reactive group or a “substituted” reactive group as used herein means that a specific reactive group of a drug or polymer which is present in an unmodified state (free form) of a polymer or drug known in the art reacts with a corresponding reactive group of a linker molecule to form a conjugate and is thereby replaced by group W or Y, respectively, as identified in the conjugates of formula (A1). The skilled person understands that part of a “substituted” reactive group (such as a heteroatom) can still be part of the new formed conjugate (e.g. the O-atom of a hydroxy group of a polymer will be part of an ester group when the hydroxyl group reacts with a carboxyl group of a linker molecule and, thus, the hydroxy group is replaced by said ester function), but the reactive group as such does not exist as in the free form of a polymer or drug due to the covalent bond to the linker molecule or the linker of a linker/drug conjugate. The skilled person will also understand that the free form of a drug in view of this covalent bond to the linker is restored, when the linker breaks down by applying UV-light and therefore releasing the drug which reactive group at this position is thereby reinstalled or, in case of a click chemistry reaction, a free form derivative is restored.

It is understood that, as long as a combination does not violate any law of nature, all embodiments of the present invention, irrespective of the fact if an embodiment is an embodiment, a preferred embodiment, a more preferred embodiment, a particularly preferred embodiment or a most preferred embodiment, can be combined and such combinations of two or more embodiments of the invention are disclosed herein by disclosing such embodiments.

### FIGURES

FIG. 1 shows the Naproxen release from a conjugate according to the invention upon UV irradiation.

FIG. 2 shows the UV-Vis spectrum of two linker molecules according to the invention.

FIG. 3 shows the concept of a depot according to the invention with an exemplary remote control device as control device.

FIG. 4 shows the spectrum of a suitable UV-light source with a significant amount of photons between 320 nm and 380 nm.

FIG. 5 shows the release profile of 7-hydroxy coumarin.

FIG. 6: shows the concentration of the released 7-hydroxy coumarin.

FIG. 7 shows the amount of 7-hydroxy coumarin determined by emission spectroscopy (Abs₍ₘₐₓ₎=325 nm. Em₍ₘₐₓ₎=450 nm) correlated to the pulse of irradiation using a 366 nm comprising light source.

FIG. 8 shows the synthesis of conjugate B with GLP-1 alkyne.

FIG. 9 shows the photo release of GLP-1 derivative (free form derivative).

FIG. 10 shows the photo release GLP-1 derivative scheme and structure of GLP-1-derivative.

FIG. 11 shows the photo release of a free form derivative of glucagon.

FIG. 12 shows the photo release of a free form derivative of insulin.

### DETAILED DESCRIPTION

### Drug Conjugates

One aspect of the present invention refers to a drug conjugate of formula (A1)

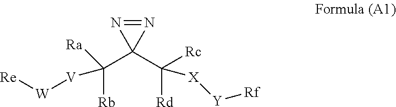

wherein
Ra and Rb independently from each other represents a moiety selected from H, halogen (preferably F, Cl, I or Br), (C₁-C₅)alkyl, (C₂-C₅)alkenyl, a five- or six-membered heterocycle, (C₃₋₃₀)cycloalkyl (preferably cyclopropyl, cyclopentyl, cyclohexanyl, adamantyl (C₁₀H₁₅), iceanyl (C₁₂H₁₇), diadamantanyl (C₁₄H₁₉), triadamantyl (C₂₂H₂₃), isotetramantanyl (C₂₂H₂₇), pentamantanyl (C₂₆H₃₁), cyclohexamantanyl (C₂₆H₂₉), superadamantan (C₃₀H₃₅)), phenyl, benzyl (—CH₂—C₆H₅) each heterocycle, cycloalkyl, phenyl or benzyl can optionally be substituted with 1, 2, 3, 4 or 5 substituents selected from the group consisting of hydroxy, halogen, (C₁-C₅)alkyl, (C₁-C₅)alkoxy; or
Ra and Rb form together with the carbon they are attached to a five- or six-membered heterocycle or (C₃₋₁₂)cycloalkyl, preferably cyclopentyl or cyclohexyl optionally substituted with 1, 2, 3, 4 or 5 substituents selected from the group consisting of hydroxy, halogen, (C₁-C₅)alkyl, (C₁-C₅)alkoxy); or
Ra and Rb represent together with the carbon they are attached to and V a (C₃₋₃₀)cycloalkyl moiety to which —W—Re is attached wherein the cycloalkyl moiety is selected from the group consisting of adamantyl (C₁₀H₁₄), iceanyl (C₁₂H₁₆), diadamantanyl (C₁₄H₁₈), triadamantyl (C₂₂H₂₂), isotetramantanyl (C₂₂H₂₆), pentamantanyl (C₂₆H₃₀), cyclohexamantanyl (C₂₆H₂₈), superadamantan (C₃₀H₃₄), each optionally substituted with 1, 2, 3, 4 or 5 substituents selected from the group consisting of hydroxy, halogen, (C₁-C₅)alkyl, (C₁-C₅)alkoxy; more preferably the cycloalkyl is selected from the group consisting of adamantyl (C₁₀H₁₄), iceanyl (C₁₂H₁₆), diadamantanyl (C₁₄H₁₈), triadamantyl (C₂₂H₂₂), isotetramantanyl (C₂₂H₂₆), pentamantanyl (C₂₆H₃₀), cyclohexamantanyl (C₂₆H₂₈), superadamantan (C₃₀H₃₄), more preferably the cycloalkyl is selected from the group consisting of adamantyl (C₁₀H₁₄), iceanyl (C₁₂H₁₆), diadamantanyl (C₁₄H₁₈), even more preferably adamantyl (C₁₀H₁₄)); or
Ra and Rb form together with the carbon they are attached to and V a phenyl moiety to which —W—Re is attached;
Rc and Rd independently from each other represents a moiety selected from H, hydroxy, halogen (preferably F, Cl, I or Br), (C₁-C₅)alkyl, (C₂-C₅)alkenyl (preferably Rc and Rd each represent H);
V and X independently from each other represents a moiety selected from the group consisting of a bond (i.e. in such a case W and Y, respectively, are directly bound to the carbon to which Ra/Rb and Rc/Rd, respectively, are attached), or a (C₁-C₅)alkyl, preferably selected from the group consisting of —CH₂—, —CH₂—CH₂—, —CH₂—CH₂—CH₂—, —CH₂—CH₂—CH₂—CH₂—, or —CH₂—CH₂—CH₂—CH₂—CH₂— (most preferably X represents a bond); or
V forms together with Ra and Rb and with the carbon they are attached to a cycloalkyl selected from the group consisting of adamantyl (C₁₀H₁₅), iceanyl (C₁₂H₁₇), diadamantanyl (C₁₄H₁₉), triadamantyl (C₂₂H₂₃), isotetramantanyl (C₂₂H₂₇), pentamantanyl (C₂₆H₃₁), cyclohexamantanyl (C₂₆H₂₉), superadamantan (C₃₀H₃₅); or
V forms together with Ra, Rb and with the carbon they are attached to a phenyl moiety which is substituted with —W—Re;
W and Y independently from each other represents a moiety selected from the group consisting of *—O—, *—S—, *—NH—, *—N((C₁-C₅)alkyl)-, *-((L₁)-N)—C(O)-(L₂) wherein L₁ and L₂ are carbons of a lactame cycle of Rₑ or R_{f}, respectively, *—C(O)—O—, *—O—C(O)—, *—O—C(O)—NH—, *—S(O₂)—O—, *—O—S(O₂)—, *—C(S)—O—, *—O—C(S)—, *—C(O)—NH—, *—NH—C(O)—, *—S(O₂)—NH—, *—NH—S(O₂)—, *-triazol-, *—O—(C₂-C₅)alkyl-triazol-, *—O—C(O)—(C₂-C₅)alkyl-triazol-, *—C(O)O—(C₂-C₅)alkyl-triazol-, *-cyclohexenyl-, *—O-cyclohexenyl- *—O—(C₁-C₅)alkyl-cyclohexenyl-, *—O—C(O)—(C₁-C₅)alkyl-cyclohexenyl-, *—O—C(O)-cyclohexenyl-*—C(O)O—(C₁-C₅)alkyl-cyclohexenyl-, *—C(O)O-cyclohexenyl- wherein each cyclohexenyl moiety is optionally further substituted with 1, 2 or 3 (C₁-C₄)alkyl,

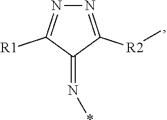

wherein R1 represents H or (C₁-C₅)alkyl and R₂ represents —(C₁-C₅)-alkyl-O— (bond to Re or Rf, respectively), —(C₁-C₅)-alkyl-O—C(O)— (bond to Re or Rf, respectively), —(C₁-C₅)-alkyl-C(O)O— (bond to Re or Rf, respectively),
wherein * indicates the bond to V and X, respectively, and wherein the other bond is a bond to a carbon (or in case of a lactam to two carbons) of Re or Rf, respectively;
Re is either a modified polymer (preferably selected from the group consisting of polyacrylates (PA) (e.g., poly(hydroxyethylmethacrylate)), polymethacrylates (e.g. poly(hydroxyethylmethacrylate) (PHEMA)), polyacrylamides (e.g. poly(N-(2-hydroxypropyl)acrylamide)), polymethacrylamides (e.g. poly[N-(2-hydroxypropyl)]methacrylamide) (HPMA)), poly(alpha-hydroxy acids), poly(lactide-co-glycolides) (PLGA), polylactides (PLA), polyglycolides (PG), functionalized polystyrenes, polyethylene glycol (PEG), poly(alpha-hydroxy acids), polyorthoesters (POE), N-vinyl pyrrolidone, polyaspirins, polyphosphagenes, dendrimers, polyamides, proteins or peptides (e.g. albumin, collagen or fibrin), polysaccharides (e.g. cyclodextrine, agarose, starch, hyaluronic acid, chitosan, gelatin, or alginates) (in one more preferred embodiment selected from the group consisting of polyacrylates, polymethacrylamides, functionalized polystyrenes and polyamides;
such as PHEMA or HPMA; in another preferred embodiment selected from the group consisting of poly(alpha-hydroxy acids), PLGA, PLA, PG, PEG, poly(alpha-hydroxy acids), POE, dendrimers, polyaspirins, polyphosphagenes, dendrimers, proteins (e.g. albumin), peptides, polysaccharides; such as poly(alpha-hydroxy acids), PLGA, PLA, PG, PEG, poly(alpha-hydroxy acids), POE, albumin, collagen or fibrin, cyclodextrine, agarose, starch, hyaluronic acid, chitosan, gelatin, or alginates.
or
Re is a modified drug (preferably selected from the group consisting of ACE-inhibitors; anti-anginal drugs; anti-arrhythmias; anti-asthmatics; anti-cholesterolemics; anti-convulsants; anti-depressants; anti-diarrhea preparations; anti-histamines; anti-hypertensive drugs; anti-infectives; anti-inflammatory agents; anti-lipid agents; anti-manics; anti-nauseants; anti-stroke agents; anti-thyroid preparations; anti-tumor drugs; anti-tussives; anti-uricemic drugs; anti-viral agents; acne drugs; alkaloids; amino acid preparations; anabolic drugs; analgesics; anesthetics; angiogenesis inhibitors; antacids; anti-arthritics; antibiotics; anticoagulants; antiemetics; antiobesity drugs; antiparasitics; antipsychotics; antipyretics; antispasmodics; antithrombotic drugs; anxiolytic agents; appetite stimulants; appetite suppressants; beta blocking agents; bronchodilators; cardiovascular agents; cerebral dilators; chelating agents; cholecystokinin antagonists; chemotherapeutic agents; cognition activators; contraceptives; coronary dilators; cough suppressants; decongestants; deodorants; dermatological agents; diabetes agents; diuretics; emollients; enzymes; erythropoietic drugs; expectorants; fertility agents; fungicides; gastrointestinal agents; growth regulators; hormone replacement agents; hyperglycemic agents; hypnotics; hypoglycemic agents; laxatives; migraine treatments; mineral supplements; mucolytics; narcotics; neuroleptics; neuromuscular drugs; NSAIDS; nutritional additives; peripheral vasodilators; polypeptides; prostaglandins; psychotropics; renin inhibitors; respiratory stimulants; steroids; stimulants; sympatholytics; thyroid preparations; tranquilizers; uterine relaxants; vaginal preparations; vasoconstrictors; vasodilators; vertigo agents; vitamins; wound healing agents; and contrast agents); wherein the modification in the modified Re is the replacement of a reactive group of the free form of Re (preferably wherein said reactive group is selected from the group consisting of —OH, —SH, —NH₂, —N((C₂-C₅)alkyl)H, L₁-N(H)—C(O)-L₂, wherein the amide function is part of a lactam of Re and L₁ and L₂ represent carbons of the lactam cycle, —C(O)OH, —C(O)NH₂, —S(O₂)OH, and a water soluble salt or anhydride of any of the foregoing,
by W (W represents a moiety which can be formed by reacting a compatible group with one of the preferred reactive groups described above, e.g. W may be *—O— or *—C(O)—O— when the reactive group of the free form of Re was —OH); or
wherein the modification is the replacement of a reactive group of a free form of a functionalized Re selected from the group consisting of —N₃, ethynyl, ethenyl, a conjugated diene, a tetrazine moiety and isonitrile, preferably a reactive group is selected from the group consisting of —N₃ and -ethynyl,
by W and wherein W represents in such a case a moiety selected from the group consisting of *-triazol-, *—O—(C₂-C₅)alkyl-triazol-, *—O—C(O)—(C₂-C₅)alkyl-triazol-, *—C(O)O—(C₂-C₅)alkyl-triazol-, *-cyclohexenyl-, *—O—(C₂-C₅)alkyl-cyclohexenyl-, *—O—C(O)—(C₂-C₅)alkyl-cyclohexenyl-,*—C(O)O—(C₂-C₅)alkyl-cyclohexenyl-, and *—S—CH₂—C₂—,

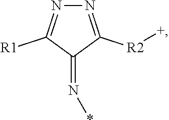

wherein R1 represents H or (C₁-C₅)alkyl and R₂ represents —(C₁-C₅)-alkyl-O— (bond to Re), —(C₁-C₅)-alkyl-O—C(O)— (bond to Re), —(C₁-C₅)-alkyl-C(O)O— (bond to Re), preferably the moiety is selected from the group consisting of *-triazol-, *—O—(C₂-C₅)alkyl-triazol-, *—O—C(O)—(C₂-C₅)alkyl-triazol-, *—C(O)O—(C₂-C₅)alkyl-triazol-, wherein * indicates the bond to V, and wherein the other bond is a bond to a carbon of Re;
Rf represents a modified drug (preferably selected from the group consisting of ACE-inhibitors; anti-anginal drugs; anti-arrhythmias; anti-asthmatics; anti-cholesterolemics; anti-convulsants; anti-depressants; anti-diarrhea preparations; anti-histamines; anti-hypertensive drugs; anti-infectives; anti-inflammatory agents; anti-lipid agents; anti-manics; anti-nauseants; anti-stroke agents; anti-thyroid preparations; anti-tumor drugs; anti-tussives; anti-uricemic drugs; anti-viral agents; acne drugs; alkaloids; amino acid preparations; anabolic drugs; analgesics; anesthetics; angiogenesis inhibitors; antacids; anti-arthritics; antibiotics; anticoagulants; antiemetics; antiobesity drugs; antiparasitics; antipsychotics; antipyretics; antispasmodics; antithrombotic drugs; anxiolytic agents; appetite stimulants; appetite suppressants; beta blocking agents; bronchodilators; cardiovascular agents; cerebral dilators; chelating agents; cholecystokinin antagonists; chemotherapeutic agents; cognition activators; contraceptives; coronary dilators; cough suppressants; decongestants; deodorants; dermatological agents; diabetes agents; diuretics; emollients; enzymes; erythropoietic drugs; expectorants; fertility agents; fungicides; gastrointestinal agents; growth regulators; hormone replacement agents; hyperglycemic agents; hypnotics; hypoglycemic agents; laxatives; migraine treatments; mineral supplements; mucolytics; narcotics; neuroleptics; neuromuscular drugs; NSAIDS; nutritional additives; peripheral vasodilators; polypeptides; prostaglandins; psychotropics; renin inhibitors; respiratory stimulants; steroids; stimulants; sympatholytics; thyroid preparations; tranquilizers; uterine relaxants; vaginal preparations; vasoconstrictors; vasodilators; vertigo agents; vitamins; wound healing agents; and contrast agents;
wherein the modification in the modified Rf is the replacement of a reactive group of the free form of Rf (preferably wherein said reactive group is selected from the group consisting of —OH, —SH, —NH₂, —N((C₂-C₅)alkyl)H, L₁-N(H)—C(O)-L₂, wherein the amide function is part of a lactam of Rf and L₁ and L₂ represent carbons of the lactam cycle, —C(O)OH, —C(O)NH₂, —S(O₂)OH, and a water soluble salt or anhydride of any of the foregoing,
by Y (Y represents a moiety which can be formed by reacting a compatible group with one of the preferred reactive groups described above, e.g. Y may be *—O— or *—C(O)—O— when the reactive group of the free form of Re was —OH); or
wherein the modification is the replacement of a reactive group of the free form of a functionalized Rf selected from the group consisting of —N₃, ethynyl, ethenyl, a conjugated diene, a tetrazine moiety and isonitrile, preferably a reactive group is selected from the group consisting of —N₃ and -ethynyl,
by Y and wherein Y represents in such a case a moiety selected from the group consisting of *-triazol-, *—O—(C₂-C₅)alkyl-triazol-, *—O—C(O)—(C₂-C₅)alkyl-triazol-, *—C(O)O—(C₂-C₅)alkyl-triazol-, *-cyclohexenyl-, *—O—(C₂-C₅)alkyl-cyclohexenyl-, *—O—C(O)—(C₂-C₅)alkyl-cyclohexenyl-,*—C(O)O—(C₂-C₅)alkyl-cyclohexenyl-, and *—S—CH₂—C₂—,

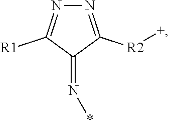

wherein R1 represents H or (C₁-C₅)alkyl and R₂ represents —(C₁-C₅)-alkyl-O— (bond to Rf), —(C₁-C₅)-alkyl-O—C(O)— (bond to Rf), —(C₁-C₅)-alkyl-C(O)O— (bond to Rf), preferably the moiety is selected from the group consisting of *-triazol-, *—O—(C₂-C₅)alkyl-triazol-, *—O—C(O)—(C₂-C₅)alkyl-triazol-, *—C(O)O—(C₂-C₅)alkyl-triazol-,
wherein * indicates the bond to X, and wherein the other bond is a bond to a carbon of Rf;
optionally, Re may be further modified by at least one further substitution of a further reactive group of such a modified drug or polymer (preferably, wherein said reactive group is selected from the group consisting of —OH, —SH, —NH₂, —N((C₂-C₅)alkyl)H, L₁-N(H)—C(O)-L₂, wherein the amide function is part of a lactam of Re and L₁ and L₂ represent carbons of the lactam cycle, —C(O)OH, —C(O)NH₂, —S(O₂)OH, and a water soluble salt or anhydride of any of the foregoing) an azide-, an alkyne-, a conjugated alkdiene-, an alkene-, an isonitrile- (isocyanide-), a tetrazine-moiety, more preferably —OH, —SH, —NH₂, —C(O)OH, —C(O)NH₂, —N(H)—C(O)— of a lactame, —S(O₂)OH, and a water soluble salt of any of the foregoing), by a linker/Rf residue of formula (B)

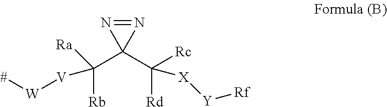

wherein # represents the bond to the further modified Re; and/or
optionally Rf may be further modified by at least one further substitution of a further reactive group of such a modified drug, (preferably, wherein said reactive group is selected from the group consisting of —OH, —SH, —NH₂, —N((C₂-C₅)alkyl)H, L₁-N(H)—C(O)-L₂, wherein the amide function is part of a lactam of Rf and L₁ and L₂ represent carbons of the lactam cycle, —C(O)OH, —C(O)NH₂, —S(O₂)OH, and a water soluble salt or anhydride of any of the foregoing) an azide-, an alkyne-, a conjugated alkdiene-, an alkene-, an isonitrile- (isocyanide-), a tetrazine-moiety, by a linker/Re residue of formula (C)

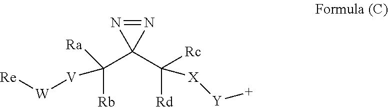

wherein + represents the bond to the further modified Rf; or
a drug conjugate of formula (A1′)

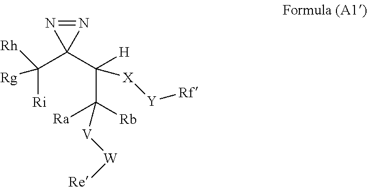

wherein V, W, Y, Ra and Rb are as defined for formula (A1) and any of its preferred embodiments;
Rh, Rg and Ri each individually represent halogen, preferably F, I or Cl, (C₁-C₅)alkyl, phenyl, benzyl; or
Rh, Rg and Ri form together with the carbon to which they are attached an adamantly moiety;
Re′ and Rf′ are identical to Re and Rf as defined for formula (A1) with the exception that an optionally further modified Re′ refers to a modification of Re′ in a conjugate of formula (A1′) with at least one further linker/Rf′ residue of formula (B′) and an optionally further modified Rf′ refers to a modification of Rf′ in a conjugate of formula (A1′) with at least one further linker/Re′ residue of formula (C′):
optionally, Re′ in Formula (A1′) may be further modified by at least one further substitution of a further reactive group of such a modified drug (preferably, wherein said reactive group is selected from the group consisting of —OH, —SH, —NH₂, —N((C₂-C₅)alkyl)H, L₁-N(H)—C(O)-L₂, wherein the amide function is part of a lactam of Re′ and L₁ and L₂ represent carbons of the lactam cycle, —C(O)OH, —C(O)NH₂, —S(O₂)OH, and a water soluble salt or anhydride of any of the foregoing) an azide-, an alkyne-, a conjugated alkdiene-, an alkene-, an isonitrile- (isocyanide-), a tetrazine-moiety, by a linker/Rf′ residue of formula (B′)

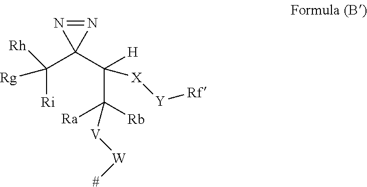

wherein # represents the bond to the further modified Re′; and/or
optionally Rf′ may be further modified by at least one further substitution of a further reactive group of such a modified drug, (preferably, wherein said reactive group is selected from the group consisting of —OH, —SH, —NH₂, —N((C₂-C₅)alkyl)H, L₁-N(H)—C(O)-L₂, wherein the amide function is part of a lactam of Rf′ and L₁ and L₂ represent carbons of the lactam cycle, —C(O)OH, —C(O)NH₂, —S(O₂)OH, and a water soluble salt or anhydride of any of the foregoing) an azide as described herein, an azide-, an alkyne-, a conjugated alkdiene-, an alkene-, an isonitrile- (isocyanide-), a tetrazine-moiety, by a linker/Re′ residue of formula (C′)

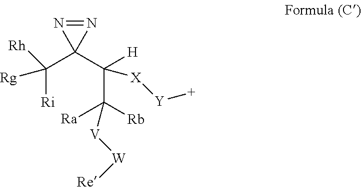

wherein + represents the bond to the further modified Rf′.

The skilled person understands that a C-atom of Re (or Rf or Re′ or Rf′, respectively; or two carbon atoms in the case of a amide group of a lactam) which is attached to W (or Y, respectively) is a C-atom to which in the free form of the drug or polymer, respectively, a functional group is attached which now forms in a drug conjugate of the invention together with a respective functional group Y′ or W′ of a linker molecule of formula D1 any of the two space holder Y or W. For example, a linker molecule (D1) having a —C(O)OH or a salt of —C(O)OH at position Y′ may react with a hydroxy group of insulin forming the —C(O)O-insulin=—Y—Rf residue or —W—Re residue in formula (A1). When the conjugate breaks down by UV-light, insulin is set free with a restored hydroxy-group at said former position.

The skilled person understands that preferably further drug molecules will be attached at other positions of a drug or polymer to said drug or polymer. For example, agarose (a polymer) has more than one reactive —OH groups to which a linker group can be attached, thereby modifying the agarose according to the present invention. Thus, if a conjugate according to the present invention comprises agarose as a polymer and insulin as a dug which are connected via the linker in formula (A1) (or (A1′)), further insulin molecules can be attached to said agarose and/or further insulin molecules or agarose molecules can be attached via a linker according to the invention to the conjugate according to the invention. The skilled person understands how to choose the same or different reactive group pairs to synthesize conjugates according to the present invention with reasonable yield and purity.

In one preferred embodiment, at least one substituent selected from the group consisting of Ra, Rb, Rc and Rd represents H.

In yet another preferred embodiment, Rc or Rd in formula (A1) represent H.

In yet another preferred embodiment, Rc and Rd in formula (A1) each represent H.

In one preferred embodiment, X in formula (A1) or (A1′) represents a bond.

In yet another preferred embodiment, Rc and Rd in formula (A1) each represent H and X represents a bond.

In yet another preferred embodiment, at least one of Rc and Rd in formula (A1) represent H and X represents a bond and Ra and Rb represent a substituent which is not H.

In yet another preferred embodiment, Y in formula (A1) or (A1′) represents a moiety selected from the group consisting of *—O—, *—S—, *—NH—, *—N((C₁-C₅)alkyl)-, *-((L₁)-N)—C(O)-(L₂) wherein L₁ and L₂ are carbons of a lactam cycle of Rf, *—O—C(O)—, *—O—C(S)—*—O—C(O)—NH—, *—S(O₂)—O—, *—O—S(O₂)—, *—O—C(S)—, *—NH—C(O)—, *—S(O₂)—NH—, *—NH—S(O₂)—, *-triazol-, *—O—(C₂-C₅)alkyl-triazol-, *—O—C(O)—(C₂-C₅)alkyl-triazol-, *—C(O)O—(C₂-C₅)alkyl-triazol-.

In yet another preferred embodiment, Y in formula (A1) or (A1′) represents a moiety selected from the group consisting of *—O—, *—S—, *—NH—, *—N((C₁-C₅)alkyl)-, *-((L₁)-N)—C(O)-(L₂) wherein L₁ and L₂ are carbons of a lactam cycle of Rf (representing, thus, in such a case not one but two bonds to Rf), *—O—C(O)—, *—O—C(S)—, *—O—C(O)—NH—, *—S(O₂)—O—, *—O—S(O₂)—, *—O—C(S)—, *—NH—C(O)—, *—S(O₂)—NH—, *—NH—S(O₂)—.

In a yet more preferred embodiment, Y in formula (A1) or (A1′) represents a moiety selected from the group consisting of *—O—, *—O—C(O)—; *—O—C(O)—NH—, *—S—, *—NH—C(O)—, *—N((C₁-C₅)alkyl)-, *-((L₁)-N)—C(O)-(L₂) wherein L₁ and L₂ are carbons of a lactam cycle of Rf.

In one preferred embodiment, W in formula (A1) or (A1′) represents a moiety selected from the group consisting of *—O—, *—S—, *—NH—, *—N((C₁-C₅)alkyl)-, *—O—C(O)—, *—O—C(O)—NH—, *—S(O₂)—O—, *—O—S(O₂)—, *—NH—C(O)—, *—S(O₂)—NH—, *—NH—S(O₂)—, *-triazol-, *—O—(C₂-C₅)alkyl-triazol-, *—O—C(O)—(C₂-C₅)alkyl-triazol-, *—C(O)O—(C₂-C₅)alkyl-triazol.

In a more preferred embodiment, W represents a moiety selected from the group consisting of *—O—, *—S—, *—NH—, *—N((C₁-C₅)alkyl)-, *—O—C(O)—, *—O—C(O)—NH—, *—NH—C(O)—, *-triazol-, *—O—(C₂-C₅)alkyl-triazol-, *—O—C(O)—(C₂-C₅)alkyl-triazol-, *—C(O)O—(C₂-C₅)alkyl-triazol-.

In one preferred embodiment, W represents a moiety selected from the group consisting of *—O—, *—O—C(O)—, *—N((C₁-C₅)alkyl)-, *-((L₁)-N)—C(O)-(L₂) wherein L₁ and L₂ are carbons of a lactam cycle of Rf (representing, thus, in such a case not one but two bonds to Rf) and *—NH—. *-triazol-, *—O—(C₂-C₅)alkyl-triazol-, *—O—C(O)—(C₂-C₅)alkyl-triazol-, *—C(O)O—(C₂-C₅)alkyl-triazol-, and Y represents a moiety selected from the group consisting of *—O—, *—O—C(O)—, *—N((C₁-C₅)alkyl)-, *-((L₁)-N)—C(O)-(L₂) wherein L₁ and L₂ are carbons of a lactam cycle of Rf (representing, thus, in such a case not one but two bonds to Rf) and *—NH—. *-triazol-, *—O—(C₂-C₅)alkyl-triazol-, *—O—C(O)—(C₂-C₅)alkyl-triazol-, *—C(O)O—(C₂-C₅)alkyl-triazol-.

In yet another preferred embodiment, W represents a moiety selected from the group consisting of *—O—, *—O—C(O)—, *—N((C₁-C₅)alkyl)-, *-((L₁)-N)—C(O)-(L₂) wherein L₁ and L₂ are carbons of a lactam cycle of Rf (representing, thus, in such a case not one but two bonds to Rf) and *—NH—, *-triazol-, *—O—(C₂-C₅)alkyl-triazol-, *—O—C(O)—(C₂-C₅)alkyl-triazol-, *—C(O)O—(C₂-C₅)alkyl-triazol-, and Y represents a moiety selected from the group consisting of *—O—, *—O—C(O)—, *—N((C₁-C₅)alkyl)-, *-((L₁)-N)—C(O)-(L₂) wherein L₁ and L₂ are carbons of a lactam cycle of Rf (representing, thus, in such a case not one but two bonds to Rf) and *—NH—, *—S—. *-triazol-, *—O—(C₂-C₅)alkyl-triazol-, *—O—C(O)—(C₂-C₅)alkyl-triazol-, *—C(O)O—(C₂-C₅)alkyl-triazol-; and Y and W are different from each other (i.e. W and Y do not represent the same type of moiety).

In yet another preferred embodiment, W represents a moiety selected from the group consisting of *—O—, *—O—C(O)—, *—N((C₁-C₅)alkyl)-, *-((L₁)-N)—C(O)-(L₂) wherein L₁ and L₂ are carbons of a lactam cycle of Rf (representing, thus, in such a case not one but two bonds to Rf) and *—NH—. *-triazol-, *—O—(C₂-C₅)alkyl-triazol-, *—O—C(O)—(C₂-C₅)alkyl-triazol-, *—C(O)O—(C₂-C₅)alkyl-triazol-, and Y represents a moiety selected from the group consisting of *—O—, *—O—C(O)—, *—N((C₁-C₅)alkyl)-, *-((L₁)-N)—C(O)-(L₂) wherein L₁ and L₂ are carbons of a lactam cycle of Rf (representing, thus, in such a case not one but two bonds to Rf), *—NH—, and *—S—.

In yet another preferred embodiment, W represents a moiety selected from the group consisting of *—O—, *—O—C(O)—, *—N((C₁-C₅)alkyl)-, *-((L₁)-N)—C(O)-(L₂) wherein L₁ and L₂ are carbons of a lactam cycle of Rf (representing, thus, in such a case not one but two bonds to Rf) and *—NH—. *-triazol-, *—O—(C₂-C₅)alkyl-triazol-, *—O—C(O)—(C₂-C₅)alkyl-triazol-, *—C(O)O—(C₂-C₅)alkyl-triazol-, and Y represents a moiety selected from the group consisting of *—O—, *—O—C(O)—, *—N((C₁-C₅)alkyl)-, *-((L₁)-N)—C(O)-(L₂) wherein L₁ and L₂ are carbons of a lactam cycle of Rf (representing, thus, in such a case not one but two bonds to Rf), *—NH—, and *—S—; and Y and W are different from each other.

In yet another preferred embodiment, W represents a moiety selected from the group consisting of *—O—, *—O—C(O)—, *—N((C₁-C₅)alkyl)-, *-((L₁)-N)—C(O)-(L₂) wherein L₁ and L₂ are carbons of a lactam cycle of Rf (representing, thus, in such a case not one but two bonds to Rf) and *—NH—. *-triazol-, *—O—(C₂-C₅)alkyl-triazol-, *—O—C(O)—(C₂-C₅)alkyl-triazol-, *—C(O)O—(C₂-C₅)alkyl-triazol-, Rf is a peptide and Y represents a moiety selected from the group consisting of *-triazol-, *—O—(C₂-C₅)alkyl-triazol-, *—O—C(O)—(C₂-C₅)alkyl-triazol-, *—C(O)O—(C₂-C₅)alkyl-triazol-, *—O—, *—O—C(O)—, *—N((C₁-C₅)alkyl)-, *-((L₁)-N)—C(O)-(L₂) wherein L₁ and L₂ are carbons of a lactam cycle of Rf (representing, thus, in such a case not one but two bonds to Rf), *—NH—, and *—S—.

In another preferred embodiment, Y and W in formula (A1) or (A1′) are identical. The skilled person is aware how to protect a reactive group out of two reactive groups of the same type in an asymmetric molecule or how to calculate reactive partners to only react 50% of two reactive groups of the same type in a symmetric or, preferably, asymmetric molecule.

However, in a more preferred embodiment, Y and W in formula (A1) or (A1′) are not identical. Y and W, and, thus, Y′ and W′ of a linker molecule (see below) are different (reactive) groups: By choosing two different reactive groups in a linker molecule, the resulting drug conjugate of formula (A1) or (A1′) comprises two different groups W and Y, respectively. Such a choice can enhance the stereoselective reaction of such linker molecules to form a drug conjugate of formula (A1) or (A1′), respectively.

In an even more preferred embodiment, Y represents *—O—, *—O—C(O)—, —S—, —NH—, *—NH—C(O)—, or *-((L₁)-N)—C(O)-(L₂) wherein L₁ and L₂ indicates the bond to two carbons of a lactam cycle; most preferably —O— or —O—C(O)—.

In another preferred embodiment, Ra and Rb in formula (A1) or (A1′) independently from each other represent (C₁-C₅)alkyl, (C₂-C₅)alkenyl, a five- or six-membered heterocycle, (C₃₋₁₂)cycloalkyl, phenyl, optionally substituted with 1, 2, 3, 4 or 5 (C₁-C₅)alkyl, benzyl optionally substituted with 1, 2, 3, 4 or 5 (C₁-C₅)alkyl.

In another preferred embodiment, Ra and Rb in formula (A1) or (A1′) form together with the carbon they are attached to pyrrolidine, tetrahydrofuran, tetrahydrothiophene, pyrrole, furan, thiophen, piperidine, tetrahydropyran, tetrahydrothiopyran, pyridine, cyclopentyl or cyclohexyl.

In another preferred embodiment, Ra and Rb in formula (A1) or (A1′) represent together with the carbon they are attached to and V a (C₃₋₃₀)cycloalkyl moiety to which —W—Re is attached wherein the cycloalkyl moiety is selected from the group consisting of adamantyl (C₁₀H₁₄), iceanyl (C₁₂H₁₆), diadamantanyl (C₁₄H₁₈), triadamantyl (C₂₂H₂₂), isotetramantanyl (C₂₂H₂₆), pentamantanyl (C₂₆H₃₀), cyclohexamantanyl (C₂₆H₂₈), superadamantan (C₃₀H₃₄), each optionally substituted with 1, 2, 3, 4 or 5 substituents selected from the group consisting of hydroxy, halogen, (C₁-C₅)alkyl, (C₁-C₅)alkoxy; more preferably the cycloalkyl is selected from the group consisting of adamantyl (C₁₀H₁₄), iceanyl (C₁₂H₁₆), diadamantanyl (C₁₄H₁₈), triadamantyl (C₂₂H₂₂), isotetramantanyl (C₂₂H₂₆), pentamantanyl (C₂₆H₃₀), cyclohexamantanyl (C₂₆H₂₈), superadamantan (C₃₀H₃₄), more preferably the cycloalkyl is selected from the group consisting of adamantyl (C₁₀H₁₄), iceanyl (C₁₂H₁₆), diadamantanyl (C₁₄H₁₈), even more preferably adamantyl (C₁₀H₁₄)).

In yet another preferred embodiment, Ra and Rb in formula (A1) or (A1′) form together with the carbon they are attached to and V a phenyl moiety (—C₆H₄) to which —W—Re is attached.

In yet another preferred embodiment, Re represents a modified polymer (as defined herein).

Thus, one embodiment of the invention is directed to a UV light cleavable polymer/linker/drug conjugate. The conjugate comprises a polymer linked via an UV light cleavable diazirine group (linker) to a drug molecule. The UV light cleavable drug-polymer conjugate can be designed to function as a drug depot. The UV light cleavable drug-polymer conjugate is preferably formulated as a depot suitable for cutaneous, subcutaneous, intramuscular, intratumoral, intraorgan, in the vicinity of an organ, or brain implantation. Upon irradiation with UV light of a suitable wavelength, the UV light cleavable group (linker) is cleaved, thereby releasing the drug molecule from the polymer chain.

In one preferred embodiment, conjugates according to the invention can be used for emergency medication (e.g. provision of epinephrine, glucagon, etc.), local cancer therapy and injection free delivery of antibodies.

In yet another preferred embodiment, Re represents a modified polymer, Ra and Rb are independently selected from the group consisting of (C₁-C₅)alkyl, phenyl, benzyl; or Ra and Rb form together with the carbon they are attached to cyclopentyl or cyclohexyl; or Ra and Rb represent together with the carbon they are attached to and V a (C₃₋₃₀)cycloalkyl moiety to which —W—Re is attached wherein the cycloalkyl moiety is selected from the group consisting of adamantyl (C₁₀H₁₄), iceanyl (C₁₂H₁₆), diadamantanyl (C₁₄H₁₈), triadamantyl (C₂₂H₂₂), isotetramantanyl (C₂₂H₂₆), pentamantanyl (C₂₆H₃₀), cyclohexamantanyl (C₂₆H₂₈), superadamantan (C₃₀H₃₄), each optionally substituted with 1, 2, 3, 4 or 5 substituents selected from the group consisting of halogen, (C₁-C₅)alkyl, (C₁-C₅)alkoxy; more preferably the cycloalkyl is selected from the group consisting of adamantyl (C₁₀H₁₄), iceanyl (C₁₂H₁₆), diadamantanyl (C₁₄H₁₈), triadamantyl (C₂₂H₂₂), isotetramantanyl (C₂₂H₂₆), pentamantanyl (C₂₆H₃₀), cyclohexamantanyl (C₂₆H₂₈), superadamantan (C₃₀H₃₄), more preferably the cycloalkyl is selected from the group consisting of adamantyl (C₁₀H₁₄), iceanyl (C₁₂H₁₆), diadamantanyl (C₁₄H₁₈), even more preferably adamantyl (C₁₀H₁₄)); Rc and Rd each represent H; X represents a bond; and Y represents a moiety selected from the group consisting of *—O—, *—S—, *—NH—, *-((L₁)-N)—C(O)-(L₂) wherein L₁ and L₂ indicates the bond to two carbons of a lactam cycle of Rf, *—O—C(O)—, *—O—C(S)—, *—O—C(O)—NH—, *—S(O₂)—O—, *—O—S(O₂)—, *—O—C(S)—, *—NH—C(O)—, *—S(O₂)—NH—, and *—NH—S(O₂)—, *-triazol-, *—O—(C₂-C₅)alkyl-triazol-, *—O—C(O)—(C₂-C₅)alkyl-triazol-, *—C(O)O—(C₂-C₅)alkyl-triazol-, more preferably *—O—, *—O—C(O)—; *—O—C(O)—NH—, *—S—, *—NH—C(O)—, *—N((C₁-C₅)alkyl)-, *-((L₁)-N)—C(O)-(L₂) wherein L₁ and L₂ indicates the bond to two carbons of a lactam cycle of Rf, and *—NH—, even more preferably *—O—, *—O—C(O)—, —S—, —NH—, *—NH—C(O)—, and *-((L₁)-N)—C(O)-(L₂) wherein L₁ and L₂ indicates the bond to two carbons of a lactam cycle of Rf, *-triazol-, *—O—(C₂-C₅)alkyl-triazol-, *—O—C(O)—(C₂-C₅)alkyl-triazol-, *—C(O)O—(C₂-C₅)alkyl-triazol-, even more —O— or —O—C(O)—, or even more preferably, if Rf is a modified peptide, —O—. —O—C(O)—, *-triazol-, *—O—(C₂-C₅)alkyl-triazol-, *—O—C(O)—(C₂-C₅)alkyl-triazol-, *—C(O)O—(C₂-C₅)alkyl-triazol-.

One preferred embodiment refers to compounds of formula (A2), wherein X in Formula (A1) represents a bond and Rc and Rd each represent H:

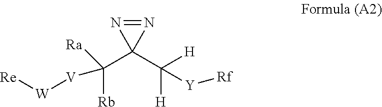

wherein Re, Rf, W, V, Ra and Rb are as defined in Formula (A1) and in any of the preferred embodiments of Formula (A1).

In one preferred embodiment, Ra and Rb in formula (A2) independently from each other represent a moiety selected from (C₁-C₅)alkyl, (C₂-C₅)alkenyl, a five- or six-membered heterocycle, (C₃₋₆)cycloalkyl (preferably cyclopropyl, cyclopentyl, cyclohexyl), phenyl, benzyl (—CH₂—C₆H₅) each heterocycle, cycloalkyl, phenyl or benzyl can optionally be substituted with 1, 2, 3, 4 or 5 substituents selected from the group consisting of (C₁-C₅)alkyl, (C₁-C₅)alkoxy; or
Ra and Rb form together with the carbon they are attached to a five- or six-membered heterocycle or (C₃₋₆)cycloalkyl (preferably cyclopropyl, cyclopentyl or cyclohexyl) optionally substituted with 1, 2, 3, 4 or 5 substituents selected from the group consisting of (C₁-C₅)alkyl, (C₁-C₅)alkoxy); or
Ra and Rb form together with the carbon they are attached to and V a cycloalkyl selected from the group consisting of adamantyl (C₁₀H₁₅), iceanyl (C₁₂H₁₇), diadamantanyl (C₁₄H₁₉), each optionally substituted with 1, 2, 3, 4 or 5 substituents selected from the group consisting of (C₁-C₅)alkyl, (C₁-C₅)alkoxy; or
Ra and Rb form together with the carbon they are attached to and V phenyl which is substituted by —W—Re; and
Y represents a moiety selected from the group consisting of *—O—, *—S—, *—NH—, *—N((C₁-C₅)alkyl)-, *-((L₁)-N)—C(O)-(L₂) wherein L₁ and L₂ are carbons of a lactame cycle of Rₑ or R_{f}, respectively, *—O—C(O)—, *—O—C(S)—, *—O—C(O)—NH—, *—S(O₂)—O—, *—O—S(O₂)—, *—O—C(S)—, *—NH—C(O)—, *—S(O₂)—NH—, and *—NH—S(O₂)—, *-triazol-, *—O—(C₂-C₅)alkyl-triazol-, *—O—C(O)—(C₂-C₅)alkyl-triazol-, *—C(O)O—(C₂-C₅)alkyl-triazol-, more preferably *—O—, *—O—C(O)—; *—O—C(O)—NH—, *—S—, *—NH—C(O)—, *—N((C₁-C₅)alkyl)-, *—NH—C(O)—, optionally wherein the amide function is part of a lactam, and *—NH—, even more preferably *—O— or, if Rf is a modified peptide, *-triazol-, *—O—(C₂-C₅)alkyl-triazol-, *—O—C(O)—(C₂-C₅)alkyl-triazol-, *—C(O)O—(C₂-C₅)alkyl-triazol-.

Especially preferred are those embodiments, wherein Ra and Rb do not represent hydrogen and Rc and/or Rd represent hydrogen. Such conjugates show improved regioselectivity of the break-down of the linker and thus, the yield of released drug, by avoiding the presence of hydrogen atoms at the alpha-position to the diazirine group on the Re-side of the diazirine group, the double bond which is formed during the breakdown of the diazirine group by irradiation is supposed to be formed on the Rf-side favoring the release of the free form of Rf or a free form derivative of Rf.

Of course, all other preferred embodiments for the conjugates of Formula (A1) in regard of V, W, Y, Re, Rf, Ra and Rb are explicitly mutatis mutandis also applicable for the preferred compounds of formula (A2).

For example, preferred conjugates of formula (A1) and (A2) are those of formulae E, F, G, H, I, J or K:

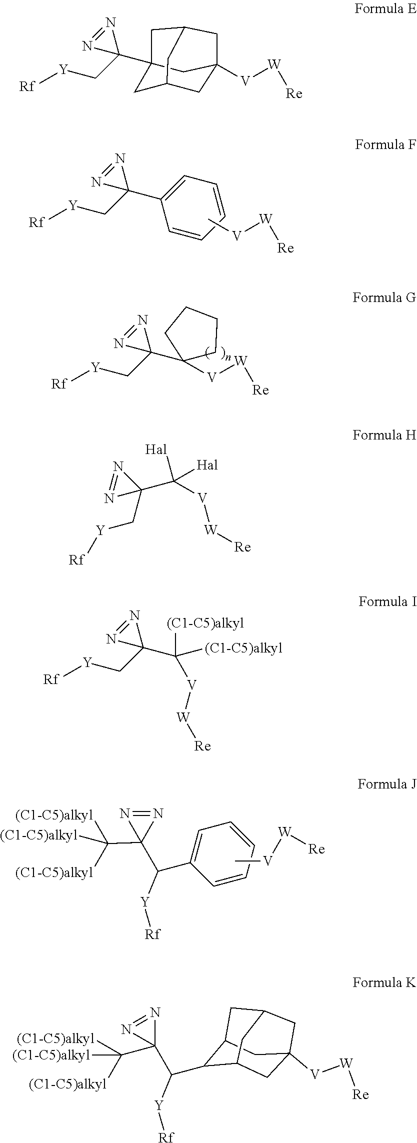

Wherein X represents a bond (and is, thus, not present in these preferred formulae), Re, Rf, Y, V, and W are as defined above, and Hal represents halogen, preferably Cl, F, Br, or I.

### Preferred Polymers

Examples of suitable polymers (free form of Re or Re′, respectively) for forming a conjugate according to the invention (and, thus, becoming a modified polymer as defined herein) include but are not limited to are polyacrylates (PA) (e.g., poly(hydroxyethylmethacrylate)), polymethacrylates (e.g. poly(hydroxyethylmethacrylate) (PHEMA)), polyacrylamides (e.g. poly(N-(2-hydroxypropyl)acrylamide)), polymethacrylamides (e.g. poly[N-(2-hydroxypropyl)]methacrylamide) (HPMA)), poly(alpha-hydroxy acids), poly(lactide-co-glycolides) (PLGA), polylactides (PLA), polyglycolides (PG), functionalized polystyrenes, polyethylene glycol (PEG), poly(alpha-hydroxy acids), polyorthoesters (POE), N-vinyl pyrrolidone, polyaspirins, polyphosphagenes, dendrimers, polyamides, proteins or peptides (e.g. albumin, collagen or fibrin), polysaccharides (e.g. cyclodextrine, agarose, starch, hyaluronic acid, chitosan, gelatin, or alginates) (in one more preferred embodiment selected from the group consisting of polyacrylates, polymethacrylamides, functionalized polystyrenes, polyamides, poly(alpha-hydroxy acids), PLGA, PLA, PG, PEG, poly(alpha-hydroxy acids), POE, dendrimers, polyaspirins, polyphosphagenes, dendrimers, proteins (e.g. albumin), peptides, and polysaccharides; in yet another preferred embodiment selected from the group consisting of polyacrylates, polymethacrylamides, functionalized polystyrenes and polyamides; such as PHEMA or HPMA; in yet another preferred embodiment selected from the group consisting of poly(alpha-hydroxy acids), PLGA, PLA, PG, PEG, poly(alpha-hydroxy acids), POE, dendrimers, polyaspirins, polyphosphagenes, dendrimers, proteins (e.g. albumin), peptides, polysaccharides; such as poly(alpha-hydroxy acids), PLGA, PLA, PG, PEG, poly(alpha-hydroxy acids), POE, albumin, collagen or fibrin, cyclodextrine, agarose, starch, hyaluronic acid, chitosan, gelatin, or alginates).

In one embodiment, a polymer is a functionalized polymer, i.e., a reactive group (which is not present in any of the afore mentioned polymers) selected from the group consisting of —N₃, ethynyl, ethenyl, a conjugated diene, isonitrile, and a tetrazine moiety, more preferably a reactive group selected from the group consisting of —N₃ and -ethynyl was attached to a polymer vie a reaction known in the art. There are a plethora of reactions that could be used to functionalize polymers such as esterification, amidation, alkylation, urea and thiourea formation, reductive amination, thio-esterification, transesterification, formation of disulfide crosslinks, nucleophilic aromatic substitution, silanation and among many others (as examples: Yang et al. Carbohydrate Polymers 84 (2011) 33-39). The skilled person is aware on the functional groups that exist on the surface of the used polymer as well as its chemical reactivity, therefore the approach on how to functionalize the polymer with clickable moieties can be chosen accordingly. For example, a polymer which contains multiple hydroxyl groups could be functionalized with a commercially available alkylating agent containing a terminal alkyne such as for instance propargyl bromide.

Further, it will be appreciated that naturally occurring or synthetic polymers in form of polypeptides can occur in either the L or D form (or a combination thereof), especially those containing large numbers of acidic (e.g., arginine, aspartic acid, glutamic acid) or basic side chains (e.g., lysine). For example, homopolypeptides of poly-L-lysine, poly-D-lysine, poly-L-ornithine, poly-L-glutamic acid, poly-L-arginine (hydrochloride), poly-D-glutamic acid, poly-D,L-glutamic acid, and poly-L-aspartic acid are commercially available from Alamanda Polymers (Huntsville, Ala.). Exemplary peptides are about 20, 50, 100, 200, 400, 600, or 800 amino acids in length or some range therebetween.

Most preferably, a polymer as used herein notably has no therapeutically activity, i.e. is not a drug as described herein. For example, the skilled person is well aware which polypeptides show a therapeutical activity (e.g. insulin) and which polypeptides show no therapeutically activity (e.g. poly-L-arginine).

In one preferred embodiment, the polymer is from the linear polyester family, such as polylactic acid, polyglycolic acid, or polycaprolactone and their associated copolymers, e.g., poly(lactide-co-glycolide) at all lactide to glycolide ratios, and both L-lactide or D,L lactide. Polymers such as polyorthoester, polyanhydride, polydioxanone, and polyhydroxybutyrate may also be employed.

In some aspects, the modified polymer in a conjugate according to the invention makes about up to 99.5%, 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, 70%, 65%, 60% or some range therebetween based on the total weight of a conjugate and the remainder is via the linker inactivated drug.

In another preferred embodiment, Re represents a modified drug. Preferably, Re and Rf represent the same modified drug.

Thus, one preferred embodiment of the present invention is directed to a UV light cleavable drug-drug conjugate. In such an embodiment, the UV light cleavable drug conjugate does not comprise a polymer that functions as a backbone for drug loading. Instead, the drug molecule is (cross)linked with UV light cleavable group(s) (linker(s)) to other drug molecule(s). The UV light cleavable drug conjugate is designed to function as a drug depot. The UV light cleavable drug conjugate is preferably formulated as a depot suitable for cutaneous, subcutaneous, intramuscular, intratumoral, intraorgan, in the vicinity of an organ, brain implantation. The preferred drug molecules are polymers having multiple functional groups suitable for crosslinking (for example, drug molecules containing one or more amine, amide hydroxy or carboxyl groups), such as therapeutic peptides. One preferred drug molecule is insulin. Upon irradiation with light of a suitable wavelength, the UV light cleavable group is cleaved, thereby releasing the drug molecule from the UV light cleavable drug conjugate.

In one preferred embodiment, Rf and Re each represent a drug, preferably the same drug, Ra, Rb, Rc and Rd each represent H, V and X are as defined in Formula (A1), preferably V and X each represent a bond, and W and Y independently from each other represent a moiety selected from the group consisting of *—O—, *—S—, *—NH—, *—N((C₁-C₅)alkyl)-*—C(O)—O—, *—O—C(O)—, *—O—C(O)—NH—, *—S(O₂)—O—, *—O—S(O₂)- *—C(O)—NH—, *-((L₁)-N)—C(O)-(L₂) wherein L₁ and L₂ are carbons of a lactam cycle of Rₑ or R_{f}, respectively, *—S(O₂)—NH—, *—NH—S(O₂)—, *-triazol-, *—O—(C₂-C₅)alkyl-triazol-, *—O—C(O)—(C₂-C₅)alkyl-triazol-, *—C(O)O—(C₂-C₅)alkyl-triazol-, (preferably wherein W and Y independently from each other represent *—O—, *—S—, *—O—C(O)—, *—N((C₁-C₅)alkyl)-, *-((L₁)-N)—C(O)-(L₂) wherein L₁ and L₂ are carbons of a lactam cycle of Rₑ or R_{f}, respectively, or *—NH—, even more preferably W and Y each represent *—O—).

In another preferred embodiment, Re (preferably a modified polymer or a modified drug, more preferably a modified polymer, more preferably selected from the group consisting of polyacrylates, polymethacrylamides, functionalized polystyrenes, polyamides, poly(alpha-hydroxy acids), PLGA, PLA, PG, PEG, poly(alpha-hydroxy acids), POE, dendrimers, polyaspirins, polyphosphagenes, dendrimers, proteins (e.g. albumin), peptides, and polysaccharides) is further modified by at least one further substitution of a reactive group of such a modified polymer or modified drug, wherein a further reactive group is substituted by a linker/Rf residue of formula (B)

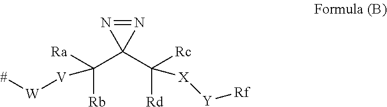

In an even more preferred embodiment, Re is a polymer selected from the group consisting of polyacrylates, polymethacrylamides, functionalized polystyrenes and polyamides; such as PHEMA or HPMA.

In yet another more preferred embodiment, Re is a polymer selected from the group consisting of poly(alpha-hydroxy acids), PLGA, PLA, PG, PEG, poly(alpha-hydroxy acids), POE, dendrimers, polyaspirins, polyphosphagenes, dendrimers, proteins (e.g. albumin), peptides, polysaccharides; such as poly(alpha-hydroxy acids), PLGA, PLA, PG, PEG, poly(alpha-hydroxy acids), POE, albumin, collagen or fibrin, cyclodextrine, agarose, starch, hyaluronic acid, chitosan, gelatin, or alginates).

In one more preferred embodiment, Re is a polymer and a polysaccharide. In one preferred embodiment, the polysaccharide is selected from the group consisting of cyclodextrine, agarose, starch, hyaluronic acid, chitosan, gelatin, or alginates, even more preferably selected from the group consisting of cyclodextrines, agarose, starch and gelantine preferably selected from the group.

In one preferred embodiment, a polymer as used for producing a conjugate according to the invention and/or a conjugate according to the invention and/or the altered polymer being the product of a UV-light initiated breakdown of a conjugate according to the invention, forms a solid or semi solid matrix.

In another preferred embodiment, a polymer as used for a conjugate according to the invention and/or a conjugate according to the invention and/or the altered polymer being the by-product of a UV-light initiated breakdown (e.g. a polymer still comprising part of the linker molecule at the position of the former reactive group of the unmodified polymer) of a conjugate according to the invention, is typically insoluble in the environment of implantation order to limit dispersal at the site of implantation. The polymer generally functions as a backbone for attachment of the drug molecule(s) via the UV light cleavable linker. When a conjugate according to the invention is used for implantation into a (human) body, one or more conjugates according to the invention form or are part of a depot.

In one preferred embodiment, the (modified or altered) polymer is biocompatible.

In another preferred embodiment, the (modified or altered) polymer is biodegradable.

In yet another preferred embodiment, the (modified or altered) polymer is bioresorbable.

In an even more preferred embodiment, the (modified or altered) polymer is biocompatible, biodegradable and bioresorbable.

A polymer suitable for forming conjugates according to the invention has at least one reactive group/functionality that will allow it to be joined to a linker group via said reactive group and a corresponding reactive group on a linker molecule. It will be appreciated that there are a wide variety of possible reactive groups that are possible in this regard. Exemplary reactive groups include, but are not limited to hydroxy, amine, carboxyl, (such as carboxylic acid, amide, carboxylic halide, carboxylic acid ester or carboxylic acid anhydride, and the carboxyl group may be activated, as is well known in the art, to facilitate coupling), thiocarboxylic acid (such as thiocarboxylic acid, thioamide, thiocarboxylic halide, thiocarboxylic acid ester or thiocarboxylic acid anhydride, and the thiocarboxyl group may be activated, as is well known in the art, to facilitate coupling), cyclic amide (lactame), vinylsulfone, alkyne, azide, maleimide, isothiocyanate, isocyanate, imidate, alpha-halo-amide, Michael acceptor, hydrazide, oxyamine, thiol, hydrazine, or a combination thereof. The reactive group may be located within the polymer chain (including as a side chain extending from the primary chain) and/or at the terminal end of the polymer chain. High loading of the drug molecules onto the polymer chain may be achieved when the reactive groups are located along the polymer chain.

In one preferred embodiment, a polymer suitable for forming a conjugate according to the invention comprises a carboxylic acid function (a carboxylic acid, a carboxy halide, an anhydride, an activated acid function etc.) as a reactive group. In such a case, the polymer can be linked to a linker molecule to form a conjugate according to the invention via a hydroxy group on the linker molecule. That is, the polymer forming the matrix is linked to the UV light cleavable linker via an ester bond. I. e. in one preferred embodiment, W in a conjugate according to the invention is —C(O)—O—* (wherein * indicates the bond to V).

In another preferred embodiment, a polymer suitable for forming a conjugate according to the invention comprises an amine functionality (reactive group). In such a case, the polymer can be linked to a linker molecule via a carboxylic acid function (a carboxylic acid, a carboxy halide, an anhydride, an activated acid function etc.) on the linker molecule. That is, the polymer forming the matrix is linked to the UV light cleavable linker via an amide bond. I.e. in one preferred embodiment, W in a conjugate according to the invention is —NH—C(O)—* (wherein * indicates the bond to V).

In another preferred embodiment, the polymer suitable for forming a conjugate according to the invention comprises an azide functionality. In such a case, the polymer can be linked to the UV light cleavable group via an alkyne on the linker molecule. That is, the polymer forming the matrix is linked to the UV light cleavable linker via a triazol bridge. For example, a polymer comprising a hydroxy group can be first reacted with HO(O)C—(C₁-C₅)alkyl-N₃ and said free form of the polymer can then react with a linker molecule comprising an alkyne-moiety in a click reaction. E.g., in one preferred embodiment, W in a conjugate according to the invention is a triazol moiety such as *-triazol-, *—O—(C₂-C₅)alkyl-triazol-, *—O—C(O)—(C₂-C₅)alkyl-triazol-(wherein * indicates the bond to V).

In still another embodiment, the polymer forming the matrix can have an alkyne functionality. In such a case, e.g., a polymer having a hydroxy group can be first reacted with HO(O)C—(C₁-C₅)alkyl-ethynyl and said free form of the polymer with the alkyne function can be reacted with a linker molecule having an azide function. E.g., in one preferred embodiment, W in a conjugate according to the invention is *-triazol-, *—O—(C₂-C₅)alkyl-triazol-, —O—C(O)—(C₂-C₅)alkyl-triazol (wherein * indicates the bond to V).

It will be appreciated that one or more UV light cleavable diazirine groups (or conjugates of a diazirine group and a drug) may be linked to a polymer. For example, in the case of chitosan, the polymer comprises a chain of glycosamine molecules such that multiple (i.e. more than one) amine functionalities (reactive groups) on the polymer may be each linked to a UV light cleavable group. In turn, this can provide for high loading of the drug molecules in such UV light cleavable drug-polymer conjugates. PEG, on the other hand is only functionalized on the end of a PEG chain. The skilled person will understand that due to steric reasons and other factors usually multiple but not all reactive groups of a polymer are linked to a UV light cleavable group. In case of agarose, the polymer comprises a chain of D-galactose and 3,6 anhydro-L-galactose molecules such that multiple (i.e. more than one) hydroxy functionalities (reactive groups) on the polymer may be each linked to a UV light cleavable group. Thus, the loading of a further modified polymer (or further modified drug) strongly depends on the nature and weight of the free form of the polymer, the structure of the diazirine group containing linker as well as the weight of the drug linked to the linker group. For example IgG (molecular weight of around 150 kDa) or Insulin (molecular weight of around 5.8 kDa) have much more weight than Epinephrine (molecular weight 182 Da).

The UV light cleavable drug conjugate of the present invention comprises one or more, preferably more than one, drug molecules each is bound to at least one linker via X or W, respectively. Thus, the “drug” that is UV light released from the conjugate may be a drug, drug precursor or modified drug that is not fully active or available until converted in vivo to its therapeutically active or available form. The drug may include small molecule compounds, peptides, proteins, or any other medicament or medicine used in the treatment or prevention of a disease or condition or is needed for diagnostic methods of the human body such as contrast agents, e.g. MRT probes or fluorophores, e.g., for controlling the release degree of a drug.

Representative non-limiting classes of drugs useful in the present invention include those falling into the following therapeutic categories: ACE-inhibitors; anti-anginal drugs; anti-arrhythmias; anti-asthmatics; anti-cholesterolemics; anti-convulsants; anti-depressants; anti-diarrhea preparations; anti-histamines; anti-hypertensive drugs; anti-infectives; anti-inflammatory agents; anti-lipid agents; anti-manics; anti-nauseants; anti-stroke agents; anti-thyroid preparations; anti-tumor drugs; anti-tussives; anti-uricemic drugs; anti-viral agents; acne drugs; alkaloids; amino acid preparations; anabolic drugs; analgesics; anesthetics; angiogenesis inhibitors; antacids; anti-arthritics; antibiotics; anticoagulants; antiemetics; antiobesity drugs; antiparasitics; antipsychotics; antipyretics; antispasmodics; antithrombotic drugs; anxiolytic agents; appetite stimulants; appetite suppressants; beta blocking agents; bronchodilators; cardiovascular agents; cerebral dilators; chelating agents; cholecystokinin antagonists; chemotherapeutic agents; cognition activators; contraceptives; coronary dilators; cough suppressants; decongestants; deodorants; dermatological agents; diabetes agents; diuretics; emollients; enzymes; erythropoietic drugs; expectorants; fertility agents; fungicides; gastrointestinal agents; growth regulators; hormone replacement agents; hyperglycemic agents; hypnotics; hypoglycemic agents; laxatives; migraine treatments; mineral supplements; mucolytics; narcotics; neuroleptics; neuromuscular drugs; NSAIDS; nutritional additives; peripheral vasodilators; polypeptides; prostaglandins; psychotropics; renin inhibitors; respiratory stimulants; steroids; stimulants; sympatholytics; thyroid preparations; tranquilizers; uterine relaxants; vaginal preparations; vasoconstrictors; vasodilators; vertigo agents; vitamins; wound healing agents; and contrast agents.

The preferred drugs molecules used in the present invention are those which are very potent such that they require relatively small amounts for the desired therapeutic effect but also need the blood levels to be carefully controlled. The preferred drugs are also those which benefit from good control of release.

In one embodiment, the drug molecule is a therapeutic peptide or protein, such as those described in US 2011/0166063 and U.S. Pat. No. 6,858,580. Preferred therapeutic peptides and proteins are selected from the group consisting of insulin; glucagon; calcitonin; gastrin; parathyroid hormones; angiotensin; growth hormones; secretin; luteotropic hormones (prolactin); thyrotropic hormones; melanocyte-stimulating hormones; thyroid-stimulating hormones (thyrotropin); luteinizing-hormone-stimulating hormones; vasopressin; oxytocin; protirelin; peptide hormones such as corticotropin; growth-hormone-stimulating factor (somatostatin); G-CSG, erythropoietin; EGF; physiologically active proteins, such as interferons and interleukins; superoxide dismutase and derivatives thereof; enzymes such as urokinases and lysozymes; and analogues or derivatives thereof. In another aspect, the therapeutic peptide or protein is selected from the group consisting of human growth hormone, bovine growth hormone, growth hormone-releasing hormone, an interferon, interleukin-1, interleukin-II, insulin, calcitonin, erythropoietin, atrial natriuretic factor, an antigen, an antibody, such as a monoclonal antibody, somatostatin, adrenocorticotropin, gonadotropin releasing hormone, oxytocin, vasopressin, analogues, or derivatives thereof.

### Preferred Drugs

Preferred drugs which can be modified according to the invention are generally all drugs having a hydroxyl, thiol, amine, amide or carboxyl group (which can be replaced by W or Y, respectively, in a conjugate according to the invention).

Usually, a free form of a drug, especially a registered pharmaceutical product in a national register of authorized medicines compiled by the European Medicines Agency (EMA) or respective other national registers such as of the USA, Australia and other countries in the world do not comprise an azide-, an alkyne-, a conjugated alkdiene-, an alkene-, an isonitrile- (isocyanide-), a tetrazine-moiety. However, the skilled person is well aware how to add such a functionality to a drug which is registered in a national register of authorized medicines, preferably a peptide or antibody, by standard reactions such as esterification, amidation, alkylation, urea and thiourea formation, reductive amination, thio-esterification, transesterification, formation of disulfide crosslinks and nucleophilic aromatic substitution. Such a functionalization can be achieved by methods well known in the art, for example, solid-phase peptide synthesis methods (Palomo et al. RSC Adv. 2014, 4, 32658-32672) and post synthesis modification (Bernardes et al. Chem. Rev. 2015, 115, 2174-2195) in the case of peptides and antibodies. In the case of non-peptides drugs, the skilled person is aware of the reactivity of the functional groups that exists in the drug that could be used for introducing a clickable moiety. The skilled person is well aware how to add such a functionality to a drug which is registered in a national register of authorized medicines, preferably a peptide or antibody, by standard reactions such as esterification, amidation, alkylation, urea and thiourea formation, thio-esterification, transesterification, formation of disulfide crosslinks and nucleophilic aromatic substitution. A skilled person is aware that clickable motifs, preferably azide and alkyne, and also conjugated alkadiene, alkene, isonitrile and tetrazine moiety, can be introduced in the drug (preferably peptide) during the solid-phase peptide synthesis methods by using a plethora of commercially available amino acids and other molecules comprising clickable groups (e.g., H-L-Cys(propargyl)-OH*HCl (CAS 3262-64-4 net), Propargyl-PEG(5)-COOH (CAS 1245823-51-1), Boc-L-Ser(propargyl)-OH*DCHA (CAS 145205-94-3), L-C-propargylglycine (CAS 23235-01-0) etc. (e.g. available by Iris Biotech, Aldrich and many other companies). This method allows introducing the clickable moiety in the exact amino acid sequence while the peptide is being assembled (synthesized by solid-phase). For example, a GLP-1-derivative which contains a terminal alkyne group could be obtained by using in the solid-phase synthesis, for instance, propargylglycine instead of any other amino acid of its natural sequence or extending its natural sequence with propargylglycine. The skilled person is aware that another way to introduce the mentioned groups in a molecule consists in post synthesis modifications. In post synthesis modification the drug (preferably peptide and antibody) is modified after it was synthesized or isolated from its natural environment. In the case of peptides, the preferred amino acids to be modified are the N-terminal and the C-terminal amino acids, as well as the side chains of lysine, cysteine, and tyrosine. The skilled person is aware of the chemical reactivity of these groups and the choice of the reaction to be performed upon each group. For example, the skilled person is aware that a terminal alkyne group could be introduced in a drug (preferably peptide) by post synthesis modification by alkylating a cysteine residue with an alkylating agent containing a terminal alkyne such as for instance propargyl bromide. In general, the main types of reaction in post synthesis modification are amidation, alkylation, urea and thiourea formation, esterification, thio-esterification, transesterification, formation of disulfide crosslinks and further common reactions which can be found in the literature (Bernardes et al. Chem. Rev. 2015, 115, 2174-2195). In the case of non-peptides drugs, the skilled person is aware of the reactivity of the functional groups that exists in the drug that could be used for introducing a clickable moiety. For example, the skilled person is aware that an azide could be introduced in the drug by acylating an existing amine (or hydroxyl) group with a commercially available carboxylic acid which contains an azide attached to it. For example, dopamine could be acylated with 4-azidobutyric acid in order to have a clickable moiety install on the chemical structure. In such a case, when a drug, preferably a peptide or antibody, comprises an azide-, an alkyne-, a conjugated alkdiene-, an alkene-, an isonitrile- (isocyanide-), a tetrazine-moiety as reactive complementary group to an thiol-, azide-, an alkyne-, a conjugated alkdiene-, an alkene-, an isonitrile- (isocyanide-), a tetrazine-moiety of a linker molecule (alone or which is via another reactive group already linked to a polymer or other drug) via a click reaction, the resulting compound—when the linker breaks down by applying UV-light—will be a free form derivative as described herein.

In one preferred embodiment, preferred drugs for a modified drug in a conjugate according to the invention (preferably Rf) are selected from the group consisting of a therapeutic peptide, an emergency drug, a cytotoxic agent, or an antibody.

In a more preferred embodiment, drugs for a modified drug in a conjugate according to the invention (preferably Rf) is a therapeutic peptide selected from the group consisting of glucagon and its derivatives, GLP-1 and its derivatives exendin, lixisenatide, liraglutide and semaglutide, Insulin and its short acting insulin derivatives insulin glulisine, insulin aspart and insulin lispro, and somatostatin analogue lanreotide.

In another more preferred embodiment, drugs for a modified drug in a conjugate according to the invention (preferably Rf) is an emergency drug selected from the group consisting of epinephrine, nitroglycerin, antihistamine (diphenhydramine), albuterol, salbutamol, glucagon, atropine, ephedrine, hydrocortisone, morphine, naloxone, lorazepam, midazolam, flumazenil, streptokinase.

In another more preferred embodiment, drugs for a modified drug in a conjugate according to the invention (preferably Rf) is a cytotoxic agent selected from the group consisting of Metransine, Maytansine derivatives vinblastine, vinorelbine, vindesine and ravtansine; Taxanes selected from the group consisting of paclitaxel and docetaxel; epothilone derivatives selected from the group consisting of ixabepilone, patupilone and utidelone; Cholchicine and cholchicin derivatives; Heat Shock Protein 90 inhibitors selected from the group consisting of radicicol, geldanamycin and 17-N-Allylamino-17-demethoxygeldanamycin; Topoisomerase Inhibitors selected from the group consisting of camptothecin and its analogues Atiratecan, Topotecan, Exatecan, Pegamotecan, Protecan and Irinotecan; Calicheamicin and its derivative n-acetyl-γ-calicheamicin); Kinase inhibitors selected from the group consisting of Monomethyl auristatin and Auristatin F-hydroxypropylamide; DNA minor groove binding alkylating agents selected from the group consisting of duocarmycin and its derivatives A, B1, B2, C1, C2 and DA, CC-1065, adozelesin, bizelesin, and carzelesin; DNA intercalator Doxorubicin, other cytotoxic agents selected from the group consisting of 5-fluorouracil and its derivatives Capecitabine and Tegafur; Temozolomide; Lenalidomide; and Pomalidomide.

In another more preferred embodiment, drugs for a modified drug in a conjugate according to the invention (preferably Rf) is an antibody selected from the group consisting of
Infliximab, Adalimumab, Ustekinumab, Omalizumab, Leronlimab, Dupilumab, Brolucizumab, Eptinezumab, Teprotumumab, Crizanlizumab, Satralizumab, Sarilumab, Tanezumab (e.g., suitable for immune and inflammatory disorders), Nivolumab, Pembrolizumab, Trastuzumab, Bevacizumab, Rituximab, Dostarlimab, Spartalizumab, Ublituximab (e.g. suitable for cancer therapy), Panitumumab, CanacinumabGolimumab, Ofatumumab, Denosumab, Belimumab, Ipilimumab, Ramucirumab, Nivolumab, Alirocumab, Daratumumab, Necitumumab, Evolocumab, Sekukinumab, Olaratumab, Atezulizumab, Avelumab, Brodalumab, Dupilumab, Durvalumab, Guselkumab, Sarilumab, Erenumab, Cemiplimab, Emapalumab, Maxetumomab pasodudax;
Even more preferably an antibody is selected from the group consisting of Infliximab, Adalimumab, Ustekinumab, Omalizumab, Leronlimab, Dupilumab, Brolucizumab, Eptinezumab, Teprotumumab, Crizanlizumab, Satralizumab, Sarilumab, Tanezumab, Nivolumab, Pembrolizumab, Trastuzumab, Bevacizumab, Rituximab, Dostarlimab, Spartalizumab, Ublituximab.

E.g., Example 2b demonstrates by preparing an intermediate conjugate with a (Boc protected) dopamine how to prepare a conjugate according to the invention for releasing catecholamines such as Epinephrine.

In one preferred embodiment, Rf is a modified Epinephrine.

In another preferred embodiment, Rf is a modified Insulin, Glucagon, or GLP-1.

In one embodiment, a drug is a functionalized drug, i.e., a reactive group (which is not present in any of the afore mentioned polymers) selected from the group consisting of —N₃, ethynyl, ethenyl, a conjugated diene, isonitrile, and a tetrazine moiety, more preferably a reactive group selected from the group consisting of —N₃ and -ethynyl was attached to a polymer vie a reaction known in the art. Such a functionalization can be achieved by methods well known in the art, e.g., by, for example, solid-phase peptide synthesis methods (Palomo et al. RSC Adv. 2014, 4, 32658-32672) and post synthesis modification (Bernardes et al. Chem. Rev. 2015, 115, 2174-2195) in the case of peptides and antibodies. In the case of non-peptides drugs, the skilled person is aware of the reactivity of the functional groups that exists in the drug that could be used for introducing a clickable moiety. The skilled person is well aware how to add such a functionality to a drug which is registered in a national register of authorized medicines, preferably a peptide or antibody, by standard reactions such as esterification, amidation, alkylation, urea and thiourea formation, reductive amination, thio-esterification, transesterification, formation of disulfide crosslinks and nucleophilic aromatic substitution. A skilled person is aware that clickable motifs, preferably azide and alkyne, and also conjugated alkadiene, alkene, isonitrile and tetrazine moiety, can be introduced in the drug (preferably peptide) during the solid-phase peptide synthesis methods by using a plethora of commercially available amino acids containing clickable groups. This method allows introducing the clickable moiety in the exact amino acid sequence while the peptide is being assembled (synthesized by solid-phase). For example, a GLP-1 derivative which contains a terminal alkyne group could be obtained by using in the solid-phase synthesis, for instance, propargylglycine instead of any other amino acid of its natural sequence or extending its natural sequence with propargylglycine. The skilled person is aware that another way to introduce the mentioned groups in a molecule consists in post synthesis modifications. In post synthesis modification the drug (preferably peptide) is modified after it was synthesized or isolated from its natural environment. In the case of peptides, the preferred amino acids to be modified are the N-terminal and the C-terminal amino acids, as well as the side chains of lysine, cysteine, and tyrosine. The skilled person is aware of the chemical reactivity of these groups and the choice of the reaction to be performed upon each group. For example, the skilled person is aware that a terminal alkyne group could be introduced in a drug (preferably peptide) by post synthesis modification by alkylating a cysteine residue with an alkylating agent containing a terminal alkyne such as for instance propargyl bromide. In general, the main types of reaction in post synthesis modification are amidation, alkylation, urea and thiourea formation, esterification, thio-esterification, transesterification, formation of disulfide crosslinks and further common reactions which can be found in the literature (Bernardes et al. Chem. Rev. 2015, 115, 2174-2195). In the case of non-peptides drugs, the skilled person is aware of the reactivity of the functional groups that exists in the drug that could be used for introducing a clickable moiety. For example, the skilled person is aware that an azide could be introduced in the drug by acylating an existing amine (or hydroxyl) group with a commercially available carboxylic acid which contains an azide attached to it. For example, dopamine could be acylated with 4-azidobutyric acid in order to have a clickable moiety installed on the chemical structure.

The skilled person will understand that conjugated according to the invention cannot only be used for immobilization of drugs suitable for humans but also for drugs which are suitable for animals such as productive live-stock such as cows or pigs or domestic animals such as cats and dogs.

In another embodiment, the drug molecule is an anti-diabetic agent already in the clinical practice or in the pipeline of development. The anti-diabetic drug molecules are broadly categorized herein as insulin/insulin analogs and non-insulin anti-diabetic drugs. The non-insulin anti-diabetic drugs may include, but not limited to, insulin sensitizers, such as biguanides (e.g., metformin, buformin, phenformin, and the like), thiazolidinedione (TZDs; e.g., pioglitazone, rivoglitazone, rosiglitazone, troglitazone, and the like), and dual peroxisome proliferator-activated receptor agonists (e.g., aleglitazar, muraglitazar, tesaglitazar, and the like). The non-insulin anti-diabetic drugs may also include, but not limited to, secretagogues, such as sulfonylureas (e.g., carbutamide, chlorpropamide, gliclazide, tolbutamide, tolazamide, glipizide, glibenclamide, gliquidone, glyclopyramide, glimepiride, and the like), meglitinides (e.g., nateglinide, repaglinide, mitiglinide, and the like), GLP-1 analogs (e.g., exenatide, liraglutide, albiglutide, taspoglutide, and the like), and dipeptidyl peptidase 4 inhibitors (e.g., alogliptin, linagliptin, saxagliptin, sitagliptin, vildagliptin, and the like). Further, the non-insulin anti-diabetic drugs may include, but not limited to, alpha-glucosidase inhibitors (e.g., acarbose, miglitol, voglibose, and the like), amylin analog (e.g., pramlintide and the like), SGLT2 inhibitors (e.g., dapagliflozin, remogliflozin, sergliflozin, and the like), benfluorex, and tolrestat.

In a preferred embodiment, the drug molecule is insulin. As used herein, the term insulin embraces analogues or derivatives thereof such as disclosed in US2011/0144010.

In a preferred embodiment, the carboxyl functionalities (reactive groups) found on insulin can form an ester with a hydroxy group of a linker molecule. Upon photolysis, the carboxyl functionality is released from the conjugate, generating native insulin. It will be appreciated that amine, hydroxy or other functional groups on insulin can also be used/can form a bond to a UV light cleavable diazirine linker. The same principles also apply to other peptides such as Glucagon and GLP-1 or any of the further peptides described herein.

Thus, in one preferred embodiment, the carboxyl functionalities (reactive groups) found on a peptide or antibody are reacted with a hydroxyl group of a linker molecule (W′ or Y′, respectively) to form an ester (W or Y, respectively).

Contrast agents are widely used in non-invasive imaging, in particular to diagnose cancers and abscesses. There are several types of imaging procedures conducted. In positron emission tomography (PET), two beta rays emitted from the decaying radionuclide are detected. In single photon emission computed tomography (SPECT), one beta ray emitted from the decayed radionuclide is detected. It has been found that PET provides a more exact location of the examined area, while SPECT is simpler and easier to use, and therefore used more often. Magnetic resonance imaging (MRI) is the use of a magnetic field instead of radiation to produce detailed, computer-generated pictures of organs, body areas, or the entire body. Magnetic particle imaging, a novel type of imaging technique, was invented by Philips Research, Hamburg. The basic principle is based on conventional magnetic resonance imaging (MRI). Computed tomography (CT) uses a sophisticated X-ray machine and a computer to create a detailed picture of the bodies, tissues and structures. Ultrasound (US) imaging employs ultrasonic soundwaves for generating such images. These techniques have in common that the examination of a patient is non-invasive and free of pain. They are therefore often used for preventive medical check-up as well as for the diagnosis of different disease patterns. Contrast agents are generally used to increase the sensitivity of the above-mentioned techniques. These contrast agents are employed to enhance the ability to distinct different areas of the examined tissue or body.

Examples of contrast agents for drug conjugates according to the invention are ¹⁸F-marked 2-fluoro-2-deoxy-glucose (¹⁸F-FDG), iodinated contrast agents such as iohexol, iodixanol and ioversol Gd³⁺ based metal complexes such as gadobutol, gadoterate, gadoteridol, gadopentetate, gadobenate, gadopentetic acid dimeglumine, gadoxentate, gadoversetamide, gadodiamide.

### Linker Molecule

A second aspect of the invention refers to a linker molecule of formula (D1)

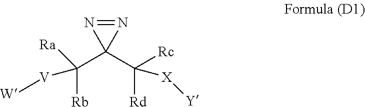

wherein
Ra and Rb independently from each other represents a moiety selected from H, halogen (preferably F, Cl, I or Br), (C₁-C₅)alkyl, (C₂-C₅)alkenyl, a five- or six-membered heterocycle, (C₃₋₃₀)cycloalkyl (preferably cyclopropyl, cyclopentyl, cyclohexanyl, adamantyl (C₁₀H₁₅), iceanyl (C₁₂H₁₇), diadamantanyl (C₁₄H₁₉), triadamantyl (C₂₂H₂₃), isotetramantanyl (C₂₂H₂₇), pentamantanyl (C₂₆H₃₁), cyclohexamantanyl (C₂₆H₂₉), superadamantan (C₃₀H₃₅)), phenyl, benzyl (—CH₂—C₆H₅) each heterocycle, cycloalkyl, phenyl or benzyl can optionally be substituted with 1, 2, 3, 4 or 5 substituents selected from the group consisting of hydroxy, halogen, (C₁-C₅)alkyl, (C₁-C₅)alkoxy; or
Ra and Rb form together with the carbon they are attached to a five- or six-membered heterocycle or (C₃₋₁₂)cycloalkyl, preferably cyclopentyl or cyclohexyl optionally substituted with 1, 2, 3, 4 or 5 substituents selected from the group consisting of hydroxy, halogen, (C₁-C₅)alkyl, (C₁-C₅)alkoxy); or
Ra and Rb represent together with the carbon they are attached to and V a (C₃₋₃₀)cycloalkyl moiety to which —W—Re is attached wherein the cycloalkyl moiety is selected from the group consisting of adamantyl (C₁₀H₁₄), iceanyl (C₁₂H₁₆), diadamantanyl (C₁₄H₁₈), triadamantyl (C₂₂H₂₂), isotetramantanyl (C₂₂H₂₆), pentamantanyl (C₂₆H₃₀), cyclohexamantanyl (C₂₆H₂₈), superadamantan (C₃₀H₃₄), each optionally substituted with 1, 2, 3, 4 or 5 substituents selected from the group consisting of hydroxy, halogen, (C₁-C₅)alkyl, (C₁-C₅)alkoxy; more preferably the cycloalkyl is selected from the group consisting of adamantyl (C₁₀H₁₄), iceanyl (C₁₂H₁₆), diadamantanyl (C₁₄H₁₈), triadamantyl (C₂₂H₂₂), isotetramantanyl (C₂₂H₂₆), pentamantanyl (C₂₆H₃₀), cyclohexamantanyl (C₂₆H₂₈), superadamantan (C₃₀H₃₄), more preferably the cycloalkyl is selected from the group consisting of adamantyl (C₁₀H₁₄), iceanyl (C₁₂H₁₆), diadamantanyl (C₁₄H₁₈), even more preferably adamantyl (C₁₀H₁₄)); or
Ra and Rb form together with the carbon they are attached to and V a phenyl moiety to which —W is attached;
Rc or Rd in formula represent H, the other space holder Rc or Rd, respectively, V and X are as defined in claims 1, 2 and 7 for the drug conjugate of formula (A1) and
W′ and Y′ are independently selected from a group consisting of hydroxy (—OH), thiol (—SH), amin (—NH₂), —Cl, —Br, —F or —I, a cyclic amide function (—N(H)—C(O)—) such as in a lactame, wherein the free bond of the nitrogen and the free bond of the C(O) carbon each represents a bond to a neighbored carbon atom of the cycle, sulfonic acid (—S(O₂)OH), —O—S(O₂)OH, thiocarboxylic acid (—C(S)OH), carboxyl (carboxylic acid (—C(O)OH)), a water soluble salt, such as a pharmaceutically acceptable salt, thereof, a carboxylic acid halide and anhydrates thereof), amide (—C(O)NH₂), carboxylic acid ester, preferably with (C₁-C₅)alkyl, isothiocyanate (—NCS), isocyanate (—NCO) such as —(C₁-C₅)—NCO, —NCO, —O—(C₁-C₅)—NCO, —O—C(O)—(C₁-C₅)—NCO, O—C(O)—NCO, C(O)O—NCO or —C(O)O—(C₁-C₅)NCO, hydrazine, a water soluble salt, preferably a pharmaceutically acceptable salt, of any of the foregoing, alkyne such as —O—(C₁-C₅)alkyl-ethynyl, —C(O)O—(C₁-C₅)alkyl-ethynyl or —O—C(O)—(C₁-C₅)alkyl-ethynyl, azide, such as —(C₁-C₅)alkyl-N₃, —N₃, —O—(C₁-C₅)alkyl-N₃, —O—C(O)—(C₁-C₅)alkyl-N₃ or —C(O)O— (C₁-C₅)alkyl-N₃, maleimide, imidate, alkenyl such as —O—(C₃—C)-alkenyl, —C(O)—O—(C₃—C)-alkenyl, —O—C(O)—(C₃—C)-alkenyl, a conjugated diene such as a conjugated —(C₄-C₁₀)alkdienyl, a conjugated —O—(C₅-C₁₀)alkdienyl, a conjugated —O—C(O)—(C₅-C₁₀)alkdienyl or —C(O)O—(C₅C₁₀)alkdienyl, tetrazinyl such as —(C₁-C₅)alkyl-tetrazinyl, -tetrazinyl, —O—(C₁-C₅)tetrazinyl, —O-tetrazinyl, —O—C(O)—(C₁-C₅)tetrazinyl, —O—C(O)— tetrazinyl, —C(O)O-tetrazinyl or —C(O)O—(C₁-C₅)tetrazinyl.

In one preferred embodiment,
Ra and Rb independently from each other represent (C₁-C₅)alkyl, (C₂-C₅)alkenyl, a five- or six-membered heterocycle, (C₃₋₁₂)cycloalkyl, phenyl, optionally substituted with 1, 2, 3, 4 or 5 (C₁-C₅)alkyl, benzyl optionally substituted with 1, 2, 3, 4 or 5 (C₁-C₅)alkyl.

Ra and Rb form together with the carbon they are attached to pyrrolidine, tetrahydrofuran, tetrahydrothiophene, pyrrole, furan, thiophen, piperidine, tetrahydropyran, tetrahydrothiopyran, pyridine, cyclopentyl or cyclohexyl.

Ra and Rb represent together with the carbon they are attached to and V a (C₃₋₃₀)cycloalkyl moiety to which —W′ is attached wherein the cycloalkyl moiety is selected from the group consisting of adamantyl (C₁₀H₁₄), iceanyl (C₁₂H₁₆), diadamantanyl (C₁₄H₁₈), triadamantyl (C₂₂H₂₂), isotetramantanyl (C₂₂H₂₆), pentamantanyl (C₂₆H₃₀), cyclohexamantanyl (C₂₆H₂₈), superadamantan (C₃₀H₃₄), each optionally substituted with 1, 2, 3, 4 or 5 substituents selected from the group consisting of hydroxy, halogen, (C₁-C₅)alkyl, (C₁-C₅)alkoxy; more preferably the cycloalkyl is selected from the group consisting of adamantyl (C₁₀H₁₄), iceanyl (C₁₂H₁₆), diadamantanyl (C₁₄H₁₈), triadamantyl (C₂₂H₂₂), isotetramantanyl (C₂₂H₂₆), pentamantanyl (C₂₆H₃₀), cyclohexamantanyl (C₂₆H₂₈), superadamantan (C₃₀H₃₄), more preferably the cycloalkyl is selected from the group consisting of adamantyl (C₁₀H₁₄), iceanyl (C₁₂H₁₆), diadamantanyl (C₁₄H₁₈), even more preferably adamantyl (C₁₀H₁₄)); or
Ra and Rb form together with the carbon they are attached to and V a phenyl moiety (—C₆H₄) to which —W′ is attached;
at least one of Rc and Rd represents H and the other substituent is selected from the group consisting of H, (C₁-C₅)alkyl, (C₂-C₅)alkenyl, a five- or six-membered heterocycle, (C₃₋₁₂)cycloalkyl, phenyl, optionally substituted with 1, 2, 3, 4 or 5 (C₁-C₅)alkyl, benzyl optionally substituted with 1, 2, 3, 4 or 5 (C₁-C₅)alkyl;
V and X are as defined in the conjugates of formula (A1) and
W′ and Y′ are independently selected from a group consisting of hydroxy, thiol, amine, carboxyl, (such as carboxylic acid, amide, carboxylic halide, carboxylic acid ester or carboxylic acid anhydride, and the carboxyl group may be activated, as is well known in the art, to facilitate coupling), thiocarboxylic acid (such as thiocarboxylic acid, thioamide, thiocarboxylic halide, cyclic amide (lactame), thiocarboxylic acid ester or thiocarboxylic acid anhydride, and the thiocarboxyl group may be activated, as is well known in the art, to facilitate coupling) vinylsulfone, alkyne, alkene, conjugated diene, azide, maleimide, isothiocyanate, isocyanate, imidate, alpha-halo-amide, Michael acceptor, hydrazide, oxyamine, thiol, hydrazine; preferably consisting of hydroxy (—OH), thiol (—SH), amin (—NH₂ or —N((C₁-C₅)alkyl)H—), sulfonic acid (—S(O2)OH), —O—S(O2)OH, thiocarboxylic acid (—C(S)OH), carboxyl (such as carboxylic acid (—C(O)OH), amide (—C(O)NH₂), carboxylic halide, preferably —C(O)Cl, carboxylic acid ester, preferably with (C₁-C₅)alkyl, or carboxylic acid anhydride), isothiocyanate (—NCS), isocyanate (—NCO), hydrazine, a water soluble salt of any of the foregoing, alkyne, preferably —O—(C₁-C₅)alkyl-ethynyl, —C(O)O—(C₁-C₅)alkyl-ethynyl or —O—C(O)—(C₁-C₅)alkyl-ethynyl, azide, preferably —O—(C₁-C₅)alkyl-N₃, —O—C(O)—(C₁-C₅)alkyl-N₃ or —C(O)O—(C₁-C₅)alkyl-N₃, maleimide, imidate; more preferably consisting of —OH, —SH, —NH₂, —C(O)OH, carboxylic halide, preferably —C(O)Cl, —S(O₂)OH, a water soluble salt of any of the foregoing, —O—(C₁-C₅)alkyl-N₃, —C(O)O—(C₂-C₅)alkyl-N₃, —O—C(O)—(C₁-C₅)alkyl-N₃, —O—(C₂-C₅)alkyl-ethynyl, —O—C(O)—(C₂-C₅)alkyl-ethynyl, and —C(O)O—(C₂-C₅)alkyl-ethynyl alkenyl such as —O—(C₃-C₇)-alkenyl, —C(O)—O—(C₃-C₇)-alkenyl, —O—C(O)—(C₃—C)-alkenyl, a conjugated diene such as a conjugated —(C₄-C₁₀)alkdienyl, a conjugated —O—(C₅-C₁₀)alkdienyl, a conjugated —O—C(O)—(C₅-C₁₀)alkdienyl or —C(O)O—(C₅C₁₀)alkdienyl, tetrazinyl such as —(C₁—C₅)alkyl-tetrazinyl, -tetrazinyl, —O—(C₁-C₅)tetrazinyl, —O-tetrazinyl, —O—C(O)—(C₁-C₅)tetrazinyl, —O—C(O)-tetrazinyl, —C(O)O-tetrazinyl or —C(O)O—(C₁-C₅)tetrazinyl.

All preferred embodiments for the conjugates of Formula (A1) in regard of X, V, Ra, Rb, Rc and Rd are explicitly mutatis mutandis also applicable for the linker molecules of Formula (D1).

In one more preferred embodiment, Y′ represents hydroxy (—OH), thiol (—SH), amin (—NH₂ or —N((C₁-C₅)alkyl)H), even more preferably hydroxy (—OH) or amin (—NH₂ or —N((C₁-C₅)alkyl)H—), —N₃, preferably —O—(C₁-C₅)alkyl-N₃, —O—C(O)—(C₁-C₅)alkyl-N₃ or —C(O)O—(C₁-C₅)alkyl-N₃, alkyne, preferably —O—(C₂-C₅)alkyl-ethynyl, —O—C(O)—(C₂-C₅)alkyl-ethynyl, or —C(O)O—(C₂-C₅)alkyl-ethynyl, most preferably hydroxy (—OH).

In yet another preferred embodiment, W′ represents hydroxy (—OH), amin (—NH₂ or —N((C₁-C₅)alkyl)H—), a carboxylic acid residue (—COOH), salt thereof, anhydride thereof or halide thereof or a click chemistry moiety, preferably selected from the group consisting of —O—(C₁-C₅)alkyl-N₃, —C(O)O—(C₂-C₅)alkyl-N₃, —O—C(O)—(C₁-C₅)alkyl-N₃, —O—(C₂-C₅)alkyl-ethynyl, —O—C(O)—(C₂-C₅)alkyl-ethynyl, and —C(O)O—(C₂-C₅)alkyl-ethynyl.

In yet another preferred embodiment, Y′ represents hydroxy (—OH), thiol (—SH), amin (—NH₂ or —N((C₁-C₅)alkyl)H), even more preferably hydroxy (—OH) or amin (—NH₂ or —N((C₁-C₅)alkyl)H—), most preferably hydroxy (—OH); and W′ represents hydroxy (—OH), amin (—NH₂ or —N((C₁-C₅)alkyl)H—), a carboxylic acid residue (—COOH), salt thereof, anhydride thereof or halide thereof, —O—(C₁-C₅)alkyl-N₃, —C(O)O—(C₂-C₅)alkyl-N₃, —O—C(O)—(C₁-C₅)alkyl-N₃, —O—(C₂-C₅)alkyl-ethynyl, —O—C(O)—(C₂-C₅)alkyl-ethynyl, or —C(O)O—(C₂-C₅)alkyl-ethynyl.

In yet another preferred embodiment, Y′ represents hydroxy (—OH), thiol (—SH), amin (—NH₂ or —N((C₁-C₅)alkyl)H—), even more preferably hydroxy (—OH) or amin (—NH₂ or —N((C₁-C₅)alkyl)H), most preferably hydroxy (—OH); and W′ represents hydroxy (—OH), amin (—NH₂ or —N((C₁-C₅)alkyl)H), a carboxylic acid residue (—COOH), salt thereof, anhydride thereof or halide thereof, —O—(C₁-C₅)alkyl-N₃, —C(O)O—(C₂-C₅)alkyl-N₃, —O—C(O)—(C₁-C₅)alkyl-N₃, —O—(C₂-C₅)alkyl-ethynyl, —O—C(O)—(C₂-C₅)alkyl-ethynyl, or —C(O)O—(C₂-C₅)alkyl-ethynyl; and Y′ and W′ each represent a different type of moieties.

One preferred embodiment refers to linker molecules of formula (D2), wherein X in Formula (D1) represents a bond and Rc and Rd represent each H:

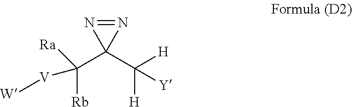

wherein W′, V, Ra, Rb and Y′ are as defined in Formula (D1) and in the preferred embodiments of Formula (D1).

All preferred embodiments for the conjugates of Formula (A1) in regard of, V, Ra and Rb are explicitly mutatis mutandis also applicable for the linker molecules of Formula (D1) (and (D2)).

Another aspect of the invention refers to the use of a linker molecule of formula (D1), preferably of formula (D2) to form a drug conjugate of formula (A1) or (A2), respectively.

Another aspect of the invention refers to a linker molecule of formula (D1′)

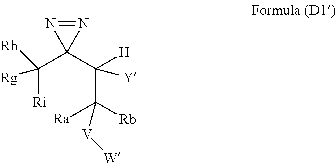

wherein
Ra, Rb, Rg, Rh, Ri and V are as defined in the conjugates of formula (A1′) and
W′ and Y′ are independently selected from a group consisting of hydroxy (—OH), thiol (—SH), amin (—NH₂ or —N((C₁-C₅)alkyl)H), halides such as Cl, Br, F or I, a cyclic amide function L₁-N(H)—C(O)-L₂, wherein the amide function is part of a lactam of Re and L₁ and L₂ represent carbons of the lactam cycle (i.e. wherein the bond of the nitrogen to L₁ and the bond of the C(O) carbon to L₂ each represents a bond to a neighbored carbon atom of the cycle, sulfonic acid (—S(O₂)OH), —O—S(O₂)OH, thiocarboxylic acid (—C(S)OH), carboxyl (carboxylic acid (—C(O)OH)), a water soluble salt, such as a pharmaceutically acceptable salt, thereof, a carboxylic acid halide and anhydrates thereof), amide (—C(O)NH₂), carboxylic acid ester, preferably with (C₁-C₅)alkyl, isothiocyanate (—NCS), isocyanate (—NCO) such as —(C₁-C₅)—NCO, —NCO, —O—(C₁-C₅)—NCO, —O—NCO, —O—C(O)—(C₁-C₅)—NCO, —O—C(O)—NCO, —C(O)O—NCO or —C(O)O—(C₁-C₅)NCO, hydrazine, a water soluble salt, such as a pharmaceutically acceptable salt, of any of the foregoing, alkyne such as —O—(C₁-C₅)alkyl-ethynyl, —C(O)O—(C₁-C₅)alkyl-ethynyl or —O—C(O)—(C₁-C₅)alkyl-ethynyl, azide, such as —(C₁-C₅)alkyl-N₃, —N₃, —O—(C₁-C₅)alkyl-N₃, —O—C(O)—(C₁-C₅)alkyl-N₃ or —C(O)O—(C₁-C₅)alkyl-N₃, maleimide, imidate, alkenyl such as —O—(C₃—C)-alkenyl, —C(O)—O—(C₃-C₇)-alkenyl, —O—C(O)—(C₃-C₇)-alkenyl, a conjugated diene such as a conjugated —(C₄-C₁₀)alkdienyl, a conjugated —O—(C₅-C₁₀)alkdienyl, a conjugated —O—C(O)—(C₅-C₁₀)alkdienyl or —C(O)O—(C₅C₁₀)alkdienyl, tetrazinyl such as —(C₁-C₅)alkyl-tetrazinyl, -tetrazinyl, —O—(C₁-C₅)tetrazinyl, —O-tetrazinyl, —O—C(O)—(C₁-C₅)tetrazinyl, —O—C(O)-tetrazinyl, —C(O)O—(C₁-C₅)tetrazinyl or —C(O)O-tetrazinyl.

In one preferred embodiment, Y′ represents hydroxy (—OH), thiol (—SH), amin (—NH₂ or —N((C₁-C₅)alkyl)H), halides such as Cl, Br, F or I, a cyclic amide function (—N(H)—C(O)—) such as in a lactame, wherein the free bond of the nitrogen and the free bond of the C(O) carbon each represents a bond to a neighbored carbon atom of the cycle, sulfonic acid (—S(O₂)OH), —O—S(O₂)OH, thiocarboxylic acid (—C(S)OH), carboxyl (carboxylic acid (—C(O)OH)), a water soluble salt, such as a pharmaceutically acceptable salt, thereof, a carboxylic acid halide and anhydrates thereof), amide (—C(O)NH₂), carboxylic acid ester, preferably with (C₁-C₅)alkyl, more preferably Y′ represents —OH.

All preferred embodiments for the conjugates of Formula (A1′) in regard of V, Ra, Rb, Rc, Rd, Rh, Ri, Rg are explicitly mutatis mutandis also applicable for the linker molecules of Formula (D1′).

### Alfa Carbonyl-Diazo Conjugates

Alternatively to a diazirine group in a conjugate according to the invention, also an alfa carbonyl-diazo conjugate can be used for releasing a drug in its free form or a free form derivative according to the present invention. A carbonyl-diazo conjugate according to the invention is, e.g., a conjugate of formula (E)

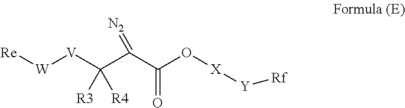

Wherein Re, W, V, X, Y and Rf are as defined for any of the drug conjugates above and R3 and R4 are independently selected from hydrogen or (C₁-C₅) alkyl.

### Modified Polymer

Another aspect of the present invention refers to a modified polymer Re, wherein the modification is the replacement of one or more, preferably more than one, reactive group(s) of such a polymer with a linker/drug conjugate of Formula (B)

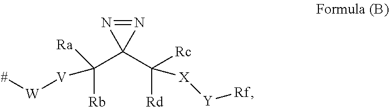

wherein # indicates the position of Re; and
wherein said one or more, preferably more than one, replaced reactive group of Re is selected from the group consisting of hydroxy (—OH), thiol (—SH), amin (—NH₂ or —N((C₁-C₅)alkyl)H), halides such as Cl, Br, F or I, L₁-N(H)—C(O)-L₂, wherein the amide function is part of a lactam of Re and L₁ and L₂ represent carbons of the lactam cycle, sulfonic acid (—S(O₂)OH), —O—S(O₂)OH, thiocarboxylic acid (—C(S)OH), carboxyl (carboxylic acid (—C(O)OH)), a water soluble salt, such as a pharmaceutically acceptable salt, thereof, a carboxylic acid halide and anhydrates thereof), amide (—C(O)NH₂), carboxylic acid ester, preferably with (C₁-C₅)alkyl, isothiocyanate (—NCS), isocyanate (—NCO) such as —NCO, —(C₁-C₅)—NCO, —O—(C₁-C₅)—NCO, —O—NCO, —O—C(O)—NCO, —O—C(O)—(C₁-C₅)—NCO, —C(O)O—(C₁-C₅)NCO or —C(O)O—NCO, hydrazine, a water soluble salt, such as a pharmaceutically acceptable salt, of any of the foregoing, ethynyl, azide, maleimide, imidate, ethenyl, a conjugated diene, tetrazinyl or a combination thereof; preferably said one or more, preferably more than one, replaced reactive group of Re is selected from the group consisting of —OH, —SH, —NH₂, —N((C₁-C₅)alkyl)H, a cyclic amide function (—N(H)—C(O)—) such as in a lactame, wherein the free bond of the nitrogen and the free bond of the C(O) carbon each represents a bond to a neighbored carbon atom of the cycle, —C(O)OH, —S(O₂)OH, a water soluble salt of any of the foregoing, azide, ethynyl or a combination thereof. For example, a polymer may comprise one or more carboxylic group(s) and one or more hydroxy groups and either one or more than one carboxylic groups, one or more hydroxy groups or a combination of one, some or all carboxylic groups and one, some or all hydroxy groups of such a molecule can be replaced by a linker/drug conjugate of formula (B).

All preferred embodiments for the conjugates of formula (A1) in regard of X, V, Y, W, Ra, Rb, Rc, Rd and Rf are explicitly mutatis mutandis also applicable for the modified polymers as described herein. In fact, a modified polymer is a conjugate according to formula (A1).

### Method of Preparing a Conjugate

For the final product, the modified polymer Re or a conjugate of formula (A1), it is irrespective if first the polymer/linker is formed to which then a drug is attached to the linker or if one or more linker/drug conjugates are formed, first and are then attached to a polymer.

Thus, another aspect of the present invention refers to a method of preparing a drug conjugate of formula (A1) or a modified polymer Re comprising the steps of
(1) reacting a linker molecule of formula (D1)

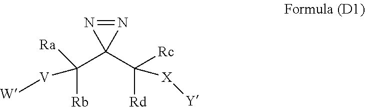

a) wherein W′ is a carboxylic group or a thiocarboxylic group via an esterification reaction of W′ with a —OH or —SH group, preferably a —OH group, of the free form of a polymer or drug; or wherein W′ is a —OH or —SH group, preferably a —OH group, via an esterification reaction of W′ with a carboxylic group or a thiocarboxylic group of the free form of a polymer or drug; or
b) wherein W′ is a carboxylic group or a thiocarboxylic group via an amid forming reaction of W′ with a —NH₂ or —N((C₁-C₅)alkyl)H group or a water soluble salt thereof of the free form of a polymer or drug Re; or wherein W′ is a —NH₂ or —N((C₁-C₅)alkyl)H group or a water soluble salt thereof via an amid forming reaction of W′ with a carboxylic group or a thiocarboxylic group of the free form of a polymer or drug; or
c) wherein W′ and the reactive group of the free form of a polymer or drug are suitable to perform a click reaction selected from the group consisting of Huisgen 1,3-dipolar cycloaddition, Thiol-ene reaction, Diels-Alder reaction [4+1] cycloadditions, e.g. W′ is a azide, thiol, substituted alkene or an isocyanide and the reactive group of the free form of a polymer or drug is an alkyne, an alkene, a conjugated diene or tetrazine, respectively; or
W′ is an alkyne, an alkene, a conjugated diene or tetrazine and the reactive group of the free form of a polymer or drug is an azide, thiol, substituted alkene or an isocyanide, respectively, e.g. the click reaction is a triazole forming click chemistry step and W′ is selected from the group consisting of —N₃, —O—(C₁-C₅)alkyl-N₃, —C(O)O—(C₁-C₅)alkyl-N₃, —O—C(O)—(C₁-C₅)alkyl-N₃ and the corresponding reactive group of a free form of a polymer or drug is selected from the group consisting of -ethynyl, —O—(C₁-C₅)alkyl-ethynyl, —O—C(O)—(C₁-C₅)alkyl-ethynyl, and —C(O)O—(C₁-C₅)alkyl-ethynyl or vice versa;
   and
(2) reacting Y′ of the resulting conjugate the resulting conjugate with a drug
   a) wherein Y′ is a carboxylic group or a thiocarboxylic group via an esterification reaction of Y′ with a —OH or —SH group, preferably a —OH group, of the free form of the drug; or wherein Y′ is a —OH or —SH group, preferably a —OH group, via an esterification reaction of Y′ with a carboxylic group or a thiocarboxylic group of the free form of the drug; or
   b) wherein Y′ is a carboxylic group or a thiocarboxylic group via an amid forming reaction of Y′ with a —NH₂ or —N((C₁-C₅)alkyl)H group or a water soluble salt thereof of the free form of a drug; or wherein Y′ is a —NH₂ or —N((C₁-C₅)alkyl)H group or a water soluble salt thereof via an amid forming reaction of W′ with a carboxylic group or a thiocarboxylic group of the free form of the drug; or
   c) wherein Y′ and the reactive group of the free form of the drug are suitable to perform a click reaction selected from the group consisting of Huisgen 1,3-dipolar cycloaddition, Thiol-ene reaction, Diels-Alder reaction [4+1]cycloadditions, e.g. Y′ is a azide, thiol, substituted alkene or an isocyanide and the reactive group of the free form of the drug is an alkyne, an alkene, a conjugated diene or tetrazine, respectively; or Y′ is an alkyne, an alkene, a conjugated diene or tetrazine and the reactive group of the free form of the drug is an azide, thiol, substituted alkene or an isocyanide, respectively, e.g. the click reaction is a triazole forming click chemistry step and Y′ is selected from the group consisting of —N₃, —O—(C₁-C₅)alkyl-N₃, —C(O)O—(C₁-C₅)alkyl-N₃, —O—C(O)—(C₁-C₅)alkyl-N₃ and the corresponding reactive group of a free form of the drug is selected from the group consisting of -ethynyl, —O—(C₁-C₅)alkyl-ethynyl, —O—C(O)—(C₁-C₅)alkyl-ethynyl, and —C(O)O—(C₁-C₅)alkyl-ethynyl or vice versa;
      wherein the order of step (1) and step (2) can be exchanged, optionally, the method can comprise additional reactive group protecting and deprotecting steps.

Preferred W′ and Y′ in a method of preparing a conjugate according to the invention are described in more detail for the linker molecule of formula (D1) and (D2).

The skilled person is aware how to choose the right reaction conditions and how to perform the required reactions without undue burden. Also, the skilled person is well aware if other reactive groups according to the invention are present in a polymer or drug or linker molecule, have to be protected according to well established and known protection reactions before reacting another reactive group of any of the afore mentioned molecules. Also the deprotection of such reactive groups is well known to the skilled person.

Preferably, if preparing a conjugate according to the invention a reactive group
—OH in an unmodified drug Rf is replaced by Y representing *—O—, e.g. by reacting a —O—S(O₂)OH group with a —OH group; or reacting a halide such as —I or —Br with the —OH group in an unmodified drug;
—SH in an unmodified drug Rf is replaced by Y representing *—S—;
—COOH, a pharmaceutically acceptable salt thereof, a water-soluble salt thereof, a halide derivative thereof or an anhydride thereof in an unmodified drug Rf is replaced by Y representing *—O—C(O)—, e.g., by reacting a —OH group with a —COOH group (or an activated from thereof), to form the ester;
—NH₂ in an unmodified drug Rf is replaced by Y representing *—NH—,
—NH₂ in an unmodified drug Rf is replaced by Y representing *—O—C(O)—NH—, e.g., by reacting a —NH₂ group with a —OH group in the presence of carbonyl diimidazole;
—N((C₂-C₅)alkyl)H in an unmodified drug Rf is replaced by Y representing *—N((C₁-C₅)alkyl)-,
—CONH₂ in an unmodified drug Rf is replaced by Y representing *—NH—C(O)—, e.g. via a transamidation reaction with the respective amine-group.
—S(O₂)OH, a pharmaceutically acceptable salt thereof, a water soluble salt thereof, a halide derivative thereof or an anhydride thereof in an unmodified drug Rf is replaced by Y representing *—O—S(O₂)—
the amide function of a cyclic amide —N(H)—C(O)— in an unmodified drug Rf is replaced by Y representing *—N(C(O)—)—, e.g., by reacting a halide such as —I with the cyclic amid function —N(H)—C(O)—.
a —N₃ group in an unmodified drug Rf is replaced by a triazole group, e.g. by reacting a ethynyl with the —N₃ moiety.

The skilled person is aware that the triazole reaction of an azide and an alkyne can result in two isomers (see Scheme A):

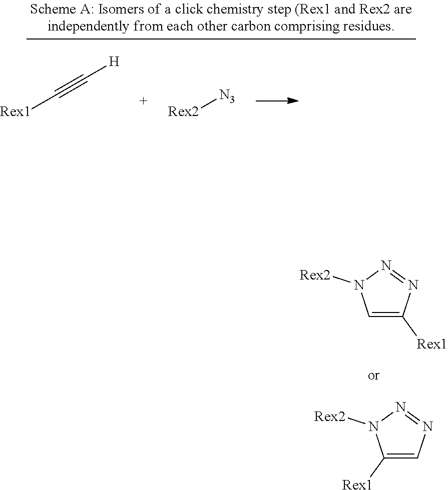

Depending on the condition and substituents Rex1 and Rex2, the reaction may result in one of the isomers or a mixture of the two isomers. When reference of W, W′, Y and Y′ is made to triazole residues, all three potential outcomes are encompassed. For example, Rex1 is a modified Rf and Rex2 is X in a compound of any of the formulae disclosed herein; or Rex1 is V and Rex2 is a modified Re in any formulae disclosed herein.

Thus, in one preferred embodiment, when W or Y, respectively, stands for a triazole moiety, the term triazole refers to the first isomer in Scheme 1:

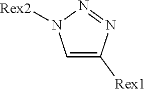

or a combination of the two isomers, wherein the amount of isomer 1 is at least 90% or higher, preferably 99% or higher based on the amount of the two isomers.

In another preferred embodiment, when W or Y, respectively, stands for a triazole moiety, the term triazole refers to the second isomer in Scheme 1:

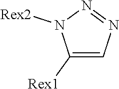

or a combination of the two isomers, wherein the amount of isomer 1 is at least 90% or higher, preferably 99% or higher based on the amount of the two isomers.

In yet another preferred embodiment, when W or Y, respectively, stands for a triazole moiety, the term triazole refers to a mixture of isomer 1 and isomer 2, wherein the amount of both isomers is below 90% and the total sum of isomer 1 and isomer 2 is 100%.

One further aspect of the present invention refers to the use of a compound of formula (D1) or (D2) in the production of a conjugate of the present invention.

### Use of Linker

Another aspect of the present invention refers to the use of a diazirine group for releasing an immobilized drug from a drug conjugate according to formula (A1) by applying UV light to the diazirine group wherein the UV-light is in a range between 100 nm and 400 nm.

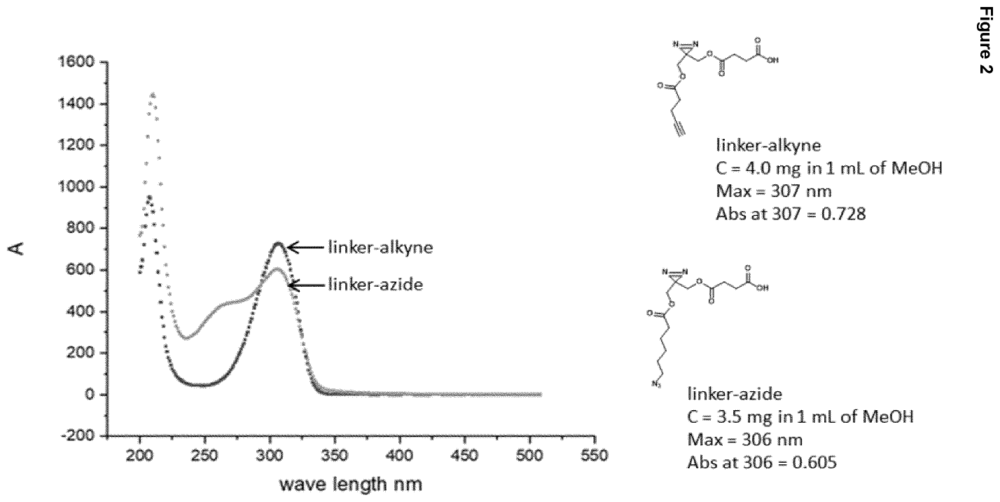

The skilled person is aware that the typical diazirine chromophore has a maximum absorption around 310 nm to 340 nm such as between 300 nm and 370 nm. The higher nm absorption ranges such as between 340 nm and 370 nm may occur, e.g. in the presence of aromatic groups. Typically, the range is between 300 and 350 nm. Usually, there is no absorption of such diazirine compounds above 370 nm. However, the skilled person can easily detect the right range of UV-light to be used to cleave a drug conjugate according to the invention by measuring the UV-Vis spectrum of a conjugate of the invention (e.g. within the range of, e.g., 175 to 550 nm, e.g., by using a Nanodrop-2000, Nanodrop, USA as it was also used in the experiments disclosed herein (see experimental section). Exemplarily, FIG. 2 shows the UV-Vis spectrum of two linker molecules according to the invention in methanol. When identifying the range of absorption, the skilled person can choose a light source suitable to emit light in a range, which is suitable to cleave the diazirine linker according to the invention.

Thus, in one preferred embodiment of the use of a diazirine group for releasing an immobilized drug from a drug conjugate according to the invention by applying UV light to the diazirine group, the UV-light is in a range between 250 nm and 360 nm preferably in a range between 260 and 350 nm.

The skilled person will understand that the spectrum of a UV-light source may comprise a broader spectrum then between, e.g., 250 nm and 360 nm (or 260 nm and 350 nm) or may only cover a part of such a range, but as long as a source provides at least partly light with a sufficient intensity within the disclosed ranges, such a source can be used to cleave the conjugates according to the invention. The skilled person is aware how to determine if a UV-light source as well as if the intensity of a UV-light source is suitable to cleave a linker of the invention by measuring release of a free form by standard analytic steps known in the art, depending on the drug to be released e.g. MS, HPLC, NMR, IR or fluorescence spectroscopy etc.

Another aspect of the invention refers to the use of a linker molecule according to the invention for inactivating a drug.

Another aspect of the invention refers to the use of a linker molecule according to the invention to immobilize a drug on a polymer.

A drug conjugate for use in medicine, wherein the drug conjugate comprises a diazirine group for releasing an immobilized drug from a drug conjugate by applying UV light to the diazirine group.

### Method of Use of a Linker

Another aspect of the present invention refers to a method of releasing a drug from a drug conjugate according to the invention by applying UV-light with a wave length in a range between 100 nm and 400 nm, more preferably in a range between 250 and 360 nm, most preferably in a range between 260 nm and 350 nm to said drug conjugate according to the invention.

Another aspect refers to a method of releasing a drug by applying light with a wave length in a range between 100 nm and 400 nm, more preferably in a range between 270 and 400 nm, most preferably in a range between 300 nm and 390 nm to modified polymer as described herein.

Especially preferred are conjugates of formula (A2). When applying UV-light to the linker of a conjugate according to formula (A2), the breakdown of the diazirine linker produces as by-product nitrogen gas, and the free form of a drug and, e.g., an aldehyde.

Exemplarily, Scheme 1 shows a UV-light induced breakdown of a conjugate according to the invention in case, e.g. Rc and Rd each represent H, X represents a bond and —Y—Rf represents *—O—Rf, or *—O—C(O)—Rf. In these examples, the restored reactive group of Rf is —OH or —COOH, respectively.

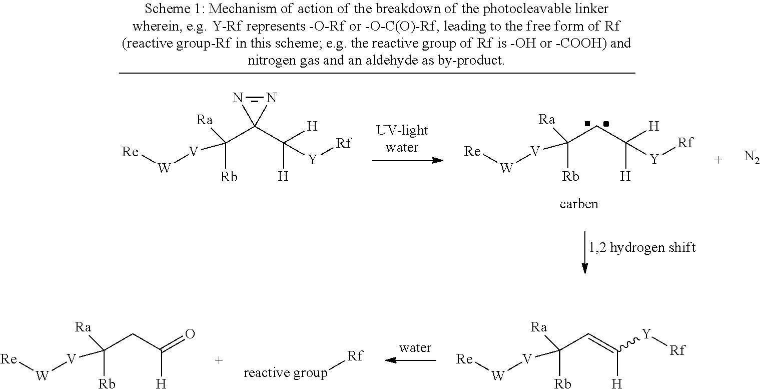

Based on the properties of diazirine linkers, the conjugates of the present invention selectively “collapses” upon applying a light trigger to rapidly release the desired drug.

In this process, the diazirine-system, after absorption of UV light, forms nitrogen gas and a carbene, which then undergoes a double-bond formation through a rapid 1,2 hydrogen shift. The resulting labile double-bond is readily cleaved in protic solvents such as water, to release the drug molecule. The second step does proceed quickly, however, its kinetics can be adjusted by choice of appropriate reactive group of the drug moiety as well as Ra, Rb, Rc and/or Rd. The used diazirines are easily and inexpensively produced, cleavage will only release nitrogen in equimolar amounts in regard of the cleaved linkers.

### Depot

Another aspect of the invention refers to a depot for controlled release of a drug, which is suitable to be implanted into a patient comprising a conjugate according to the invention. In one preferred embodiment, such a depot consists of one or more conjugates according to the invention. In another preferred embodiment, such a depot consists of a modified polymer of the invention.

For such depots, in one embodiment, irradiation is accomplished by a light source located external to the patient. The external light source may be possibly worn like a band, patch, or bandage over the depot site. In such embodiment, the external light source may also serve as a shield from ambient light. The irradiation to promote photo release of the drug can be provided by a variety of sources including, but not limited to light emitting diodes (LEDs), lasers, pens, fluorescence, or ultraviolet bulbs. Various phototherapy devices are known in the art and could be readily adapted to emit UV light of the respective wavelength for use in the present invention. For example, there are many commercially phototherapy devices uses for the treatment of psoriasis, wound repair, and other skin diseases (such as those manufactured by TheraLight, Inc.) which could be modified for use in the present invention.

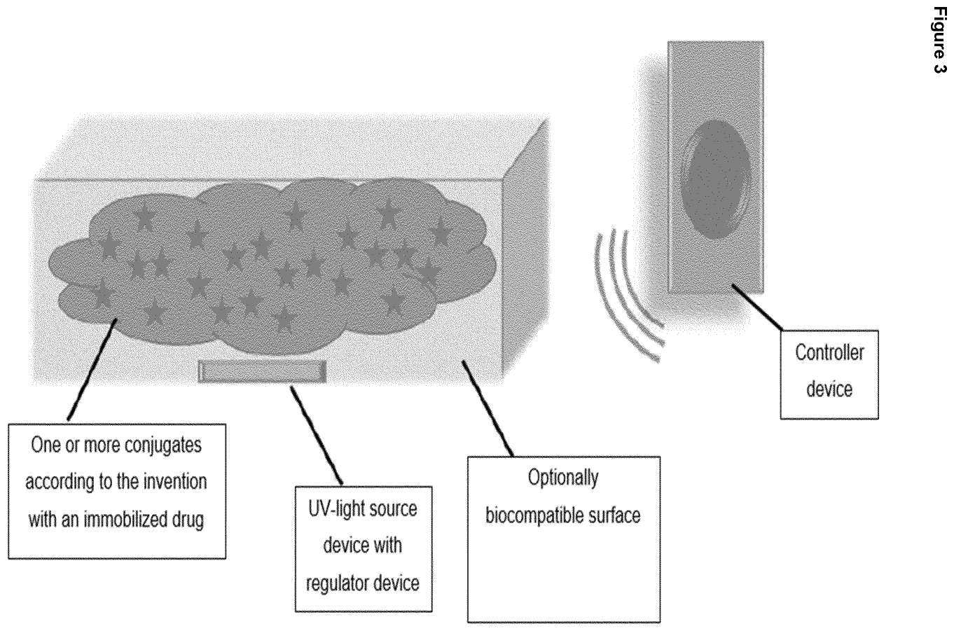

Another preferred embodiment refers to a depot suitable for implantation into a patient comprising one or more conjugates according to the invention a UV-light source device suitable to cleave the diazirine linker in a conjugate according to the invention (or a modified polymer according to the invention), a regulator device for the light source which can be wirelessly (e.g. by radiocommunication) connected with a controller device. Optionally, the controller device, e.g. a computer programmed to initiate and regulate and terminate irradiation of UV-light of the UV-light source device in the depot, is also comprised in the depot and can, thus, replace optionally the regulator device by taking over its function. FIG. 3 provides a general concept of use for a depot according to the invention with a remote control device which is wirelessly connected with a regulator device/UV-light source device in the depot.

Upon exposure to light of the appropriate wavelength, the drug molecule is cleaved from a drug conjugate according to the invention/a modified polymer according to the invention via UV-light (photolysis), thereby releasing the drug from the conjugate. The desired drug release from the conjugate may also be modulated by controlling the intensity of the light exposure, duration of the light exposure, and the location of implantation.

In case the controller device is not comprised in the depot, the emission of UV-light by the UV-light source device can be activated/controlled/deactivated via a controller device which is wirelessly, e.g. vie radiocommunication, connected to the regulator device of the light source.

In one preferred embodiment, the UV-light source device is a LED (light emitting diode), more preferable a LED being able to emit UV-light with a wavelength in the range between 100 nm and 400 nm, more preferably in a range between 250 and 360 nm, most preferably in a range between 260 nm and 350 nm.

UV light emitting devises are known in the art, e.g. an implantable, wireless blue light emitting diode (peak wavelength: 410 nm) (Zhang et al, Photobiomodulation, Photomedicine, and Laser Surgery Vol. 38 No. 11, 2020, pp) or an implantable, wireless LED (Nakajima et al., Oncotarget, 2018, Vol. 9, No. 28, pp 20048-20057). The skilled person is able to construct an LED with the required characteristics (size, emitted wave length) without undue burden.

In one preferred embodiment, the regulator device is a computer programmed to initiate and regulate and terminate irradiation, such as a computer ship with an internal power supply.

Generally, the size (volume of the UV-light source and its control device) should be as small as possible. In one embodiment, the maximum volume expansion of the UV-light source device in its longest elongation including the regulator device is 15 mm or less, such as between 15 mm and 2 mm or between 12 mm and 5 mm. However, also other sizes/volumes are suitable, depending on the intended treatment. A UV-light source and/or its control device can be covered by a biocompatible transparent surface such as a biocompatible transparent epoxy resin.

In another preferred embodiment, a depot further comprises at least a biocompatible, for the drug permeable but not for the polymer permeable surface material, e.g. a cage (e.g. a metal alloy or a ceramic) wherein the holes are big enough to allow released drug molecules (or free form derivative molecules) to leave the cage but small enough so that the polymer residue (after a UV induced breakdown of a linker) is retained within the cage. The cage can have any form such as asymmetrically shaped, spherically shaped or cubical shaped. A cage should be comprised of biocompatible and most preferred biocompatible and biostable materials such stainless steel materials, e.g., cobalt-chromium alloy, ceramic materials such as bioglass, alumina or hydroxypapatite, polymers such as medical grade silicone, PVC, PE or PP. A surface may also comprise or consists of a membrane material which allows a released drug to pass through while the polymerresidue is retained. One can also combine a cage like surface with a membrane surface.

In one embodiment, the depot or the controller device optionally comprises one or more sensors for measuring parameters of interest (such as blood glucose) in a patient. Such devices are generally described in, e.g. US 2009/0054750; US 2009/0164239; US 2008/0172031; US 2005/0065464. The skilled person is able to construct required sensors with the required characteristics (size, parameter to be measured) without undue burden. The information provided by the sensor trigger via the controller device (being either part of the depot or being an external controller device being wirelessly connected to the regulator device (e.g. via radiocommunication)) and the regulator device the activation/control/deactivation of the light source to release sufficient drug to correct the parameter of interest in a patient.

For example, drug in a depot may be released by irradiation in response to a physiological signal. For example, when the drug is insulin, blood sugar information provided by the patient through traditional finger sticks or by one of the non-invasive monitoring methods being developed in the field of use or sensor(s) implanted into a patient or being part of a depot according to the invention can be used. Alternative biomolecules may be (stabilized) glucagon derivatives or GLP-1 derivatives.

The depot comprising or being wirelessly in contact with a controller device such as a computer programmed to initiate irradiation of UV-light can release a drug in several different ways. The irradiation may be provided at fixed or variable intervals. For example, for drugs requiring conventional twice per day (“BID”) or three times per day (“TDD”) dosing, the light emitting device may be programmed to provide irradiation two or three times per day, respectively. Alternatively, the light source may be coupled via the regulator device or the controller device to a sensor which measures a parameter dependent upon the drug concentration in the body and then provides feedback to the light emitting device to control the light irradiation. For example, in the case of insulin, the UV-light source device may be coupled to a sensor which measures the amount of insulin in the blood stream or other parameter (most likely the blood glucose concentration). The UV-light source device may be programmed to irradiate the depot in a patient in accordance with that feedback loop. In short, the amount of light generated from the light emitting device can be periodically or continually modulated depending on the desired outcome. Sensors and other devices for measuring the dependent variable of interest (such as blood glucose) are generally described in, e.g. US 2009/0054750; US 2009/0164239; US 2008/0172031; US 2005/0065464.

The UV light cleavable drug conjugate of the present invention may provide immediate release of the drug, sustained release of the drug, or a combination thereof. For example, in general, immediate release of the drug may occur by irradiation of the UV light cleavable drug conjugate with appropriate light such that the drug is released from the UV light cleavable drug conjugate. This generally results into the introduction of the active drug into the body and that such that the drug is allowed to dissolve in or become absorbed at the location to which it is administered, with little or no delaying or prolonging of the dissolution or absorption of the drug.

As another example, once cleaved from the UV light cleavable drug conjugate, the drug may also undergo sustained release. In general, sustained release (also referred to as extended release or controlled release) encompasses ability of the UV light cleavable drug conjugate to continuously or continually release of the drug over a predetermined time period as a result of controlled irradiation with light. That is, the depot comprising UV light cleavable drug conjugate comprises a reservoir of drug molecules in which the release of the drug molecules from the conjugate may be photo controlled over an extended period of time (e.g., days, weeks, or months).

In one aspect, the present invention overcomes the problem associated with conventional drug delivery whereby frequent injections of the drug, such as insulin, are needed. For example, a patient may require a total daily dose of insulin of about 1 to 100 IU per day (e.g., about 1, 2, 3, 4, 5, 6, 7, 8, 9, 10, 20, 30, 40, 50, 60, 70, 80, 90, 100 IU per day), and typically about 0.1 to 2 IU/kg/day (e.g., about 0.1, 0.2, 0.3, 0.4, 0.5, 0.6, 0.7, 0.8, 0.9, 1, 1.0, 1.1, 1.2, 1.3, 1.4, 1.5, 1.6, 1.7, 1.8, 1.9, 2 IU/kg/day). This may be a dose of about 1 to 4 mg of insulin per day. In the present invention, the depot may contain a supply of insulin that lasts for several days, weeks, or even months, including a supply for 2, 3, 4, 5, 6, 7, 8, 9, 10, 11, 12, 13, 14, 15, 20, 25, 30, 35, 40, 45, 50, 55 or 60 days. It is contemplated that in one aspect, an entire one-month, or even two-month, supply or more of insulin could be deposited in the drug depot in a single injection in a volume equivalent to a single dose of traditional insulin. This dramatically reduces the number of injections needed to control a patient's disease, that is, there may be as much as a 50-, 100-, or even 200-fold reduction in the injection number. In another aspect, the present invention overcomes the problem associated with conventional insulin use whereby there is significant variability of blood sugar levels. In the present invention, there is a potential for rapid (e.g., real time, minute-by-minute, or hour-by-hour) correction of blood sugar levels through the non-invasive and continuously variable release of insulin with light. In one aspect, normalized non elevated blood sugar levels comparable to non-diabetic could potentially be obtained.

Further, when the drug molecule is insulin, there is a potential for rapid (e.g., real time or even minute by minute) correction of blood sugar levels through the non-invasive and continuously variable release of insulin with UV light. In one aspect, native like, rock-level blood sugar levels of a non-diabetic could potentially be obtained.

The new delivery system represents a flexible platform that allows efficient liberation of a native drug. UV light is used, which is not penetrating the skin and which will be generated within the depot. Unlike other triggers (magnetic waves, ultrasound), this ensures delivery with high precision and eliminating the danger of liberation by outside influences, which is of utmost importance to ensure the safety of the patient.

### Method for Administering a Drug

Another aspect of the invention refers to a method of administering a drug to a patient comprising:
implanting a depot or conjugate of the invention into a patient;
activating the UV light source through radiocommunication to emit light sufficient to cleave said linker group and release said drug molecule from the UV light cleavable drug-polymer conjugate.

Another aspect of the invention refers to a system for administering a drug to a patient comprising:
a depot comprising a UV light cleavable conjugate according to the invention and a UV-light source device, a regulator device coupled with the UV-light source device and a controller device wherein the controller is wirelessly connected to the regulator device. In one preferred embodiment, the controller device is a remote control. In another preferred embodiment, the controller device comprises a or receives data from a sensor for measuring a biological parameter wherein a specific value of said parameter initiates the release of drug from said conjugate. Another preferred embodiment refers to said system wherein said controller device is programmed to provide UV-light via the UV-light source device in response to a biological parameter in a patient and wherein said system further comprises a sensor for measuring said biological parameter to provide feedback to said controller device controlling the UV-light emission of the UV-light source device (directly or via a wirelessly connected regulator device).

Another aspect refers to a method of administering a drug to a patient comprising: implanting a depot into a patient;
transdermally irradiating said implanted depot with UV light sufficient to cleave said UV light cleavable group and release said drug molecule from the UV light cleavable drug conjugate.

Another aspect refers to a drug conjugate or a depot as described above for use in a method of administering a drug to a patient comprising: implanting a drug conjugate or a depot as described above into a patient; activating a UV light source to emit light with a wave length and intensity sufficient to cleave at least part of the linker of a conjugate as described above thereby releasing the drug from the drug conjugate.

Another aspect of the present invention refers a method for regulating the blood sugar of a patient comprising the steps of
Measuring the blood sugar of a patient;
Comparing the result with a standard value; in one preferred embodiment, this can be performed manually, or, more preferably, digitally performed by a computer (controller device);
Calculating the required amount of drug, preferably insulin, to regulate the blood sugar level of a patient; this can be in one preferred embodiment performed manually, or, more preferred, digitally performed by a computer;
Emitting UV light by a UV-light source device which is controlled (directly or via a regulator device) by a controller (for example either a remote control which can be handled manually, or digitally) in a time frame and intensity which results in setting free the required amount of said drug, preferably insulin, from a depot according to the invention.

One further aspect refers to the use of a conjugate according to the invention or a depot comprising at least one conjugate according to the invention for emergency care.

Many disease conditions are life threatening and require quick action. In particular, biomolecule drugs (e.g. peptides, proteins) are cumbersome to administer in these situations, as they require usually quick injection of a drug solution, sometimes by a layman. An example is glucagon administration for treatment of severe hypoglycemia for treatment of Type 1 Diabetes. Also small molecule drugs sometimes have to be injected in order to ensure rapid onset of action (e.g. epinephrine and ant-histamines for treatment of anaphylactic shock, benzodiazepines for treatment of epileptic seizures, atropine for treatment of intoxication by acetylcholine esterase inhibitors).

Another aspect refers to the use of a conjugate according to the invention or a depot comprising at least one conjugate according to the invention for tumor surveillance in difficult to reach areas e.g. the brain. As tumors tend to return in glioblastoma after resection, a smart depot could be implanted to easily liberate drug molecules at the site of reoccurrence without the need of further brain surgery as long as the depot can release a drug in the required amount.

Another aspect refers to the use of a conjugate according to the invention or a depot comprising at least one conjugate according to the invention for needle-free application of biological drugs like antibody drugs or RNA. Many effective antibodies are available to date e.g. for treatment of immune-mediated and inflammatory diseases (like psoriasis, e.g. secukinumab, Crohn' disease e.g. or multiple sclerosis e.g. ocrelizumab).

Another aspect of the invention refers to the use of a compound of formula (I), (II) or (III) for immobilizing a drug which comprises a carboxylic acid group.

This novel linker was demonstrated to work successfully on chemical structures of various features, for instance peptides, small molecules containing carboxylic acid and phenols. It is believed that this new invention could be used for virtually every drug that contains a reactive group with one heteroatom available to be bonded to the alfa position of a diazirine linker, preferably an asymmetric diazirine linker wherein the C-atom of one alpha position comprises at least one hydrogen atom (e.g. Rc and/or Rd is H) and the C-atom of the other alpha position of the diazirine group is not bond to a hydrogen (e.g. Ra and Rb are not H).

### EXPERIMENTAL SECTION

### General Information:

Chemicals and solvents were purchased from commercial suppliers and were used without further purification unless otherwise noted. Petrol ether was distilled prior to use. For thin layer chromatography (TLC) aluminium backed silica gel 60 F254 (from Merck) was used. The visualization occurred by UV fluorescence (254 nm) or by staining with KMnO₄ (30% in H₂O). Flash column chromatography was performed using the Reveleris® PREP purification system from BUCHI using silica gel (Eco Flex, particle size 40 μm irregular) or C18-reversed phase silica gel (Eco Flex, particle size 40 μm spherical) as specified for each protocol (the eluent is given in volume ratios (v/v)). NMR spectra were recorded on a Bruker Ultrashield 400 MHz Avance-I at room temperature. Chemical shifts δ are given in ppm relative to residual protonated solvent peaks (CHCl3: δH=7.26 ppm, δC=77.2 ppm; DMSO: δH=2.50 ppm; δC=39.5 ppm; CH3OH: δH=3.31 ppm; δC=49.0 ppm). Coupling constants (J) are given in Hertz. The following descriptions are used for 1H spectra: s (singlet), bs (broad singlet), d (doublet), t (triplet), q (quartet), m (multiplet) or combinations of these acronyms. Electrospray mass spectra were recorded using either a Waters QTOF-Premier (Waters Aquity Ultra Performance, ESI) or a LCT Premier (Waters) with the samples solubilized in methanol. The ionization modes, the calculated mass and found mass are given. For the experiments using coumarin derivatives, UV-vis spectra and fluorescence spectrum were obtained with the Cytation 5 (Cell Imaging Multi-Mode Reader) microplate reader. UV-vis absorption spectra were recorded using the UV-vis spectrophotometer Nanodrop-200C, Nanodrop, USA. Fourier transform infrared (FTIR) spectra were performed with a Tensor 27 FTIR spectrophotometer. Spectra of the irradiating sources were determined using an Edinburgh Instruments (UK) LP-900 laser kinetic spectrometer.

The proof of principle was performed by observing the release of a test molecule by proton nuclear magnetic resonance (1H and 13C-NMR) and/or liquid chromatography and/or TLC and/or by emission spectroscopy for luminescent compounds.

For molecules with molecular weight greater than 1000 g/mol, chromatography was performed on a Waters Acquity UPLC system, equipped with a binary solvent manager, sample manager and column heater. Analysis were performed on an Acquity UPLCO Protein BEH C4 (1.7 μm, 2.1×50 mm, 1 pkg) column kept at 40° C. The mobile phase consisted of 0.1% formic acid in water (A) and in acetonitrile (B).

A gradient elution was performed at 0.6 mL/min by starting with 95% of eluent A and 5% eluent B, then applying a linear gradient to 00% of eluent A and 100% eluent B in 4.15 min., then applying a linear gradient to 95% of eluent A and 05% eluent B in 0.5 min. The total run time, including re-equilibration, was 05 min and the injection volume was 10 μL in positive ionization modes, using the partial-loop fill mode. Mass spectrometry was performed on a Waters Synapt G2-S mass instrument (Waters MS Technologies U.K.) equipped with an electrospray ion (ESI) source operated in positive (ESI+) polarity. All mass spectra data were collected in centroid mode using the MS mode of operation.

For molecules with molecular weight lower than 1000 g/mol, chromatography was performed on a Waters Acquity H-Class UPLC system, equipped with a binary solvent manager, sample manager and column heater. Analysis were performed on a Acquity UPLCO BEH C18 (1.7 μm, 2.1×50 mm) column kept at 40° C. The mobile phase consisted of 0.1% formic acid in water (A) and in acetonitrile (B). A gradient elution was performed at 0.8 mL/min by starting with 95% of eluent A and 5% eluent B for 1.0 min, then applying a linear gradient to 05% of eluent A and 95% eluent B in 1.25 min., then applying a linear gradient to 95% of eluent A and 05% eluent B in 0.5 min. The total run time, including re-equilibration, was 03 min and the injection volume was 5 μL in positive ionization modes, using Waters Acquity Qda detector and with Acquity UPLC PDA detector (ACQUITY UPLC Photodiode Array (PDA) Detector).

The compound GLP-1-alkyne was prepared by solid-phase peptide synthesis method using in situ neutralization for fluorenylmethoxycarbonyl (Fmoc)-based chemistry similar to the method described by Tschöp et al. (Nat. Med. 2012, 18 (12), 1847-1856). Here, it was used L-C-propargylglycine (CAS number 23235-01-0) instead of Lysin (K) at position 39.

The compound Glucagon-alkyne was prepared by solid-phase peptide synthesis method using in situ neutralization for fluorenylmethoxycarbonyl (Fmoc)-based chemistry similar to the method described by Tschöp et al. (Nat. Med. 2012, 18 (12), 1847-1856). Here, the natural sequence was prolonged with L-C-propargylglycine (CAS number 23235-01-0) at position 30.

The compound Insulin-azide was prepared similar to the method described by Lickert et al. (Nature 2021, 590, 326-331). Here, 2,5-dioxopyrrolidin-1-yl 4-azidobutanoate instead of hept-6-ynoic acid NHS ester was used.

### Example 1: Synthesis of Linker Molecules

### Example 1a: (3,3-dihydroxymethyl)-diazirine

Scheme 2 shows the route of synthesis of a linker (linker A in Scheme 2) according to the invention.

To 250 ml NH₃ at −78° C. dihydroxyacetone (5.0 g) previously dissolved in methanol (140 ml) was added. After 3 h stirring at −60° C., hydroxyamine O-sulfonic acid (1.0 eq) dissolved in methanol (60 ml) was added dropwise. The reaction mixture was brought to room temperature overnight and remaining ammonia was removed by bubbling argon through the mixture.

Without further purification, at 0° C., the intermediate diaziridine was oxidized to diazirine by adding a solution of triethylamine (1.5 eq., 12 ml) in methanol (50 ml). After that, solid iodine (9.57 g) was slowly added to the mixture and the resulting mixture was stirred for an additional 2 h at room temperature, after which the methanol was evaporated under reduced pressure. The mixture was extracted with ethyl acetate (100.0 ml), dried over Na₂SO₄ and purified by flash column chromatography using 80 g silica gel column as stationary phase and a mixture of petrol ether (PE) and ethyl acetate (EtOAc) (Flow 70 mL per minute; mixture gradient: from pure PE to pure EtOAc in 40 minutes) as eluent to afford product as colorless oil in 55% isolated yield.

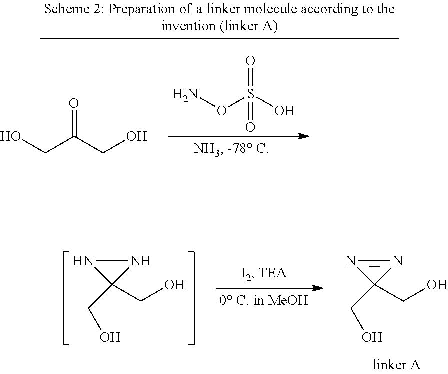

The preparation of this molecule (linker A) was described in Schultz et al. Biochemistry 2018, 57, 31, 4747-4752. Linker A was obtained in 56% yield (transparent oil).

R_{f}=0.50 (EtOAc [KMnO4]).

¹H NMR (400 MHz, MeOD) δ 4.86 (s, 2H), 3.48 (s, 4H).

¹³C NMR (101 MHz, MeOD) δ 61.80.

### Example 1b: (3-(hydroxymethyl)-3H-diazirin-3-yl)methyl 6-azidohexanoate

Under argon, linker A (1.72 mmol), 4-(dimethylamino)pyridine (DMAP, 3.2 mmol) and a mixture of DCM/DMF (1:1, v:v) (3 ml) were stirred for 2 minutes. After that, 6-azidohexanoic acid (0.92 eq) was added at once. The resulting mixture was stirred at room temperature for 5 minutes and ethylcarbodiimide hydrochloride EDC-Cl (3.0 mmol) was added. The resulting mixture was stirred at room temperature overnight, the solvent was removed under reduced pressure and the final product was purified by flash column chromatography using 40 g silica gel column as stationary phase and a mixture of petrol ether (PE) and ethyl acetate (EtOAc) (Flow 40 mL per minute, mixture gradient: from pure PE to pure EtOAc in 30 minutes) as eluent to afford product as colorless oil in 44% isolated yield.

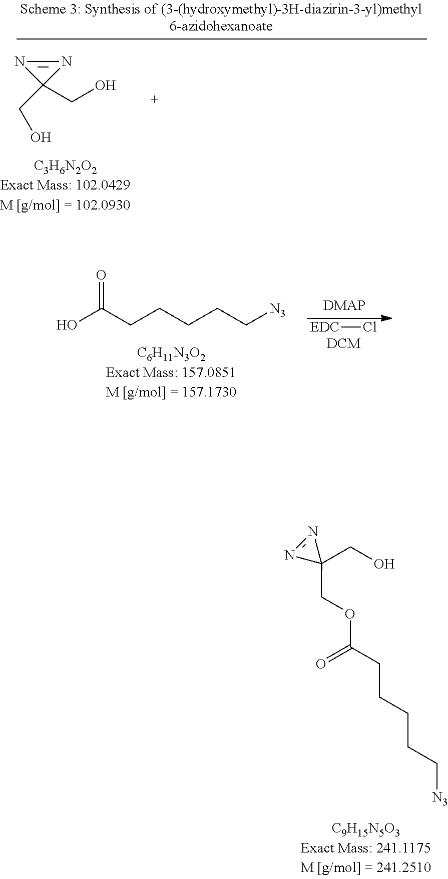

R_{f}=0.47 (PE/EtOAc (3:1) [KMnO₄].

¹H NMR (400 MHz, CDCl₃) δ 4.02 (s, 2H), 3.50 (s, 2H), 3.29 (t, J=6.8 Hz, 2H), 2.38 (t, J=7.4 Hz, 2H), 1.74-1.58 (m, 4H), 1.49-1.38 (m, 2H).

¹³C NMR (101 MHz, CDCl₃) b 173.67, 63.93, 63.04, 51.34, 33.84, 28.66, 26.29, 24.49.

### Example 1c: (3-(hydroxymethyl)-3H-diazirin-3-yl)methyl pent-4-ynoate

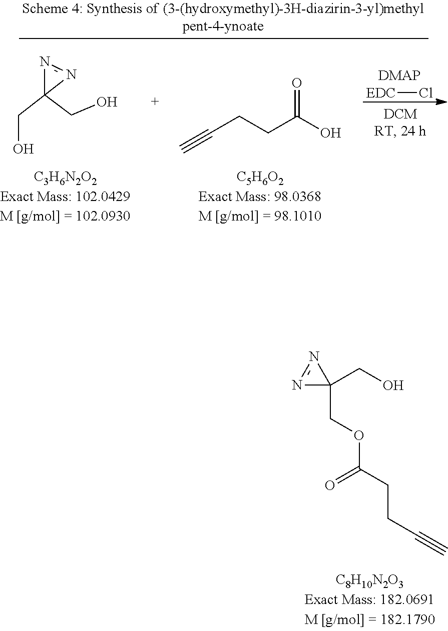

Reaction was performed according to Example 1b using 1.96 mmol of linker A and 0.92 eq. of 4-pentynoic acid. After purification by flash column chromatography using 40 g silica gel column as stationary phase and a mixture of petrol ether (PE) and ethyl acetate (EtOAc) (Flow 40 mL per minute; mixture gradient: from pure PE to pure EtOAc in 30 minutes) as eluent to afford product as colorless oil in 41% isolated yield.

R_{f}=0.73 (EtOAc [KMnO4]).

¹H NMR (400 MHz, CDCl₃) δ 4.04 (s, 2H), 3.52 (s, 2H), 2.67-2.57 (m, 2H), 2.57-2.50 (m, 2H), 2.01 (t, J=2.6 Hz, 1H).

¹³C NMR (101 MHz, CDCl₃) b 171.94, 82.23, 69.48, 64.36, 62.79, 33.18, 14.46.

HRMS (ESI) m/z: [CH₁₀N₂O₃+Na]⁺ calcd.: 205.0589; found: 205.0585.

### Example 1d: (3H-diazirine-3,3-diyl)bis(methylene) bis(pent-4-ynoate)

Reaction was performed exactly according to Example 1c and di-acylated compound can be considered the by-product of example 1c. After purification by flash column chromatography using 40 g silica gel column as stationary phase and a mixture of petrol ether (PE) and ethyl acetate (EtOAc) (Flow 70 mL per minute; mixture gradient: from pure PE to pure EtOAc in 40 minutes) as eluent to afford product as colorless oil in 30% isolated yield.

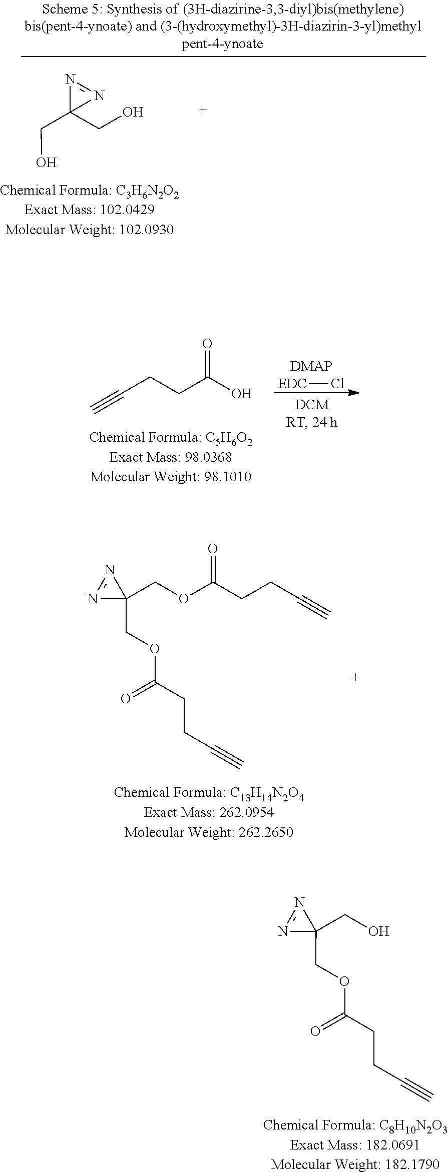

R_{f}=0.80 (EtOAc [KMnO4]).

¹H NMR (400 MHz, CDCl₃) δ 4.02 (s, 4H), 2.64-2.56 (m, 4H), 2.56-2.47 (m, 4H), 2.00 (t, J=2.6 Hz, 2H).

¹³C NMR (101 MHz, CDCl₃) b 171.31, 82.19, 69.47, 64.32, 33.12, 14.44.

### Example 1e: (3-(chloromethyl)-3H-diazirin-3-yl)methyl pent-4-ynoate

Under argon, the corresponding alcohol ((3-(hydroxymethyl)-3H-diazirin-3-yl)methyl pent-4-ynoate, example 1c) (0.29 mmol) was diluted in DCM (2.0 ml) at room temperature followed by the addition of triethylamine (8.0 eq). The mixture was stirred for 3 minutes, cooled down with an ice bath, and methanesulfonylchloride (5.0 eq) was added. The ice bath was removed and the mixture was stirred overnight. before being quenched with a saturated NH₄Cl_{(aq)} solution (5.0 ml). After phases separation, the aqueous phase was extracted with DCM (2×4.0 ml). The combined organic layers were dried over Na₂SO₄ and the residue was purified by flash column chromatography using 4 g silica gel column as stationary phase and a mixture of petrol ether (PE) and ethyl acetate (EtOAc) (Flow 15 mL per minute; mixture gradient: from pure PE to pure EtOAc in 20 minutes) as eluent to afford product as colorless oil in 60% isolated yield.

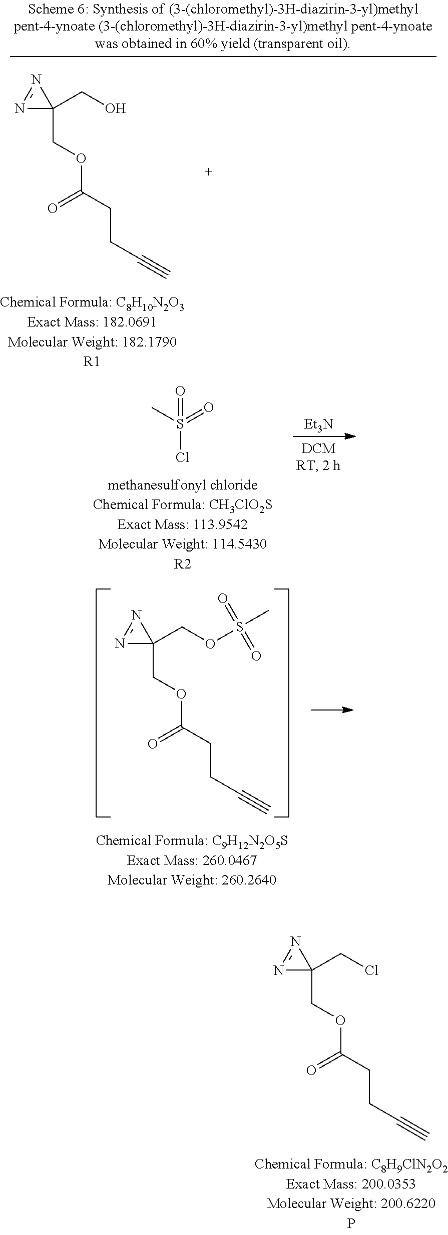

R_{f}=0.55 (PE/EtOAc (3:1) [KMnO4].

¹H NMR (400 MHz, CDCl₃) δ 4.07 (s, 2H), 3.42 (s, 2H), 2.64-2.60 (m, 2H), 2.56-2.51 (m, 2H), 2.00 (t, J=2.6 Hz, 1H).

¹³C NMR (101 MHz, CDCl₃) b 171.29, 82.16, 69.49, 63.82, 45.83, 33.15, 14.46.

### Example 1f: (3-(iodomethyl)-3H-diazirin-3-yl)methyl pent-4-ynoate

Under argon, 0.13 mmol (3-(chloromethyl)-3H-diazirin-3-yl)methyl pent-4-ynoate was diluted in acetone (2.0 ml) and the resulting mixture was stirred under argon for 1 minute, followed by the addition of solid KI (3.0 eq.). The mixture was stirred for 3 hrs. at 40° C., filtrated (using Chromafil Xtra, 13 mm, 0.2 μm), the remaining solid was washed with dried acetone (1.0 ml). The combined crude in acetone was concentrated under reduced pressure and the corresponding iodide was obtained in quantitative yield according to 1H-NMR analysis. PS: product was used in the next step without further purification.

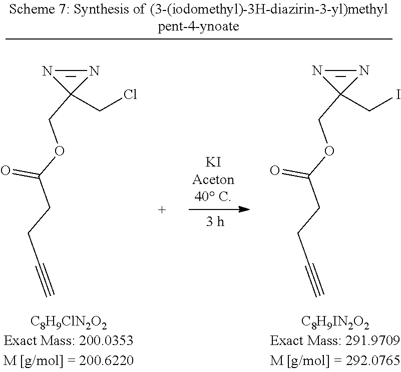

R_{f}=0.56 (PE/EtOAc (3:1) [UV, KMnO4].

¹H NMR (400 MHz, CDCl₃) δ 4.05 (s, 2H), 3.01 (s, 2H), 2.63 (dd, J=10.7, 4.1 Hz, 2H), 2.58-2.50 (m, 2H), 2.00 (t, J=2.6 Hz, 1H).

¹³C NMR (101 MHz, CDCl₃) b 171.25, 82.18, 69.51, 64.16, 33.17, 14.46, 6.37.

### Example 2: Synthesis of Conjugates, Intermediates and Intermediate Conjugates

Various conjugates and intermediate conjugates (only one reactive group of a linker molecule is reacted with a corresponding group of a compound, the second reactive group of the linker molecule is not yet substituted) were prepared to proof the general concept of forming linkers according to the invention.

### Example 2a: Wherein Y and W of a Conjugate Represent *—O—C(O)—

Under argon and at room temperature, linker A according to Example 1a (amount specified on each reaction below) and 4-(dimethylamino)pyridine (DMAP, 3.2 mmol) were mixed followed by dilution with dichlormethane/dimethylformamide (DCM/DMF (1:1, v:v), 3 ml) and addition of the desired carboxylic acid (1.0-3.0 mmol, check individual protocols). After 5 minutes, EDC-Cl (3.0 mmol) was added. The resulting mixture was stirred overnight, the solvent removed under reduced pressure and the respective final product was purified by column chromatography (normal or reverse phase).

Example a) refers to a drug conjugate according to the invention with naproxen, a pain killer. Examples b) and c) were prepared to provide the general proof of concept for the drug conjugates according to the invention (Scheme 8).

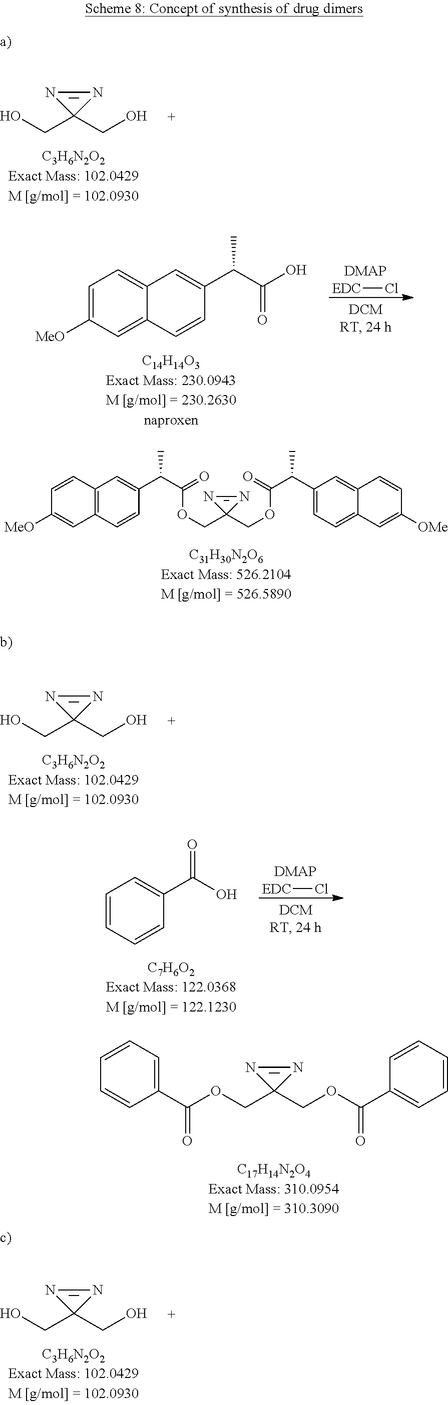

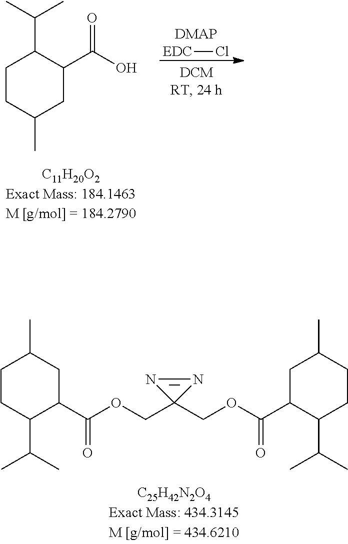

### a) (2S,2′S)-bis(2-(6-methoxynaphthalen-2-yl)propanoate)

Reaction performed according to the general procedure described above using 0.49 mmol of linker A and 2.2 eq. of Naproxen. After purification by flash column chromatography using 12 g silica gel column as stationary phase and a mixture of petrol ether (PE) and ethyl acetate (EtOAc) (Flow 25 mL per minute; mixture gradient: from pure PE to pure EtOAc in 30 minutes) as eluent the product was obtained as white solid in 60% isolated yield.

R_{f}=0.78 (EtOAc [UV, KMnO4].

¹H NMR (400 MHz, CDCl₃) b 7.68 (dd, J=8.6, 3.6 Hz, 4H), 7.62 (s, 2H), 7.34 (dd, J=8.5, 1.6 Hz, 2H), 7.15-7.07 (m, 4H), 3.93-3.87 (m, 8H), 3.87-3.78 (m, 4H), 1.54 (d, J=7.2 Hz, 6H).

¹³C NMR (101 MHz, CDCl₃) b 174.18, 157.85, 135.13, 133.89, 129.42, 129.02, 127.38, 126.21, 126.15, 119.22, 105.72, 64.22, 55.45, 45.27, 18.57.

HRMS (ESI) m/z: [C₃₁H₃₀N₂O₆+Na]⁺ calcd.: 549.2002; found: 549.2000.

### b) (3H-diazirine-3,3-diyl)bis(methylene) dibenzoate

Reaction performed according to general procedure described above using 0.98 mmol linker A and 2.2 eq. benzoic acid. After purification by flash column chromatography using 4 g silica gel column as stationary phase and a mixture of petrol ether (PE) and ethyl acetate (EtOAc) (flow 15 mL per minute; using a gradient: from pure PE to pure EtOAc in 20 minutes) as eluent the product was obtained as transparent oil in 78% isolated yield.

R_{f}=0.43 (PE/EtOAc (5:1) [UV, KMnO4].

¹H NMR (400 MHz, CDCl₃) δ 8.06-8.01 (m, 4H), 7.58 (t, J=7.4 Hz, 2H), 7.43 (t, J=7.7 Hz, 4H), 4.32 (s, 4H).

¹³C NMR (101 MHz, CDCl₃) b 166.11, 133.57, 129.94, 129.29, 128.61, 64.87.

HRMS (ESI) m/z: [C₁₇H₁₄N₂O₄+Na]⁺ calcd.: 333.0851; found: 333.0836.

### c) (3-((((1R,2S,5R)-2-isopropyl-5-methylcyclohexane-1-carbonyl)oxy)methyl)-3H-diazirin-3-yl)methyl(1S,2S,5R)-2-isopropyl-5-methylcyclohexane-1-carboxylate

Reaction performed according to general procedure described above using 0.49 mmol of linker A and 2.2 eq. of 2-Isopropyl-5-methylcyclohexanecarboxylic acid. After purification by flash column chromatography using 4 g silica gel column as stationary phase and a mixture of petrol ether (PE) and ethyl acetate (EtOAc) (Flow 15 mL per minute; mixture gradient: from pure PE to pure EtOAc in 20 minutes) as eluent the product was obtained as transparent oil in 21% isolated yield. R_{f}=0.69 (PE/EtOAc (5:1) [KMnO4].

¹H NMR (400 MHz, CDCl₃) δ 3.98 (s, 4H), 2.34 (td, J=11.7, 3.5 Hz, 2H), 1.85 (dd, J=12.6, 2.1 Hz, 2H), 1.77-1.46 (m, 8H), 1.35 (ddd, J=14.5, 6.7, 3.5 Hz, 2H), 1.18 (q, J=12.2 Hz, 2H), 1.09-0.93 (m, 4H), 0.91 (dd, J=6.7, 2.6 Hz, 12H), 0.80 (d, J=6.9 Hz, 6H).

¹³C NMR (101 MHz, CDCl₃) b 175.77, 63.57, 47.67, 44.61, 38.93, 34.61, 32.20, 29.53, 24.01, 22.39, 21.34, 16.11.

### Example 2b: Wherein Y and/or W of an Intermediate Conjugate Represent *—O—

### (3-(((2-oxo-2H-chromen-7-yl)oxy)methyl)-3H-diazirin-3-yl)methyl-6-azidohexanoate

The coumarin-linker conjugate proofs the general concept of attaching a molecule with a hydroxy group to a linker molecule. The azide function of the linker molecule can then be used for a click chemistry reaction to attach the conjugate to a polymer. Under argon, 0.21 mmol (3-(hydroxymethyl)-3H-diazirin-3-yl)methyl 6-azidohexanoate was diluted in DCM (0.5 ml) at room temperature, followed by the addition of triethylamine (3.2 eq). The mixture was stirred for 2 minutes and cooled down with an ice bath. Methanesulfonyl chloride (3.0 eq.) was slowly added. The ice-bath was removed and the reaction mixture (solution I) was stirred under room temperature for 2 hrs.

To 7-hydroxy-coumarin (2.0 eq) in DMF (0.5 ml), K₂CO₃ (7.0 eq) and KI (0.3 eq.) were added (solution II). After stirring solution II for 10 minutes, solution I was added to solution II and the resulting mixture was stirred at room temperature for 18 hrs. After evaporation of the volatiles under reduced pressure the crude was purified by flash column chromatography using 12 g silica gel column as stationary phase and a mixture of petrol ether (PE) and ethyl acetate (EtOAc) (Flow 25 mL per minute; mixture gradient: from pure PE to pure EtOAc in 45 minutes) as eluent to afford product as yellow-brown oil in 15% isolated yield.

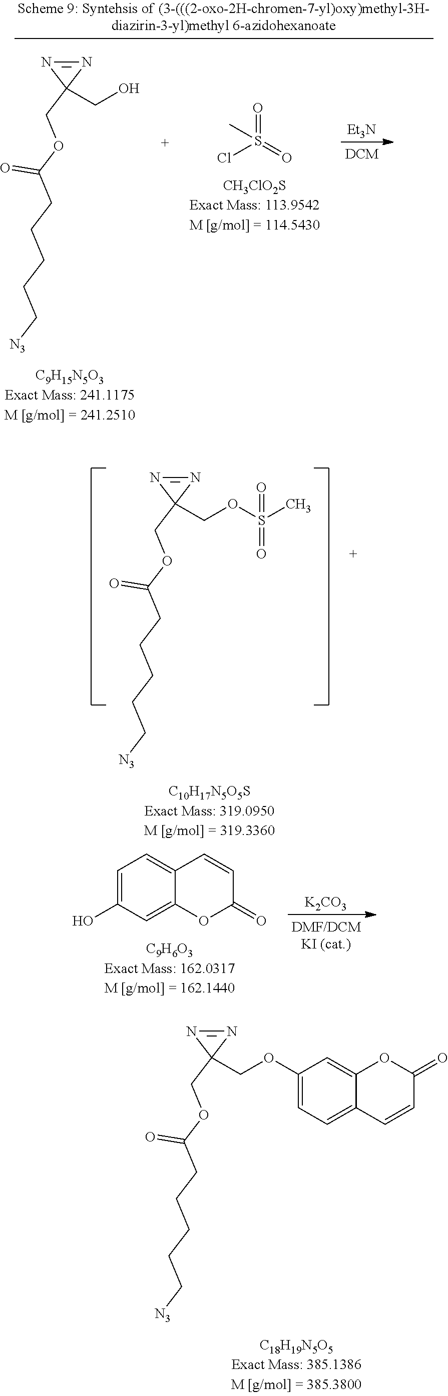

R_{f}=0.55 (PE/EtOAc (1:1) [UV, KMnO4].

¹H NMR (400 MHz, CDCl₃) δ 7.64 (d, J=9.4 Hz, 1H), 7.39 (d, J=8.6 Hz, 1H), 6.82 (dd, J=8.6, 2.5 Hz, 1H), 6.75 (d, J=2.5 Hz, 1H), 6.28 (d, J=9.5 Hz, 1H), 4.08 (s, 2H), 3.97 (s, 2H), 3.27 (t, J=6.8 Hz, 2H), 2.38 (t, J=7.4 Hz, 2H), 1.70-1.59 (m, 4H), 1.42 (t, J=7.3 Hz, 2H).

¹³C NMR (101 MHz, CDCl₃) b 173.02, 161.01, 160.87, 155.85, 143.30, 129.11, 114.02, 113.46, 112.93, 101.92, 68.30, 63.58, 51.32, 33.77, 28.66, 26.53, 24.47.

HRMS (ESI) m/z: [C₁₈H₁₉N₅O₅+Na]⁺ calcd.: 408.1284; found: 408.1273.

### (3-(((2-oxo-2H-chromen-7-yl)oxy)methyl)-3H-diazirin-3-yl)methyl pent-4-ynoate

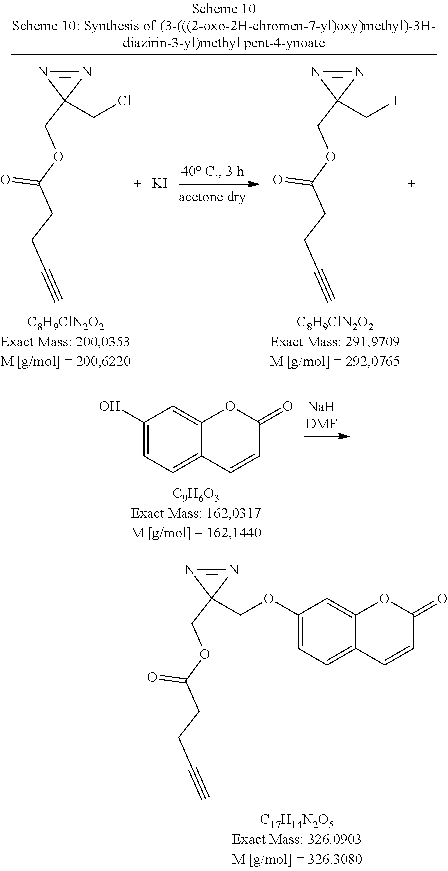

In a round-flask equipped with a stir bar under argon, the corresponding chloride (3-(chloromethyl)-3H-diazirin-3-yl)methyl pent-4-ynoate (0.095 mmol) was diluted in dry acetone (c.a. 1.5 mL) and the resulting mixture was stirred under argon for 1 minute followed by the addition of solid KI (3.0 eq) at once. The mixture was stirred for 3 hours at 40° C., after that, it was filtrated (using Chromafil, Xtra, 13 mm, 0.2 μm) and the remaining solid was washed with dried acetone (1×1.0 mL), and the resulting mixture was further filtrated. The combined crude in acetone was concentrated under reduced pressure and the corresponding iodide (used without further purification) was diluted in dried DMF (c.a 1.0 mL) forming solution I. In parallel, solid sodium hydride (90%) (3.0 eq) was added to a flask, and flushed with argon. DMF (1.0 mL) was added and the mixture was placed in ice-bath and stirred for 2 min under argon atmosphere (solution II). In another flask under argon, the nucleophile (7-hydroxy coumarin) (3.0 eq) was diluted in DMF (1.5 mL) and this solution (solution III) was slowly added over the suspension of sodium hydride (solution II). The resulting mixture of solution II and III was stirred for five minutes under ice-bath and then, the solution of the corresponding organoiodine in DMF (solution I) was slowly added to the resulting mixture containing the nucleophile of the reaction. After 5 minutes, the ice bath was removed and the reaction was stirred under argon atmosphere overnight at room temperature. After that, the volatiles were removed under reduced pressure and the crude was suspended in DCM (5.0 mL) and washed with saturated NH₄Cl_{(aq)} (5.0 mL). The organic phase was separated and the aqueous phase was washed with DCM (2×5.0 mL). The combined organic layers were dried over Na₂SO₄ and, after evaporation of the solvent, the residue was purified by flash column chromatography using 12 g silica gel column as stationary phase and a mixture of petrol ether (PE) and ethyl acetate (EtOAc) (Flow 25 mL per minute; mixture gradient: from pure PE to pure EtOAc in 40 minutes) as eluent to afford product as yellow-brown solid in 43% isolated yield.

R_{f}=0.38 (PE/EtOAc (1:1) [UV, KMnO4].

¹H NMR (400 MHz, CDCl₃) δ 7.64 (d, J=9.4 Hz, 1H), 7.39 (d, J=8.6 Hz, 1H), 6.82 (dd, J=8.6, 2.5 Hz, 1H), 6.75 (d, J=2.5 Hz, 1H), 6.28 (d, J=9.5 Hz, 1H), 4.11 (s, 2H), 3.98 (s, 2H), 2.65-2.59 (m, 2H), 2.55-2.49 (m, 2H), 1.99 (t, J=2.6 Hz, 1H).

¹³C NMR (101 MHz, CDCl₃) b 171.40, 161.10, 160.83, 155.86, 143.30, 129.11, 114.02, 113.46, 112.91, 101.92, 82.17, 69.50, 68.19, 64.03, 33.14, 14.47.

HRMS (ESI) m/z: [C₁₇H₁₄N₂O₅+Na]⁺ calcd.: 349.0800; found: 349.0794.

### c) (3-((5-(2-((tert-butoxycarbonyl)amino)ethyl)-2-hydroxyphenoxy)methyl)-3H-diazirin-3-yl)methyl pent-4-ynoate

The preparation of a conjugate intermediate with a Boc-protected dopamine residue demonstrate the use of the conjugates according to the invention for releasing catecholamines such as adrenaline.

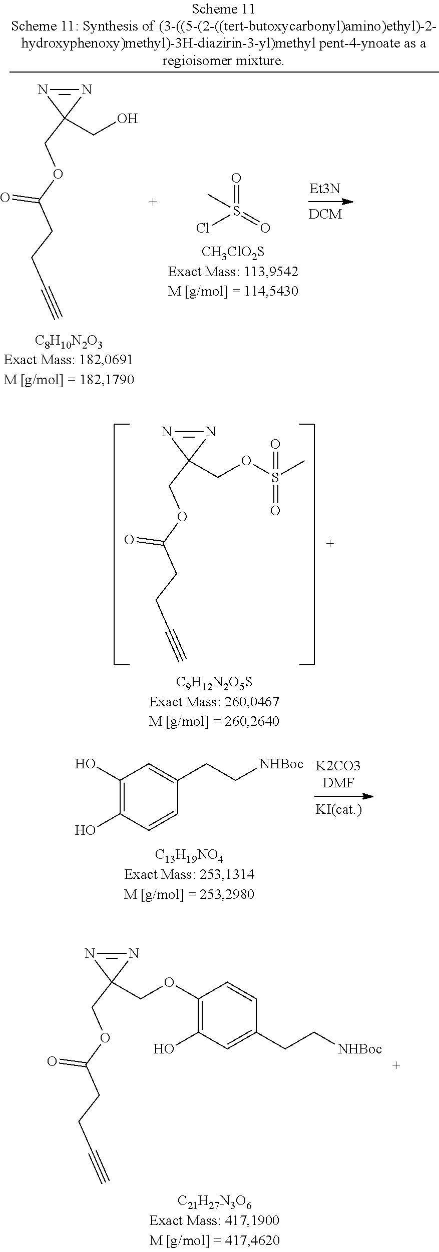

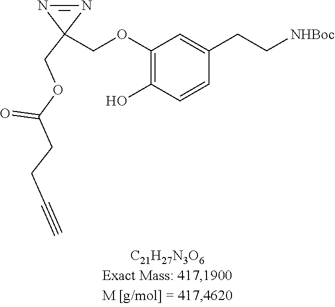 [SMILES: C#CCCC(=O)OCC1(COc2cc(CCNC(=O)OC(C)(C)C)ccc2O)N=N1]

In a round-flask equipped with a stir bar under argon, (3-(hydroxymethyl)-3H-diazirin-3-yl)methyl pent-4-ynoate (0.21 mmol) was diluted in DCM (0.5 mL) at room temperature, followed by the addition of triethylamine (3.2 eq). Next, the mixture was stirred for 2 minutes and cooled down with an ice bath. After this procedure, MsCl (3.0 eq.) was slowly added. The ice-bath was removed and the reaction mixture (solution I) was stirred under room temperature for 2 hrs. In parallel, 2.0 eq of commercially available boc-protected dopamine (tert-butyl (3,4-dihydroxyphenethyl)carbamate) was diluted in DMF (0.5 mL), followed by addition of K₂CO₃ (7.0 eq) and KI (0.3 eq.) (solution II). After stirring solution II for 10 minutes, solution I was added over solution II and the resulting mixture was stirred at room temperature for 18 hrs. After evaporation of the volatiles under reduced pressure the crude was purified by flash column chromatography using 12 g silica gel column as stationary phase and a mixture of petrol ether (PE) and ethyl acetate (EtOAc) (flow 25 mL per minute; using a gradient: from pure PE to pure EtOAc in 40 minutes) as eluent to afford product as a mixture of regioisomers as a transparent oil in 28% isolated yield.

R_{f}=0.74 (PE/EtOAc (1:1) [UV, KMnO₄].

¹H NMR (400 MHz, CDCl₃) δ 6.95-6.61 (m, 28H), 5.74 (d, J=32.2 Hz, 8H), 4.11 (d, J=1.0 Hz, 15H), 3.94 (d, J=6.0 Hz, 16H), 3.34 (d, J=15.4 Hz, 22H), 2.74-2.66 (m, 21H), 2.65-2.58 (m, 18H), 2.53 (ddd, J=8.5, 3.3, 1.1 Hz, 19H), 1.98 (td, J=2.6, 1.8 Hz, 9H), 1.43 (d, J=1.8 Hz, 94H).

HRMS (ESI) m/z: [C₂₁H₂₇N₃O₆+Na]⁺ calcd.: 440.1798; found: 440.1786.

### d) (3-((5-(2-aminoethyl)-2-hydroxyphenoxy)methyl)-3H-diazirin-3-yl)methyl pent-4-ynoate

Deprotecting the Boc-protected conjugate. A round-flask equipped with a stir bar, under argon, was charged with the Boc-protected compound (0.02 mmol) and TFA (1.0 mL). The reaction was stirred for 1 h at room temperature. After that, the volatiles were removed under reduced pressure to give the crude product as a transparent oil in quantitative yield.

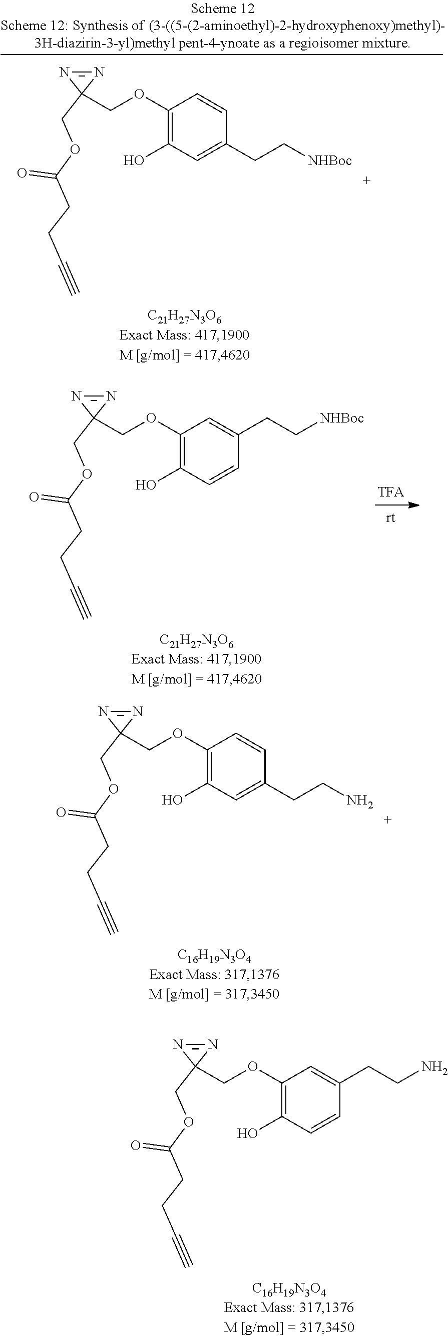 [SMILES: C#CCCC(=O)OCC1(COc2ccc(CCNC(=O)OC(C)(C)C)cc2O)N=N1 ; C#CCCC(=O)OCC1(COc2cc(CCNC(=O)OC(C)(C)C)ccc2O)N=N1 ; "rt" ; "TFA" ; C#CCCC(=O)OCC1(COc2ccc(CCN)cc2O)N=N1 ; C#CCCC(=O)OCC1(COc2cc(CCN)ccc2O)N=N1]

¹H NMR (400 MHz, MeOD) δ 6.86-6.68 (m, 3H), 4.15 (d, J=8.3 Hz, 2H), 4.01 (d, J=4.3 Hz, 2H), 3.35 (s, 2H), 3.16-3.09 (m, 2H), 2.83 (dt, J=16.5, 8.2 Hz, 2H), 2.59-2.53 (m, 2H), 2.49-2.43 (m, 2H), 2.26 (t, J=2.6 Hz, 1H).

### Example 2c: Synthesis of Conjugates Wherein Y and/or W Represent *—O—C(O)—NH—

### (3H-diazirine-3,3-diyl)bis(methylene) bis(benzylcarbamate)

Under argon, carbonyldiimidazol (CDI, 2.0 eq.) was slowly added at room temperature to 0.49 mmol linker A in DCM (2.0 ml). After 1 h, benzyl amine (2.2 eq.) was added to the mixture and the reaction was stirred at room temperature for 3 h. The reaction was quenched with saturated NH₄Cl_{(aq)} (5.0 mL). The aqueous phase was washed DCM (3×5.0 mL). The combined organic layers were dried over Na₂SO₄ and, after evaporation of the solvent, the residue was purified by flash column chromatography using 4 g silica gel column as stationary phase and a mixture of petrol ether (PE) and ethyl acetate (EtOAc) (flow 15 mL per minute; using a gradient: from pure PE to pure EtOAc in 20 minutes) as eluent to afford product as transparent oil in 08% isolated yield.

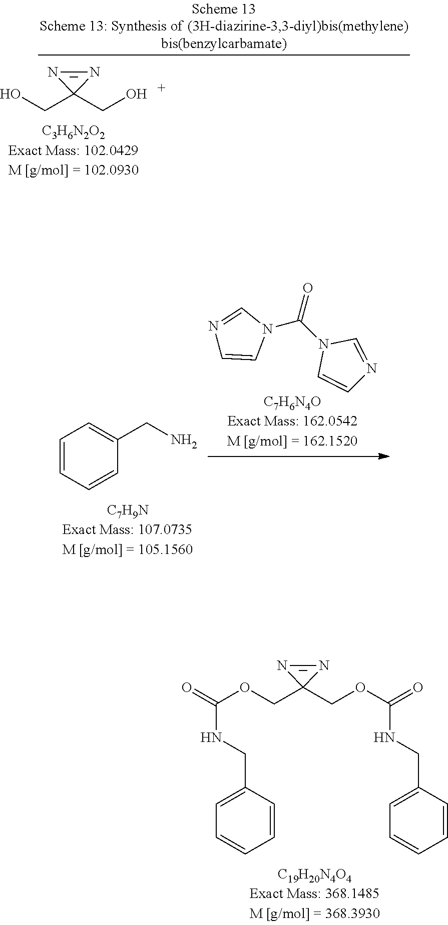 [SMILES: OCC1(CO)N=N1 ; O=C(n1ccnc1)n1ccnc1 ; NCc1ccccc1 ; O=C(NCc1ccccc1)OCC1(COC(=O)NCc2ccccc2)N=N1]

R_{f}=0.20 (PE/EtOAc (5:1) [UV, KMnO₄].

¹H NMR (400 MHz, CDCl₃) δ 7.38-7.24 (m, 10H), 5.06 (s, 2H), 4.36 (d, J=5.8 Hz, 4H), 4.02 (s, 4H).

¹³C NMR (101 MHz, CDCl₃) b 155.73, 138.17, 128.89, 127.81, 127.69, 64.58, 45.36.

### Example 2d: Synthesis of Conjugates Wherein Y and/or W Represent *—N(C(O)—)—

Reaction performed according to general procedure Example 2b, Scheme 10 using 0.20 mmol of the corresponding chloride (3-(chloromethyl)-3H-diazirin-3-yl)methyl pent-4-ynoate and fluoruracil (3.0 eq) as the nucleophile. After purification by flash column chromatography using 4 g silica gel column as stationary phase and a mixture of dichloromethane (DCM) and methanol (MeOH) (flow 15 mL per minute, using a gradient: from pure DCM to DCM/MeOH (9:1) in 30 minutes) as eluent the product was afforded as regioisomer mixture in 50% isolated yield as yellow oil.

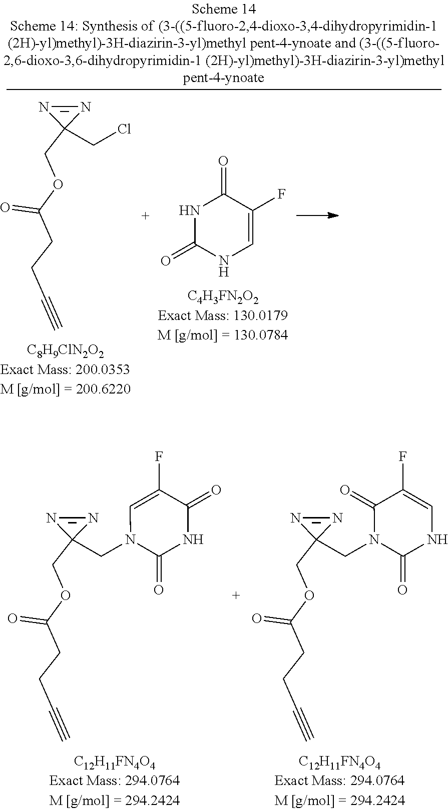 [SMILES: C#CCCC(=O)OCC1(CCl)N=N1 ; O=c1[nH]cc(F)c(=O)[nH]1 ; C#CCCC(=O)OCC1(Cn2cc(F)c(=O)[nH]c2=O)N=N1 ; C#CCCC(=O)OCC1(Cn2c(=O)[nH]cc(F)c2=O)N=N1]

R_{f}=0.75 and 0.63 (DCM/MeOH (9:1) [UV, KMnO4].

¹H NMR (400 MHz, CDCl₃) δ 8.79-8.80 (m, 1H), 7.28 (dd, J=4.7, 1.8 Hz, 1H), 7.15 (dd, J=5.0, 3.1 Hz, 1H), 4.08 (dd, J=7.3, 2.4 Hz, 4H), 4.03 (d, J=0.9 Hz, 2H), 3.85 (d, J=5.6 Hz, 4H), 2.66-2.58 (m, 2H), 2.58-2.49 (m, 4H), 2.04-1.99 (m, 2H).

HRMS (ESI) m/z: [C₁₂H₁₁F₁N₄O₄+Na]⁺ calcd.: 317.0662; found: 317.0662.

### Example 3: Synthesis of a Conjugate Between 7-Hydroxycoumarin and Agarose-Azide

Here, it is described a general strategy to use this diazirine linkers by covalently bind desired molecules (e.g. drugs, fluorophores etc.) to micro-particle surfaces. The desired molecule is attached to the micro-particles by two key steps: 1) attachment of the desired molecule to the diazirine linker containing “clickable motifs” (terminal alkyne or azide) and 2) attachment of this new molecule-linker conjugate to micro particles using click chemistry. 7-Hydroxycoumarin, a fluorophore, was attached to a linker molecule (containing a terminal alkyne (conjugate A, Scheme 15)) according to the protocol as described previously on Scheme 10). The next step was based on the click reaction between conjugate A and the micro-particle surface which in this example is azide agarose (obtained commercially from Jena Bioscience, cat. N. CLK-1038-2).

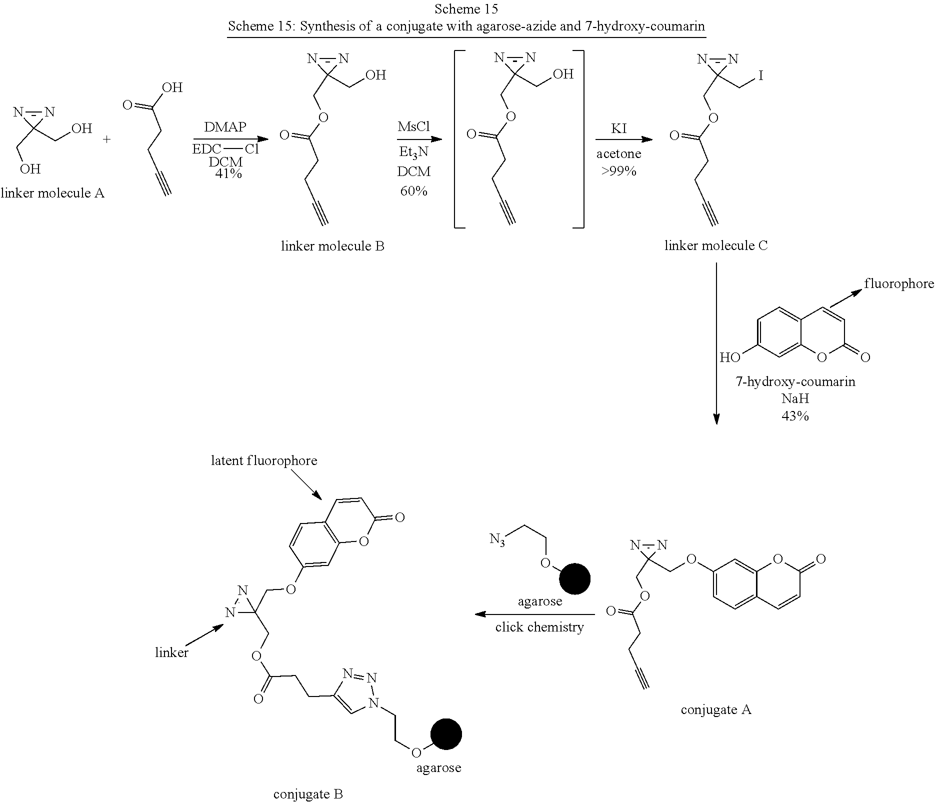

In a 10 mL glass syringe containing a filter at the bottom, a suspension of the commercially available azide agarose (size: 50-150 μm) from Jena Bioscience Cat. N. CLK-1038-2 (0.6 mL, 10-20 μmol/mL) was added. The agarose was washed with dimethylformamide (DMF) (2×1.0 mL). After that, the agarose was suspended in DMF (1.0 mL) followed by addition of a solution of the conjugate A in DMF (4.0 mg in 0.5 mL). After that, solid THTPA (Tris (3-hydroxypropyltriazolylmethyl)amin) (60 eq.) was added to the syringe followed by the addition of CuSO₄ (20.0 eq.) in ultrapure water (0.3 mL). Next, sodium ascorbate (50.0 eq) was diluted in 0.3 mL of ultrapure water and it was added to the syringe. The mixture was shaken on a shaker (100 rpm) at room temperature for 14 h. Next, the supernatant was pushed out and the agarose was washed with DMF (4×1.0 mL). After that, the suspension was suspended in 2.0 mL of a mixture ultrapure water/EtOH (1:1) and stored in the refrigerator. Infrared analysis of the final product shows that the typical IR band of azides were to some extend consumed indicating the formation of the product.

It is important to mention that 7-hydroxycoumarin shows a strong fluorescence at around 450 nm. Nevertheless, when the 7-hydroxy group of the coumarin is transformed into an ether (as for instance conjugate A in Scheme 15), the molecule displays very low or even lack fluorescence. These features make 7-hydroxycoumarin a good candidate to show, by fluorescence experiments, that the diazirine linker can release free 7-hydroxycoumarin.

### Example 4: Breakdown of the Diazirine Linker

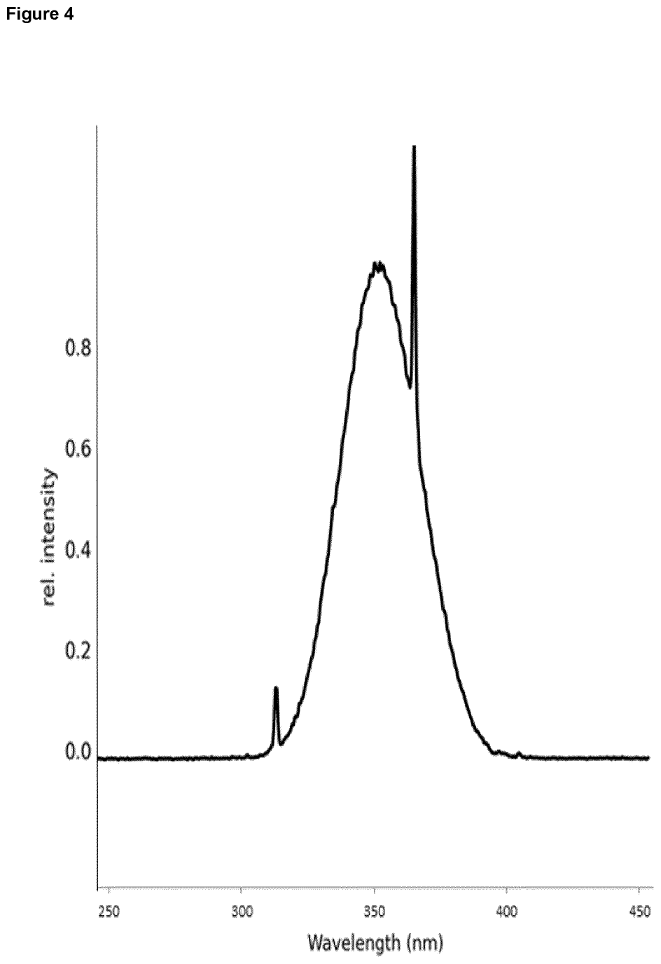

The dimers obtained in Example 2a a) to c) were dissolved in methanol (5 mg per 3 mL of solvent) and placed at room temperature (for the naproxen dimer additionally, a few drops of DCM were added). After that, 100 μL of the resulting solution was separated and protected from light to be used on TLC, LC-MS and/or NMR analysis. Then, irradiation was performed using a TLC lamp (UV-light source had the spectrum as shown in FIG. 4 with a sharp peak at 366 nm and significant emission between 320 nm and 380 nm) for the indicated amount of time (in these examples 20 minutes). Progress of the reaction was followed by TLC, LC-MS and/or NMR using the commercially available naproxen, benzoic acid and menthyl acid as standards.

In all cases, the liberation of the free carboxylic acid (free drug) could be detected (by LC-MS and TLC (PE/EtOAc (5:1) [KMnO₄]))). The commercial drug Naproxen and the commercial acids (benzoic and menthyl acid) were used as standards to demonstrate the liberation of the drug (of a compound being bound to a linker via a reaction of a reactive group of said compound with a reactive group of a linker molecule) upon irradiation with UV-light.

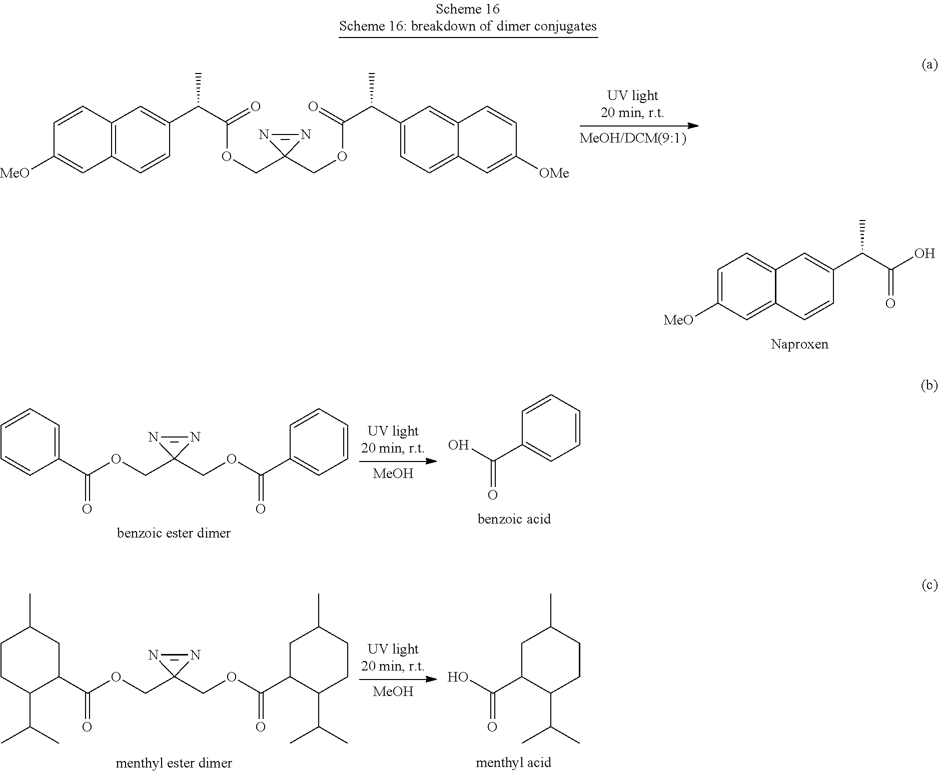

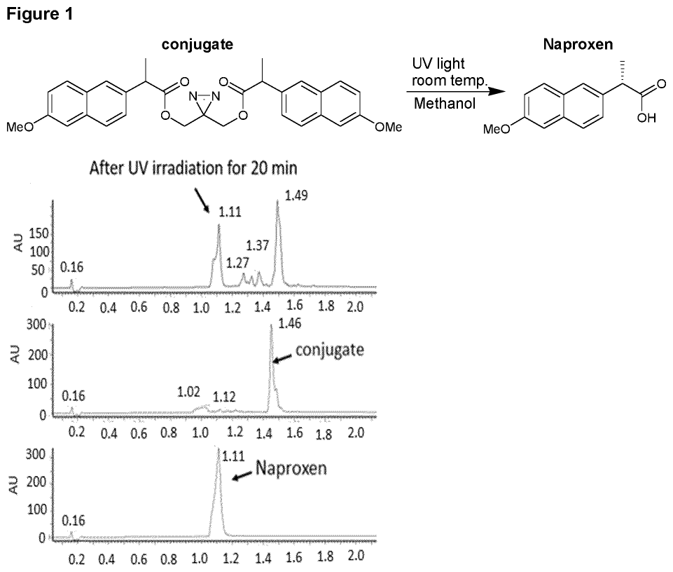

The identification of the release of the native form of Naproxen from naproxen dimer conjugate upon UV-light irradiation was demonstrated by LC-MS. Release of Naproxen upon UV light irradiation measured by liquid chromatography after 20 min UV light irradiation is demonstrated in FIG. 1. The results with benzoic and menthyl acid confirmed the results with Naproxen.

### Example 5: Kinetic Study of the UV Light Release of 7-Hydroxy Coumarin

In a syringe containing a filter at the bottom, the agarose containing the 7-hydroxycoumarin (connected through the diaziridine linker) was washed 3 times with 2.0 mL of a solution of water/EtOH (1:1). Then 3 mL of water/EtOH (1:1) solution were added and the heterogeneous solution was transferred to a 96-well plate (well plate-A) (100 μL per well, a total of 27 wells). After 5 minutes, before irradiation, 3 samples (experiment performed in triplicates) from well plate-A were transferred to a second 96-well plate (well plate-B) which was kept in the dark. Well plate-A was placed under UV light and irradiation was initiated. After each indicated time of irradiation (1 min, 3 min, 6 min, 10 min, 20 min, 60 min, 90 min, and 120 min), 3 samples were transferred form well plate-A to well plate-B. After the end of the irradiation time (120 min), well plate-B was centrifuged (1 minute at 1000 rpm at room temperature) and 60 μL of the supernatant was transferred from well plate-B to a third well plate (well plate-C). In triplicate, 60 μL of standards solutions of 7-hydroxycoumarin in ultrapure water/EtOH (1:1) (concentrations of 0, 3, 6, 10, 20, 40 and 60 μM) were placed on well plate-C. After that, well plate-C was placed in a microplate reader (Cytation 5) and the fluorescence intensity of each well was determined (all wells were exited using 325 nm and emission was recorded at 450 nm) by emission spectroscopy. The final concentration of 7-hydroxycoumarin was obtained after comparison of the fluorescence intensity of each well (average of 3 measurements) with the emission of the standard solutions of 7-hydroxycoumarin.

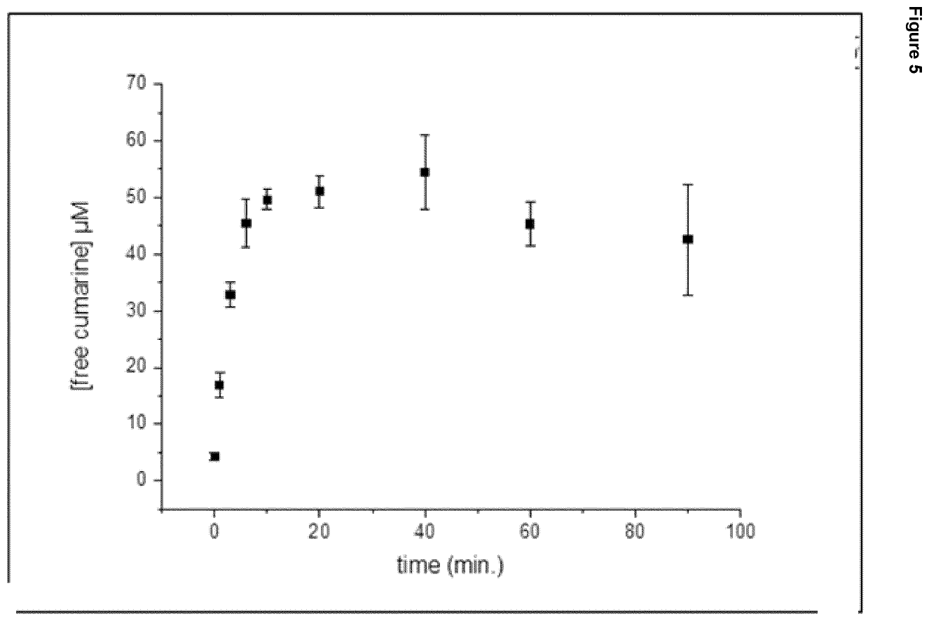

The photo-release of free 7-hydroxy coumarin upon light irradiation was linear from time zero to six minutes (see FIG. 5). FIG. 5 shows the concentration of the released 7-hydroxycoumarin from the linker conjugate B. The releasing constant in this experiment is approximately 6.7 μM/min {c=[6.7 (μM/min)×min]+ 8.2 μM}. The concentration was determined by emission spectroscopy (Abs₍ₘₐₓ₎=325 nm. Em₍ₘₐₓ₎=450 nm) correlated to the irradiation time using UV light.

After 10 minutes, no further increase on the 7-hydroxy coumarin could be detected due to the complete release of the fluorophore from the micro-particle surface. The agarose material that was decanted after centrifugation (well plate-B) should still contain 7-hydroxycoumarin attached to the polymer. To prove that, the remaining linker conjugate B (decanted on well plate-B) was re-suspended in 100 μL water/ethanol (1:1) per well and all wells were further irradiated for 10 minutes. After that, well plate-B was centrifuged (1 minute at 1000 rpm at room temperature) and 60 μL of the supernatant was transferred from well plate-B to a fourth well plate (well plate-D). Well plate-D was placed in a microplate reader (Cytation 5) and the fluorescence intensity of each well was determined (all wells were exited using 325 nm and emission was recorded at 450 nm).

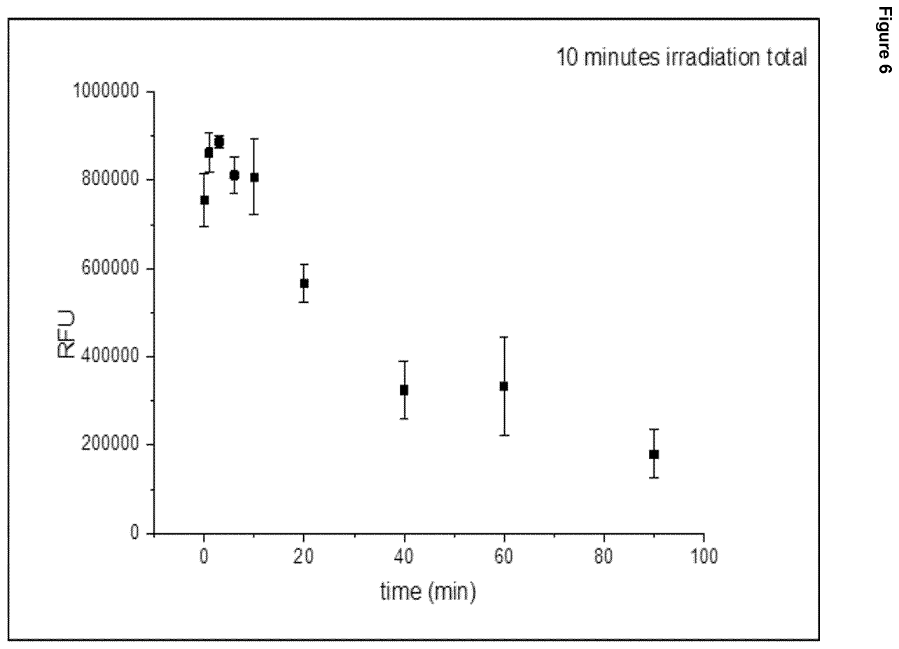

FIG. 6 shows the concentration of the released 7-hydroxy coumarin determined by emission spectroscopy (Abs₍ₘₐₓ₎=325 nm. Em₍ₘₐₓ₎=450 nm) after re-suspending the previously irradiated material in water/ethanol and irradiating with UV light for 10 minutes. It showed that the linker conjugate B can release 7-hydroxy coumarin in batches and the release rate is correlated to the exposure to UV-light. The results on FIG. 6 show that the material can be re-exposed for several times and it will still release the desired drug. Release of 7-hydroxycoumarin and the expected by-product were also determined by LC-MS and TLC (PE/EtOAc (1:1), UV, KMnO₄) (due to the symmetric linker).

An additional experiment was done to show that the release of 7-hydroxy coumarin was only light dependent. Similar to the previous kinetic experiment, a time frame was added in between the light pulses In this experiment, 7-hydroxicoumarin was attached to the linker containing a azide clickable motif (see scheme 9). With this compound in hand, the experiment was simplified and it was not attached to agarose. It was done in a homogenous media.

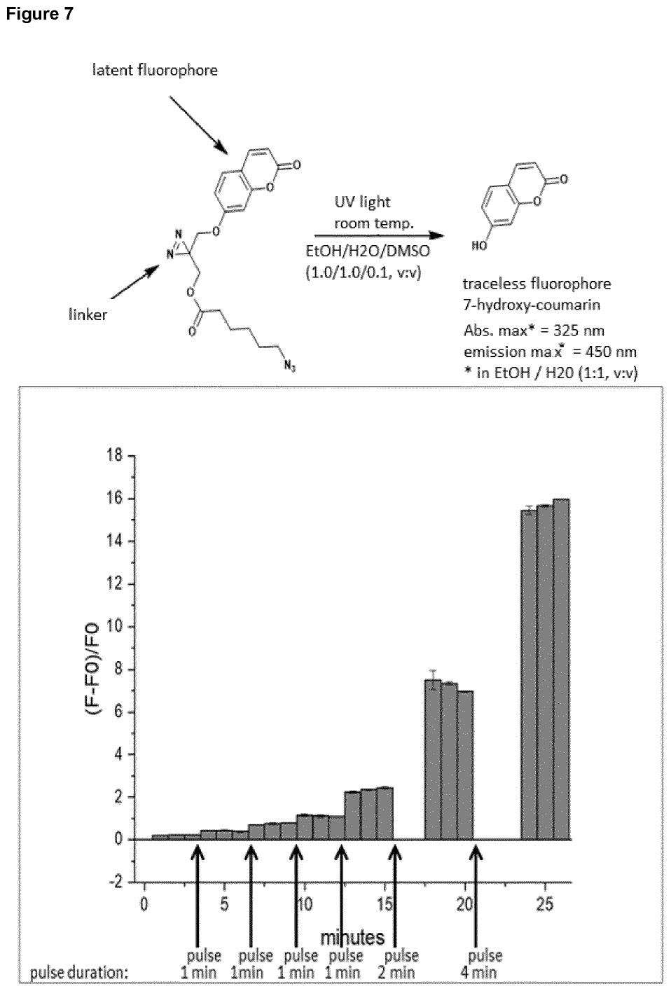

9.0 mg of (3-(((2-oxo-2H-chromen-7-yl)oxy)methyl)-3H-diazirin-3-yl)methyl 6-azidohexanoate was solubilized in 30 mL of ultrapure water/EtOH/DMSO (1.0:1.0:0.1) and the resulting solution was transferred to a 96-well plate (well plate-1) (100 μL per well). After 5 minutes, before irradiation, 2 samples (experiment performed in duplicate) from well plate-1 were transferred to a second 96-well plate (well plate-2) which was kept in the dark. Well plate-1 was placed under UV light and irradiation was initiated. After each pulse of UV light irradiation (pulses duration can be found in FIG. 7), the light source was turned off and 2 samples were transferred from well plate-1 to well plate-2. After each light pulse, well plate-1 was kept for 1 minute in the dark, and, after that, 2 samples were transferred from well plate-1 to well plate-2. After 1 more minute in the dark (2 minutes in total), another 2 samples were transferred from well plate-1 to well plate-2. Two extra 2 minutes in the dark were studied after all pulses of irradiation. After the end of the irradiation, well plate B was placed in a microplate reader (Cytation 5) and the relative concentration of 7-hydroxycoumarin was determined by measuring the fluorescence intensity of each well (average of 2 measurements) (wells were exited using 325 nm and emission was recorded at 450 nm).

As for the result obtained with agarose, efficient release of 7-hydroxycoumarin upon UV light irradiation was observed. In addition, additional release of 7-hydroxycoumarin was observed in the samples that were kept in the dark after the light pulse. This fact indicates that the release is the result of a fast and efficient process which is proportional to the applied light exposure and no additional leaking of payload occurs after ending the pulse (see FIG. 7).

### Example 6: Preparation of Exendin 40, a GLP-1 Derivative

The Glucagon-like peptide 1 (GLP-1) derivative -alkyne was prepared by Fmoc-based solid-phase peptide synthesis method similar to the method described by Tschöp et al. (Nat. Med. 2012, 18 (12), 1847-1856). Here, the natural sequence was prolonged with L-C-propargylglycine (CAS number 23235-01-0) at the C-terminal end.

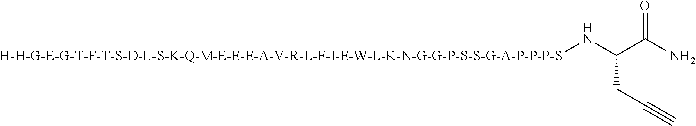

Amino acid sequence shown in SEQ ID NO: 1.

HRMS (ESI): m/z for C₁₈₉H₂₉₂N₅₁O₆₁S [M+5H]⁵⁺: calcd. 856.8207, found: 856.8207.

### Example 7: Synthesis of GLP-1 Conjugate (Drug-Linker-Polymer Conjugate of Formula (A1)) with Exendin-40 (a GLP-1 Derivative)

An alkyne derivative of GLP-1 (Exendin-40) was used as starting material and it was attached to the micro-particle of agarose. For this approach, the general strategy was based on 3 key steps: 1) the acylation of the diazirin linker ((3-(hydroxymethyl)-3H-diazirin-3-yl)methyl 6-azidohexanoate) with succinic anhydride, followed by 2) the incorporation of the linker on the surface of micro-particles by EDC-Cl coupling and 3) the attachment of Exendin-40 by click chemistry.

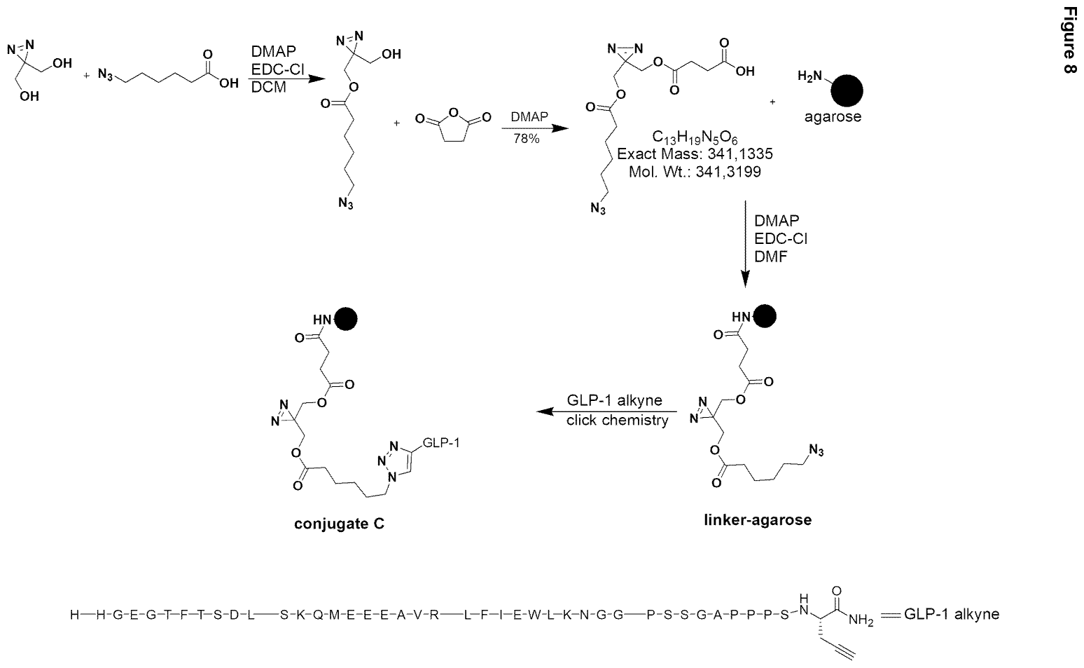

The scheme of this reaction of a GLP-1 conjugate according to the invention is provided in FIG. 8.

Step 1) In a round-flask equipped with a stir bar under argon, was added (3-(hydroxymethyl)-3H-diazirin-3-yl)methyl 6-azidohexanoate) (0.52 mmol) and DMAP (1.2 eq) followed by dilution with DCM (3 mL). After stirring for 2 minutes, succinic acid (1.0 mmol) was added in one portion. The mixture was stirred at room temperature overnight, after which formic acid was added (4.0 eq). The solvent was removed under reduced pressure and the crude material was purified by flash column chromatography using 4 g silica gel column as stationary phase and a mixture of petrol ether (PE) and ethyl acetate (EtOAc), both containing AcOH 0.05% (Flow 15 mL per minute; mixture gradient: from pure PE/AcOH (0.05%) to pure EtOAc/AcOH (0.05% in 20 minutes)) as eluent to afford product as colorless oil in 78% isolated yield.

R_{f}=0.60 (EtOAc/AcOH (0.05%) [KMnO4].

¹H NMR (400 MHz, CDCl₃) δ 4.01 (s, 2H), 3.99 (s, 2H), 3.29 (t, J=6.8 Hz, 2H), 2.72-2.63 (m, 4H), 2.37 (t, J=7.4 Hz, 2H), 1.72-1.58 (m, 4H), 1.47-1.37 (m, 2H).

¹³C NMR (101 MHz, CDCl₃) δ 177.14, 173.00, 171.70, 64.49, 63.82, 51.33, 33.74, 28.78, 28.67, 28.65, 26.29, 24.44.

Step 2) In a syringe containing a filter at the bottom, commercial heterogeneous mixture of aminohexyl agarose (3.0 mL, CAS: 58856-73.8) was introduced (from the top) and the solvent was removed (pushed out from the bottom so no solid material was lost). Then, ultrapure water (3.0 mL) was added (from the bottom) and pushed out (from the bottom). This process was performed 3 times. After that, dried DMF (3.0 mL) was added (from the bottom) and pushed out from the bottom and the procedure was done 3 times. Next, DMF (1.0 mL) was added to suspend the aminohexyl agarose. In parallel, a flask (under argon) with the diazirine linker containing a free carboxylic acid (0.029 mmol), DMAP (3.2 eq) and DMF (2 mL) was stirred for 2 minutes at room temperature. After that, EDC-Cl (3.0 eq) was added and the solution stirred for 10 minutes at room temperature. After that, this solution was introduced to the syringe (from the bottom) containing the suspension of aminohexyl agarose in DMF and the resulting solution was shaken on a shaker at room temperature for 12 h (100 rpm). Aluminium foil was used to protect the syringe from light. After that a flask (under argon) with the linker containing a free carboxylic acid (0.029 mmol), DMAP (3.2 eq) and DMF (2 mL) was stirred for 2 minutes at room temperature. After that, EDC-Cl (3.0 eq) was added and the solution stirred for 10 minutes at room temperature. After that, this solution was introduced to the syringe containing the reaction mixture and it was stirred under the dark for another 12 h. Next, a third time, a flask (under argon) with the linker containing a free carboxylic acid (0.029 mmol), DMAP (3.2 eq) and DMF (2 mL) was stirred for 2 minutes at room temperature. After that, EDC-Cl (3.0 eq) was added and the solution stirred for 10 minutes at room temperature. After that, this solution was introduced to the syringe containing the reaction mixture and it was stirred under the dark for another 12 h. After that, the supernatant was pushed out and the agarose was washed with DMF (3×5.0 mL) and with ultrapure water (3×5.0 mL). After that, the suspension was suspended in 4.0 mL of ultrapure water/EtOH (1:1) and stored in the refrigerator. The incorporation of the linker was confirmed by infra-red spectroscopy, an azide peak was observed at 2362 and 2322 cm⁻¹.

Step 3) In a syringe containing a filter at the bottom, a suspension of solid agarose (attached covalently to the diazirine linker) in 4.0 mL of ultrapure water/EtOH (1:1) (whole batch previously described) was added and the solvent was removed from the bottom. After that, the remaining solid washed with ultrapure water saturated with argon (2×3.0 mL). Then the agarose was suspended in ultrapure water (4.0 mL) followed by addition of an aqueous solution of the peptide (GLP-1 alkyne) (0.004 mmol). After that, CuSO₄ (20.0 eq.) and THTPA (60 eq.) were dissolved in ultrapure water (0.5 mL) and then added to the syringe. Next, aminoguanidin-hydrochloride (50.0 eq) and sodium ascorbate (50.0 eq) was dissolved in 0.5 mL of ultrapure water and was added to the syringe. The mixture was shaken at room temperature for 12 h (100 rpm) and after that, a second addition of the peptide (0.004 mmol), CuSO₄ (20.0 eq) and THTPA (30.0 eq) were repeated and the reaction shaken for another 12 h (100 rpm). Next, the supernatant was pushed out and the agarose was washed with ultrapure water (5×5.0 mL). After that, the agarose was suspended in 4.0 mL of a mixture ultrapure water/EtOH (1:1) and stored in the refrigerator.

It is important to highlight that the structure to be released from the conjugate C is the Exendin-40 free form derivative according to the invention showed in Scheme 17 below.

### Example 8: Synthesis of the GLP-1 Free Form Derivative as Standard for GLP-1 Free Form Derivative Release Studies

In A round-flask equipped with a stir bar under argon, GLP-1 alkyne (0.001 mmol, 5.0 mg) and the commercially available 6-azidohexanoic acid (75.0 eq) were diluted in ultrapure water (2.5 mL) and the resulting solution was stirred for 2 minutes. After that, solid THPTA (120.0 eq.) was added, followed by addition of solid CuSO₄(aq) (60.0 eq). To the resulting solution, solid aminoguanidin-hydrochloride (100.0 eq) was added, followed by addition of solid sodium ascorbate (100.0 eq). To ensure solubility of the organic compounds, tert-butanol (100 μL) was added. After 3 h, the reaction mixture was frozen in liquid nitrogen and placed on the lyophilizer overnight to remove the volatiles. After that, products were diluted in ultrapure water (0.5 mL) and purified by flash column chromatography using C18-reversed phase silica gel as stationary phase and a mixture of water and acetonitrile both containing 0.05% of TFA (mixture gradient: Water (0.05% TFA)/acetonitrile (0.05% TFA) 95:5→5:95) as eluent. The fractions containing the product were identified by LC-MS and the fractions were combined, frozen in liquid nitrogen and placed in lyophilizer overnight to afford product as white solid in 83% isolated yield.

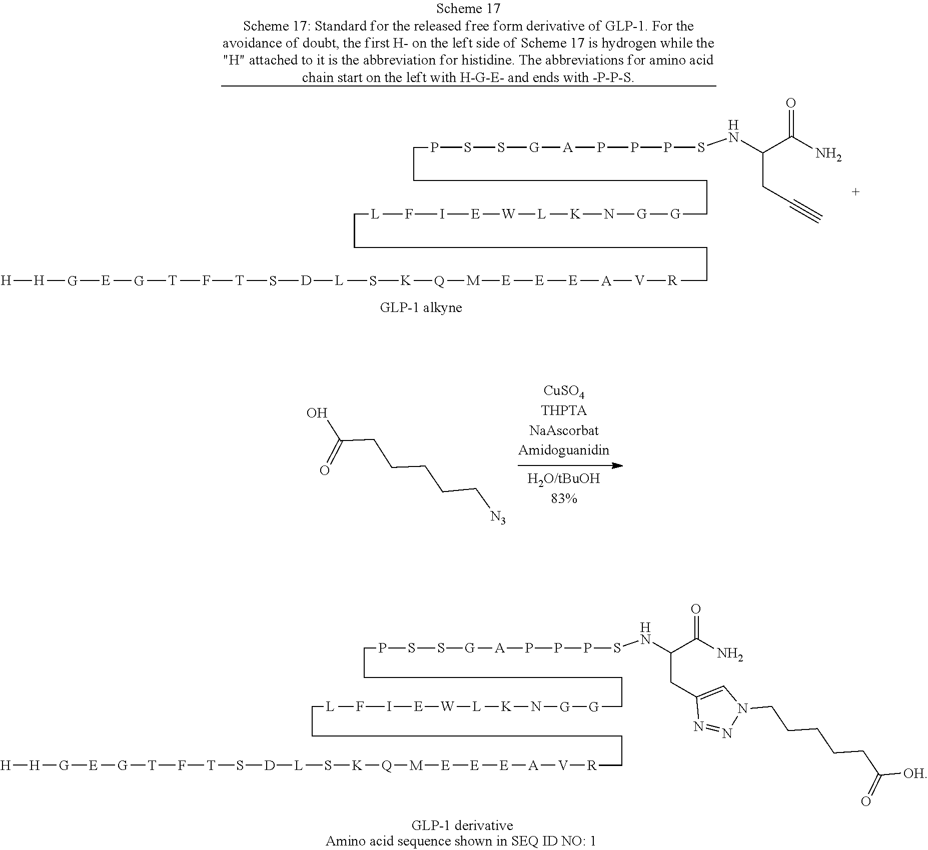

HRMS (ESI) m/z: [C₁₉₅H₂₉₈N₅₄O₆₃S+4H⁺]/4 calcd.: 1110.0452; found: 1110.0452.

### Example 9 Photo Release of Free Form Derivative of GLP-1

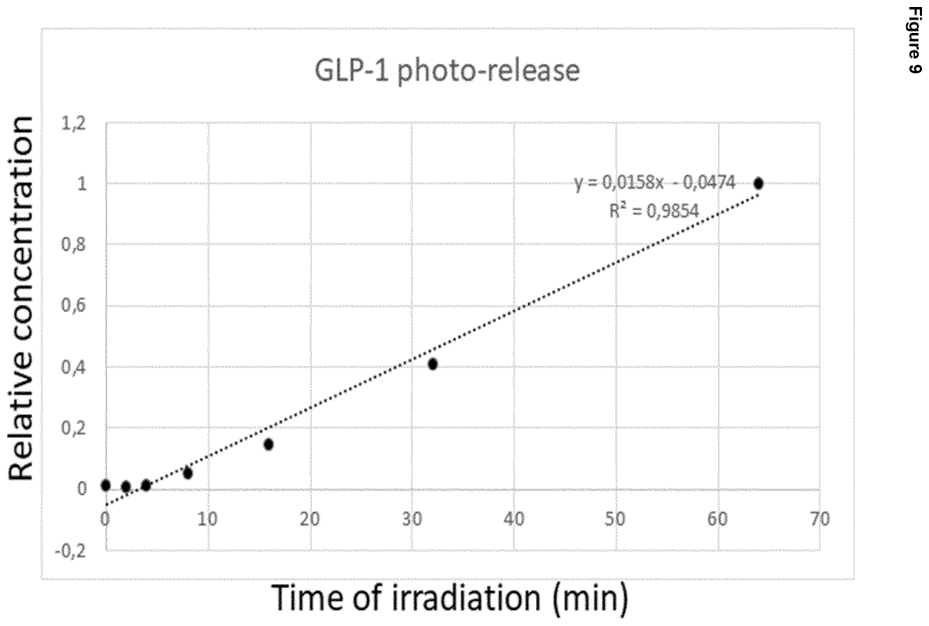

For the photo release of the free form derivative of GLP-1: In a syringe containing a filter at the bottom, the agarose containing the GLP-1 derivative (conjugate C) was washed 3 times with 3.0 mL a HEPES buffer solution (1.0 M). Then 4.0 mL of HEPES buffer solution were added and the heterogeneous mixture was placed in a 96-well plate (100 μL per well) After that, before irradiation, 3 samples (time zero) were removed and placed in 3 LC-MS vials using a syringe with a filter to prevent agarose from going to the vial. Then, the 96-well plate was placed under a UV light and irradiation was initiated. For each indicated time of irradiation (from 0 min, 0.5 min, 1 min, 2 min, 4 min, 8 min, 16, min, 32 min and 64 min), samples were taken (60 μL) for LC-MS analysis (in triplicates), which were always filtered before addition to the LC-MS vials. The results were analyzed by UPLC-MS and the integration values (averages of triplicates) of the photo release of a GLP-1 free form derivative upon UV irradiation are shown in FIG. 9.

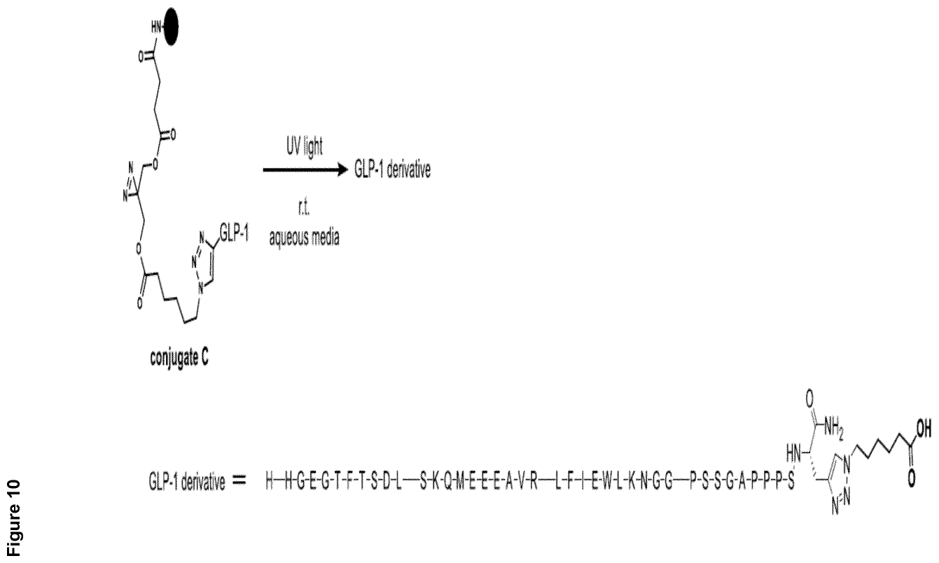

The released free form derivative of GLP-1 from the conjugate according to the invention is schematically shown in FIG. 10.

Relative concentration of GLP-1 derivative determined by UPLC-MS correlated to the time of irradiation with UV light. Integrals were calculated by selecting the masses which corresponds to the various degree of protonation of the GLP-1 free form derivative (888.46 and 1110.35 m/z, abs window Da=0.15) in the retention time between 1.4-2.0 minutes.

Liberation of GLP-1 free form derivative could be observed after 8 minutes of irradiation. Albeit the sensitivity of the detection method was limited, we could show a linear release of GLP-1 free form derivative. Further experiments were done using insulin and glucagon derivatives and the results are shown below.

### Example 10: Synthesis of Drug-Linker-Polymer Conjugate of Formula (A1) with Glucagon Via a Glucagon-Alkyne Derivative

Glucagon is frequently used in emergency medical treatment to treat hypoglycemia. It is a drug that can be released upon light using the linker described in this invention. Similar to the protocol done for Exedin-40, a glucagon-alkyne was attached to a linker-agarose conjugate following the same protocol as previously.

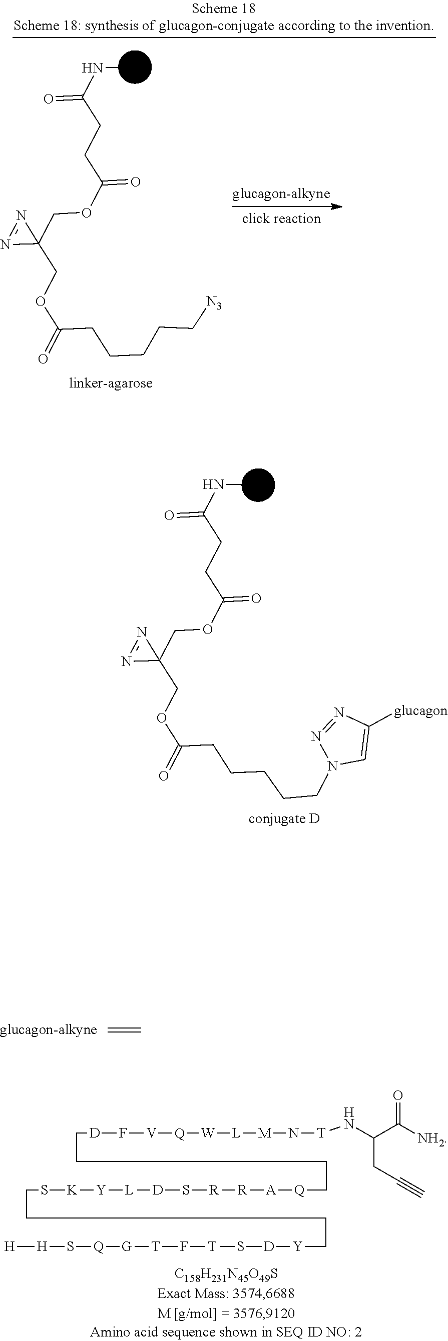

In a syringe containing a filter at the bottom, a suspension of solid agarose (attached covalently to the diazirine linker) in in 4.0 mL of ultrapure water/EtOH (1:1) (whole batch previously described) was added and the solvent was removed from the bottom. After that, the remaining solid was washed with ultrapure water saturated with argon (2×3.0 mL). Then the agarose was suspended in ultrapure water (4.0 mL) followed by addition of an aqueous solution of the peptide (glucagon-alkyne, scheme 18) (0.004 mmol). After that, CuSO₄ (20.0 eq.) and THTPA (60 eq.) were diluted in ultrapure water (0.5 mL) and then it was added to the syringe. Next, aminoguanidin-hydrochloride (50.0 eq) and sodium ascorbate (50.0 eq) was diluted in 0.5 mL of ultrapure water and was added to the syringe. The mixture was shaken at room temperature for 12 h (100 rpm) and after that, a second batch of the peptide (0.001 mmol), CuSO₄ (20.0 eq) and THTPA (30.0 eq) were added and the reaction shaken for another 12 h (100 rpm). Next, the supernatant was pushed out and the agarose was washed with ultrapure water (5×5.0 mL). After that, the agarose was suspended in 4.0 mL of a mixture ultrapure water/EtOH (1:1) and stored in a refrigerator.

The structure to be released from the conjugate D is the glucagon derivative shown in Scheme 19 below.

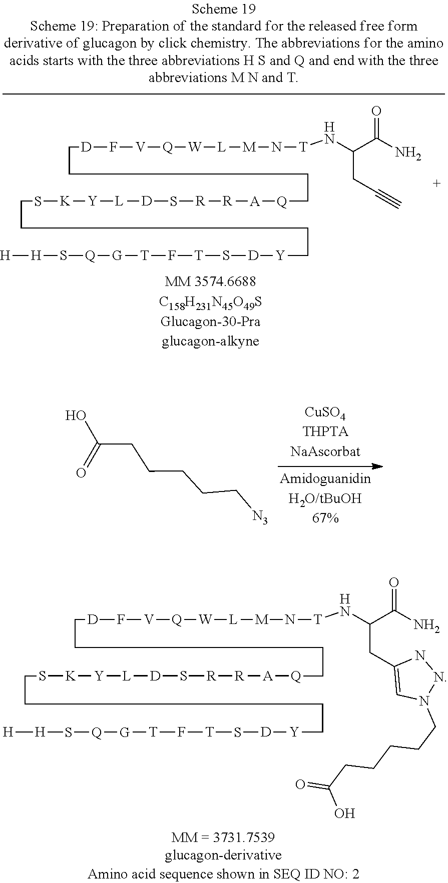

In a round-flask equipped with a stir bar under argon, glucagon-alkyne (0.001 mmol, 4.6 mg) and the commercially available 6-azidohexanoic acid (75.0 eq) were diluted in ultrapure water (2.5 mL) and the resulting solution was stirred for 2 minutes. After that, solid THPTA (120.0 eq.) was added, followed by addition of solid CuSO₄(aq) (60.0 eq). To the resulting solution, solid aminoguanidin-hydrochloride (100.0 eq) was added, followed by addition of solid sodium ascorbate (100.0 eq). To ensure solubility of the organic compounds, tert-butanol (100 μL) was added. After 3 h, the reaction mixture was frozen in liquid nitrogen and placed on the lyophilizer overnight to remove the volatiles. After that, products were diluted in ultrapure water (0.5 mL) and purified by flash column chromatography using C18-reversed phase silica gel as stationary phase and a mixture of water and acetonitrile both containing 0.05% of TFA (mixture gradient: Water (0.05% TFA)/acetonitrile (0.05% TFA) 95:5→5:95) as eluent. The fractions containing the product were identified by LC-MS and the fractions were combined, frozen in liquid nitrogen and placed in lyophilizer overnight to afford product as white solid in 67% isolated yield.

HRMS (ESI) m/z: [C₁₆₄H₂₄₂N₄₈O₅₁S+5H⁺]/5 calcd.: 747.3586; found: 747.3589.

The product of this click reaction was used as standard to do the calibration curve of the release experiment.

### Example 11: Photo Release of the Free Form Derivative of Glucagon

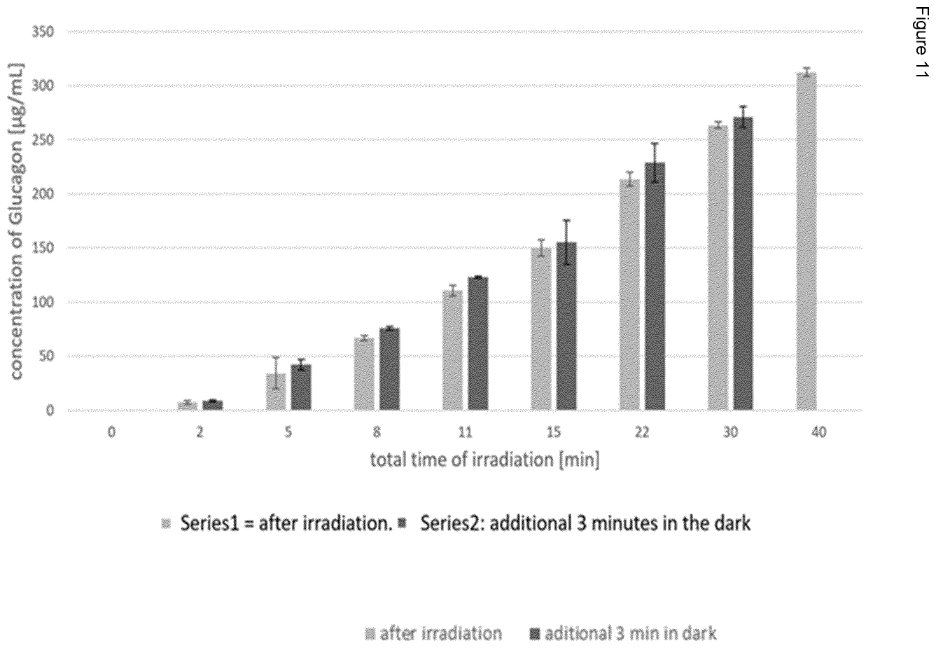

For the photo release of glucagon: to a syringe containing a filter at the bottom, the agarose containing the glucagon derivative (conjugate D) was washed 3 times with 3.0 mL ultra-pure water. Then it was added 5.0 mL of ultra-pure water (containing 0.05% of acetic acid) and the heterogeneous mixture was placed in a 96-well plate (110 μL per well, 44 wells were used). After that, before irradiation, 3 samples (time zero) were removed and placed to 3 LC-MS vials using a syringe with a filter to prevent agarose from going to the vial. Then, the 96-well plate was placed under a UV light and irradiation was initiated. For each appropriate time of irradiation (from 0 min, 2.0 min, 5.0 min, 8.0 min, 15.0 min, 22.0 min, 30.0, min, 40 min), 3 samples were taken (100 μL) for LC-MS analysis (in triplicates) right after the light was turned off. Before the initiation of the next round of irradiation, the plate was kept for 3 minutes in the dark and after that, another 3 samples were taken (100 μL) for LC-MS analysis. All samples were filtered before addition to the LC-MS vials. Note that for each time of irradiation 6 wells were analyzed: 3 right after irradiation and 3 after keeping the mixture for additional 3 minutes in the dark. FIG. 11 shows the concentration of the released free form derivative of glucagon which was identified with the same UPLC-MS methods as for the free form derivative of GLP-1 (see Examples 7 to 9) correlated to the time of irradiation with UV light (calibration curve was done with the synthesized glucagon derivative). Integrals were calculated by selecting the masses which corresponds to the various degree of protonation of the glucagon derivative (748; 934.8 and 1246 m/z, abs window Da=2.0) in the retention time between 1.0-1.5 minutes.

With this optimized irradiation protocol we observed a clear correlation of the amount of glucagon released and the duration of UV light exposure. The additional 3 minutes in the dark between analysis time points, shows that the release of the peptide does not continue after the light irradiation is stopped.

### Example 12: Synthesis of Drug-Linker-Polymer Conjugate of Formula (A1) with Insulin

Due to the importance of insulin as a pharmaceutical, the linker system described on this invention was used to photo release this drug in one of its free form derivatives.

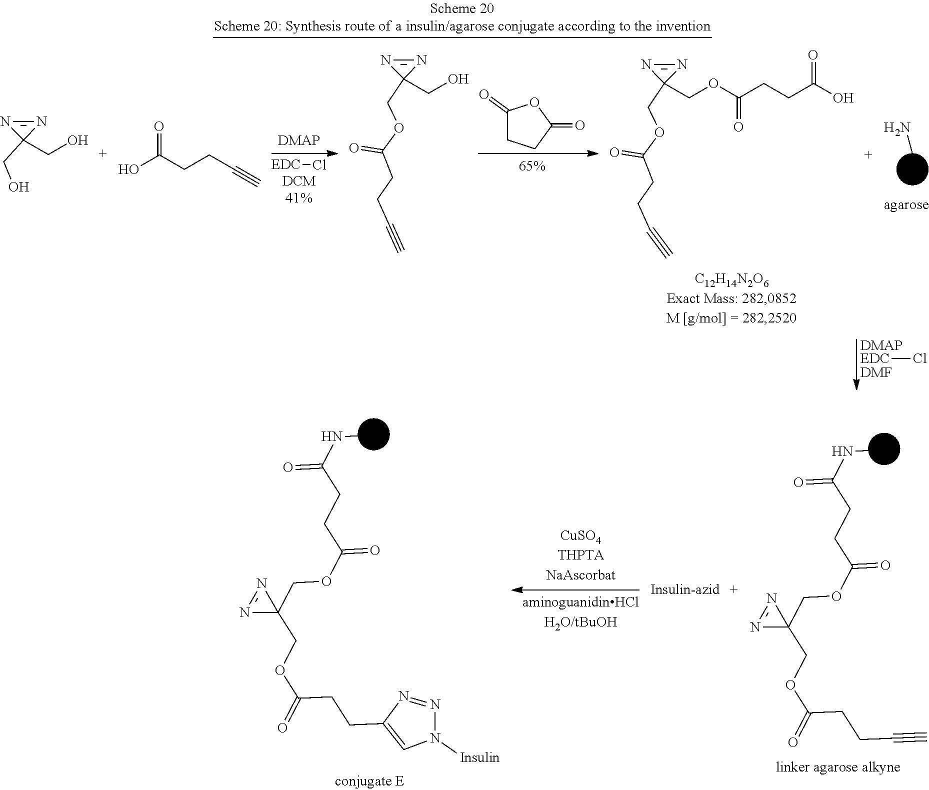

1) In a round-flask equipped with a stir bar under argon, was added (3-(hydroxymethyl)-3H-diazirin-3-yl)methyl pent-4-ynoate (0.55 mmol) and DMAP (1.2 eq) followed by dilution with DCM (3 mL). After stirring for 2 minutes, succinic acid (1.0 mmol) was added in one portion. The mixture was stirred at room temperature overnight, after which formic acid was added (4.0 eq). The solvent was removed under reduced pressure and the crude was purified by flash column chromatography using 4 g silica gel column as stationary phase and a mixture of petrol ether (PE) and ethyl acetate (EtOAc) both containing 0.05% of acetic acid (flow 15 mL per minute, mixture gradient: from pure PE/AcOH (0.05%) to pure EtOAc/AcOH (0.05% in 20 minutes) as eluent to afford product (4-oxo-4-((3-((pent-4-ynoyloxy)methyl)-3H-diazirin-3-yl)methoxy)butanoic acid) as white solid in 65% isolated yield.

R_{f}=0.54 (EtOAc/AcOH (0.05%) [KMnO4].

¹H NMR (400 MHz, CDCl₃) δ 4.02 (d, J=2.0 Hz, 4H), 2.73-2.64 (m, 4H), 2.61 (dd, J=10.7, 4.1 Hz, 2H), 2.52 (td, J=6.8, 2.0 Hz, 2H), 2.00 (t, J=2.5 Hz, 1H).

¹³C NMR (101 MHz, CDCl₃) b 176.63, 171.72, 170.70, 82.23, 69.48, 64.43, 64.28, 33.12, 28.70, 28.51, 14.43.

HRMS (ESI) m/z: [C₁₂H₁₄N₂O₆+Na]⁺ calcd.: 305.0750; found: 305.0750.

2) In a syringe containing a filter at the bottom, commercial heterogeneous solution of aminohexyl agarose (3.0 mL, CAS: 58856-73.8) was introduced (from the top) and the original solution was removed (pushed out from the bottom so no solid material was lost). Then, ultrapure water (3.0 mL) was added (from the bottom) and pushed out (from the bottom). This process was performed 3 times. After that, dried DMF (3.0 mL) was added (from the bottom) and pushed out from the bottom and the procedure was done 3 times. Next, DMF (1.0 mL) was added to suspend the aminohexal agarose. In parallel, a flask (under argon) with the linker 4-oxo-4-((3-((pent-4-ynoyloxy)methyl)-3H-diazirin-3-yl)methoxy)butanoic acid (0.035 mmol), DMAP (3.2 eq) and DMF (2 mL) was stirred for 2 minutes at room temperature. After that, EDC-Cl (3.0 eq) was added and the solution stirred for 10 minutes at room temperature. After that, this solution was introduced to the syringe (from the bottom) containing the suspension of aminohexyl agarose in DMF and the resulting solution was shaken on a shaker at room temperature for 12 h (100 rpm). PS: aluminium foil was used to protect the syringe from light. After that, once again, a flask (under argon) with the linker 4-oxo-4-((3-((pent-4-ynoyloxy)methyl)-3H-diazirin-3-yl)methoxy)butanoic acid (0.035 mmol), DMAP (3.2 eq) and DMF (2 mL) was stirred for 2 minutes at room temperature. After that, EDC-Cl (3.0 eq) was added and the solution stirred for 10 minutes at room temperature. After that, this solution was introduced to the syringe containing the reaction mixture and it was stirred under the dark for another 12 h. Next, a third time, a flask (under argon) with the linker 4-oxo-4-((3-((pent-4-ynoyloxy)methyl)-3H-diazirin-3-yl)methoxy)butanoic acid (0.035 mmol), DMAP (3.2 eq) and DMF (2 mL) was stirred for 2 minutes at room temperature. After that, EDC-Cl (3.0 eq) was added and the solution stirred for 10 minutes at room temperature. After that, this solution was introduced to the syringe containing the reaction mixture and it was stirred under the dark for another 12 h. After that, the supernatant was pushed out and the agarose was washed with DMF (3×5.0 mL) and with ultrapure water (3×5.0 mL). After that, the suspension was suspended in 4.0 mL of ultrapure water/EtOH (1:1) and stored in the refrigerator and used in the next step.

3) In a syringe containing a filter at the bottom, a suspension of solid agarose (linker agarose alkyne) in in 4.0 mL of ultrapure water/EtOH (1:1) (whole batch previously described) was added and the solvent was removed from the bottom. After that, the remaining solid was washed with ultrapure water saturated with argon (2×3.0 mL). Then the agarose was suspended in ultrapure water (4.0 mL) followed by addition of an aqueous solution of the insulin azide (mono-functionalized, obtained by reacting human insulin with 3-azidobutanoic acid N-hydroxysuccinimide ester (see general information), 0.005 mmol). After that, CuSO₄ (20.0 eq.) and THTPA (60 eq.) were diluted in ultrapure water (0.5 mL) and then it was added to the syringe. Next, aminoguanidin-hydrochloride (50.0 eq) and sodium ascorbate (50.0 eq) was diluted in 0.5 mL of ultrapure water and was added to the syringe. The mixture was shaken at room temperature for 24 h (100 rpm) and after that, the supernatant was pushed out and the agarose was washed with ultrapure water (5×5.0 mL). After that, the agarose was suspended in 4.0 mL of a mixture ultrapure water/EtOH (1:1) and stored in the refrigerator.

### Example 13: Synthesis of the Insulin Free Form Derivative as Standard for Insulin Free Form Derivative Release Studies

A standard to prepare the calibration curve for the photorelease analysis was performed according to Scheme 21.

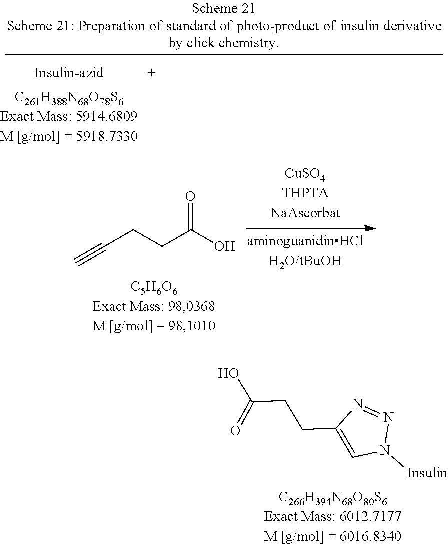

In a round-flask equipped with a stir bar under argon, insulin-azide (mono-functionalized, obtained by reacting human insulin with 3-azidobutanoic acid N-hydroxysuccinimide ester, 0.0001 mmol, 0.75 mg) and the commercially available pent-4-ynoic acid (75.0 eq) were diluted in ultrapure water (2.5 mL) and the resulting solution was stirred for 2 minutes. After that, solid THPTA (120.0 eq.) was added, followed by addition of solid CuSO₄(aq) (60.0 eq). To the resulting solution, solid aminoguanidin-hydrochloride (100.0 eq) was added, followed by addition of solid sodium ascorbate (100.0 eq). To ensure solubility of the organic compounds, tert-butanol (100 μL) was added. After 3 h, the reaction mixture was frozen in liquid nitrogen and placed on the lyophilizer overnight to remove the volatiles. After that, products were diluted in ultrapure water (0.5 mL) and purified by flash column chromatography using C18-reversed phase silica gel as stationary phase and a mixture of water and acetonitrile both containing 0.05% of TFA (mixture gradient: Water (0.05% TFA)/acetonitrile (0.05% TFA) 95:5→5:95) as eluent. The fractions containing the product were identified by LC-MS and the fractions were combined, frozen in liquid nitrogen and placed in lyophilizer overnight to afford product as white solid in quantitative yield.

HRMS (ESI) m/z: [C₂₆₆H₃₉₄N₆₈O₈₀S₆+5H⁺]/5 calcd.: 1202.5435; found: 1202.6327.

### Example 14: Photo Release of the Free Form Derivative of Insulin

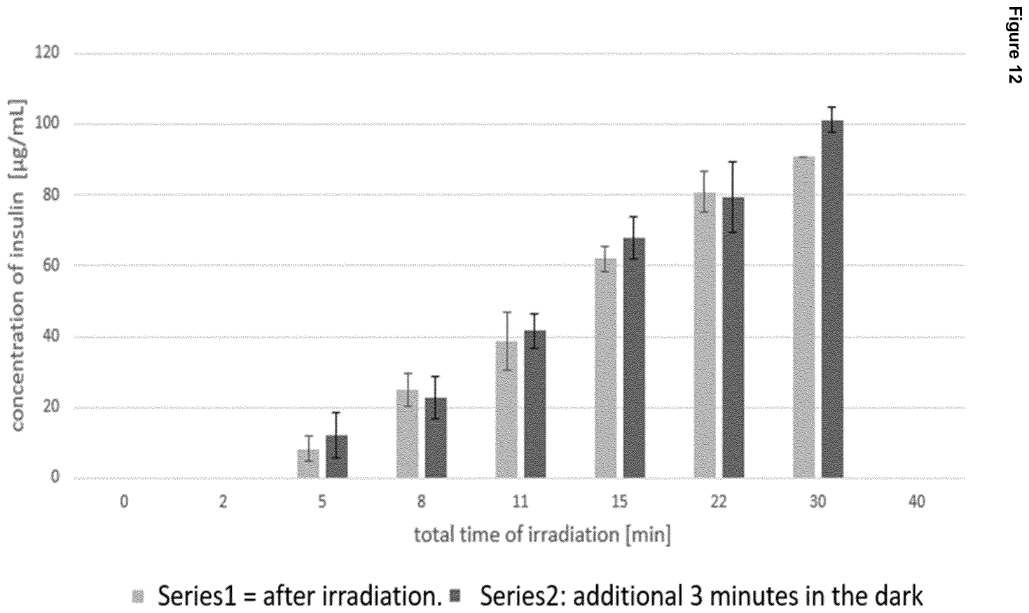

For the photo release of a free form derivative of insulin: to a syringe containing a filter at the bottom, the agarose containing the insulin derivative (conjugate E) was washed 3 times with 3.0 mL ultra-pure water. Then it was added 5.0 mL of ultra-pure water (containing 0.05% of acetic acid) and the heterogeneous solution was placed in a 96-well plate (110 μL per well, 44 wells were used). After that, before irradiation, 3 samples (time zero) were removed and placed to 3 LC-MS vials using a syringe with a filter to keep agarose from the vial. Then, the 96-well plate was placed under a UV light and irradiation was initiated. For each appropriated time of irradiation (from 0 min, 2.0 min, 5.0 min, 8.0 min, 15.0 min, 22.0 min, 30.0, min, 40 min), 3 samples were taken (100 μL) for LC-MS analysis (in triplicates) right after the light was turned off. Before the initiation of the next round of irradiation, it was waited 3 minutes in the dark and after that, another 3 samples were taken (100 μL) for LC-MS analysis, which were always filtered before addition to the LC-MS vials. PS: for each time of irradiation 6 wells was analyzed: 3 right after irradiation and 3 after 3 minutes in the dark. After the irradiation procedure, the samples were analyzed by UPLC-MS and the integration values (triplicates averages) corresponding to the peptide sign are shows in FIG. 12. FIG. 12 shows the concentration of the released insulin derivative determined by UPLC-MS correlated to the time of irradiation with UV light (calibration curve was done with the synthesized insulin derivative). Integrals were calculated by selecting the masses which corresponds to the various degree of protonation of the liberated insulin derivative (1003 (M+6/6) and 1203 ((M+5)/5) m/z, abs window Da=0.2) in the retention time between 1.1-1.6 minutes.

As for all the other examples, it can be seen the drug being release upon light irradiation.

### Example 15: (3-(((methylsulfonyl)thio)methyl)-3H-diazirin-3-yl)methyl pent-4-ynoate

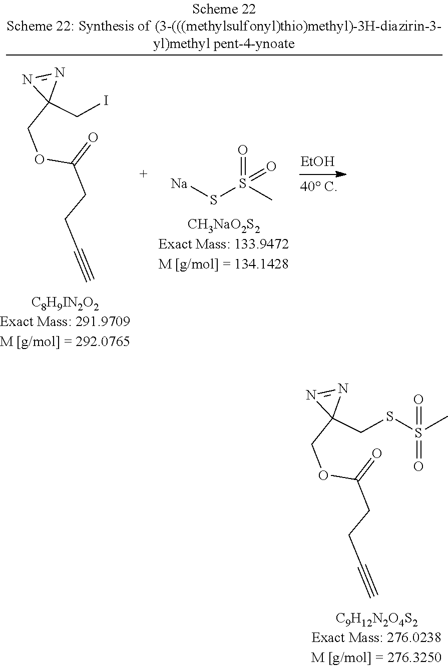

Under argon, derivative (3-(iodomethyl)-3H-diazirin-3-yl)methyl pent-4-ynoate (29 mg, 0.10 mmol, 1.0 eq) was solubilized in dry ethanol (1.0 mL). The solution was stirred for 5 min at r.t. and after that sodium methanethiosulfonate (15 mg, 0.11 mmol, 1.1 eq) was added at once. The resulting mixture was stirred at r.t. for 3 h and after that the crude was diluted with water (2.0 mL) and the resulting solution was extracted with DCM (3×5.0 mL). The combined organic layers were dried over Na₂SO₄ and the solvent was evaporated in vacuo to afford product (27 mg, 0.098 mmol, 98%) as red film.

R_{f}=0.29 (Petrol ether/EtOAc (3:1) [KMnO4]).

¹H NMR (400 MHz, CDCl₃) δ 5.30 (s, 2H), 4.06 (s, 2H), 3.47 (s, 2H), 3.31 (bs, 3H), 2.62 (dd, J=11.0, 4.0 Hz, 2H), 2.57-2.49 (m, 2H), 2.01 (t, J=2.5 Hz, 1H).

¹³C NMR (101 MHz, CDCl₃) b 171.33, 82.16, 69.58, 64.79, 51.39, 39.14, 33.11, 14.42.

HRMS (ESI) m/z: [C₉H₁₂N₂O₄S₂+Na]⁺ calcd.: 299.0136; found: 299.0128

### Example 16: tert-butyl ((3-(2-((tert-butoxycarbonyl)amino)ethyl)-3H-diazirin-3-yl)methyl)carbamate

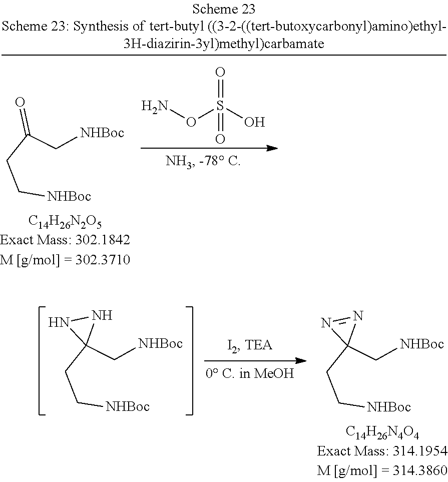

100 mL of dry ammonia gas was condensed in a three-way flask at −78° C. in a dry ice/acetone bath. Di-tert-butyl (2-oxobutane-1,4-diyl)dicarbamate¹ (300 mg, 0.992 mmol, 1.0 eq) were dissolved in 5.0 ml dry methanol and added to liquid ammonia and stirred for 3 h. Then, Hydroxylamine O-sulfonic acid (112 mg, 0.992 mmol, 1.0 eq) was dissolved in 5.0 ml dry methanol and added drop wise to the mixture over the course of 1 min. During this time an overpressure of ammonia was maintained and the reaction mixture cooled to −78° C. After that the mixture was allowed to reach −60° C. and stirred for 2 h. After that, the mixture was stirred over night while the dry ice bath evaporated and the mixture slowly reached room temperature. On the next day the remaining ammonia was removed by bubbling nitrogen through the mixture, the white precipitate was filtered off (washed with methanol, ca 50 mL) and the methanol was evaporated to obtain the intermediate diaziridine. Without further purification, the diaziridine was oxidized to diazirine. For this, methanol (10.0 mL) and triethylamine (151 mg, 207 μL, 1.49 mmol, 1.5 eq.) were added to the crude intermediate at 0° C. Under stirring, solid iodine was added slowly until the mixture stayed red for over 5 min (459 mg was needed). The reaction was stirred for an additional 2 h at room temperature, after which the methanol was evaporated and the mixture was extracted with ethyl acetate (50 mL) and dried over sodium sulphate. The ethyl acetate was evaporated under reduced pressure and the crude product purified by flash chromatography using PE/EtOAc as eluent. ¹ This starting material was prepared according to the described in the literature: Hwang et al Applied Radiation and Isotopes (1986), 37(7), 607-612.

R_{f}=0.64 (Petrol ether/EtOAc (1:1) [KMnO4]).

¹H NMR (400 MHz, CDCl₃) δ 4.95 (s, 1H), 4.53 (s, 1H), 3.15 (d, J=5.6 Hz, 2H), 3.06 (s, 2H), 1.60 (t, J=6.5 Hz, 2H), 1.44 (d, J=5.6 Hz, 18H).

HRMS (ESI) m/z: [C₁₄H₂₆N₄O₄+H]⁺ calcd.: 315.2032; found: 315.2031.

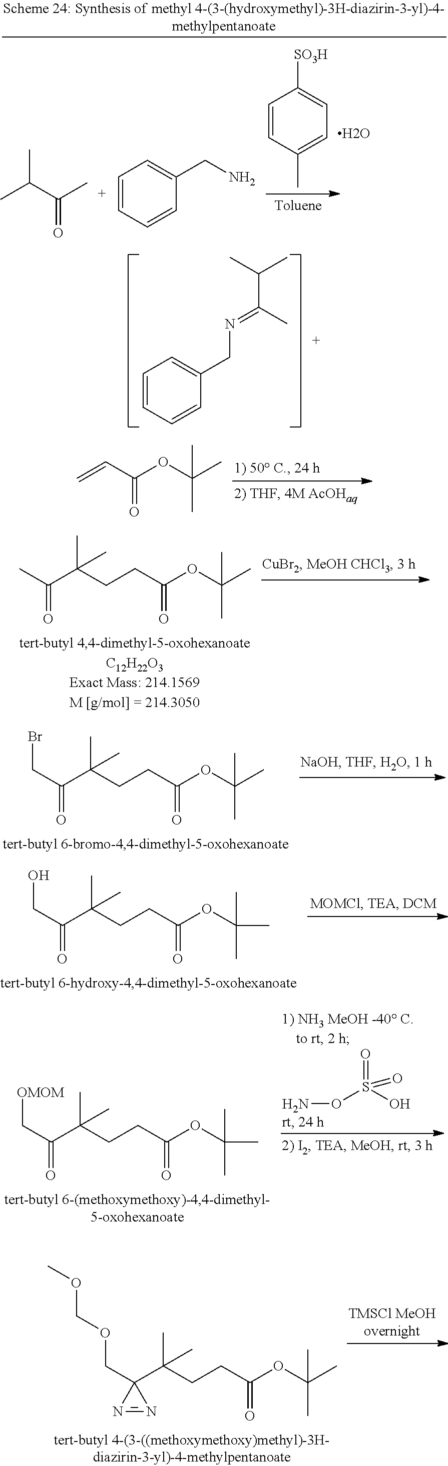

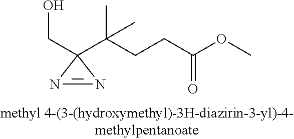

### Tert-butyl 4,4-dimethyl-5-oxohexanoate

A solution of isopropyl methyl ketone (10 g, 116 mmol, 1 eq) benzylamine (13.7 g, 128 mmol, 1.10 eq), and p-toluenesulfonic acid (442 mg, 2 mol %) was heated in toluene (129 mL 0.9 M) at reflux on a Dean-Stark apparel for 2.5 hours. The solvent was removed under reduced pressure to obtain (E)-N-benzyl-3-methylbutan-2-imine as light yellow oil, which was used in the next step without purification. The above obtained imine (20 g, 116 mmol) was dissolved in tert-butyl acrylate (16.4 g 128 mmol, 1.1 eq.). The mixture was heated at 50° C. and stirred for 15 hours. The reaction mixture was diluted in tetrahydrofurane-4M acetic acid (2:1, 140 mL) and stirring was continued for another 24 hours. The reaction mixture was extracted with dichloromethane (3×100 mL). The organic phase was dried over sodium sulphate, evaporated to dryness and the residue was purified by vacuum distillation to afford tert-butyl 4,4-dimethyl-5-oxohexanoate (11 g, 46%).

TLC: PE/EA=25/1, R_{f}=0.2

LC-MS: default unpol, RT=1.289 min, found 237.1 [M+Na]+

¹H-NMR (CDCl3 400 MHz) δ 1.12 (6H, s), 1.43 (9H, s), 1.80-1.84 (2H, m), 2.09-2.14 (5H, m).

¹³C-NMR (CDCl3 101 MHz) δ 24.26, 25.21, 28.21, 31.22, 34.44, 47.30, 80.54, 172.90, 213.46.

HR-MS: C₁₂H₂₂O₃ calculated [M+Na]⁺ 237.1467 found 237.1464

### Tert-butyl 6-bromo-4,4-dimethyl-5-oxohexanoate

Tert-butyl 6-bromo-4,4-dimethyl-5-oxohexanoate (3 g, 14 mmol, 1 eq.), CuBr₂ (9.4 g, 42 mmol, 3 eq.) and methanol/chloroform (1:1 volume ratio, 140 mL) were added to a three-necked flask and stirred at 40° C. for 3 h, note that CuBr₂ was added in four portions over 30 mins. After stirring for 3 h, the mixture was diluted with petroleum ether and washed with water. After drying over sodium sulphate, the mixture was concentrated under vacuum to afford tert-butyl 6-bromo-4,4-dimethyl-5-oxohexanoate, which was used in next step without purification.

TLC: PE/EA=25/1, R_{f}=0.29

LC-MS: default, RT=1.495 min, found 315.0 [M+Na]+

### Tert-butyl 6-hydroxy-4,4-dimethyl-5-oxohexanoate

70 ml 1 M NaOH was added into a THF (70 ml) solution of the above obtained tert-butyl 6-bromo-4,4-dimethyl-5-oxohexanoate and the mixture was allowed to stir for 60 mins. After quenching with sat. NH₄Cl aq, it was extracted with ethyl acetate. After drying over sodium sulphate, the solvent was removed under vacuum and the residue was purified by silica column to afford tert-butyl 6-hydroxy-4,4-dimethyl-5-oxohexanoate (2.2 g, 68% over 2 steps)

TLC: PE/EA=9/1, R_{f}=0.14

LC-MS: default unpol, RT=0.164 min, found 253.1 [M+Na]+

¹H-NMR (DMSO 400 MHz) δ 1.05 (6H, s), 1.38 (9H, s), 1.67-1.70 (2H, m), 2.03-2.07 (2H, m), 4.29 (2H, d, J=5.81 Hz), 4.65 (H, t, J=5.81 Hz).

¹³C-NMR (DMSO 101 MHz) δ 23.43, 27.72, 30.46, 34.01, 44.64, 63.87, 79.64, 172.04, 214.12.

HR-MS: C₁₂H₂₂O₄ calculated [M+Na]⁺ 253.1416 found 253.1415

### Tert-butyl 6-(methoxymethoxy)-4,4-dimethyl-5-oxohexanoate

To a mixture of tert-butyl 6-hydroxy-4,4-dimethyl-5-oxohexanoate (500 mg, 2.2 mmol, 1 eq.) and diisopropyl ethyl amine (421 mg, 3.3 mmol, 1.5 eq) in dichloromethane (6 mL) was added MOMCl (227 mg, 2.8 mmol, 1.3 eq) at 0° C., and the mixture was stirred at room temperature overnight. After it was diluted with water, the mixture was extracted with DCM (3×10 mL) and the combined organic layers were washed with brine (8 mL) and dried over sodium sulphate. After the solvent was removed in vacuo, the crude product was filtered through a silica bed using 20% EtOAc/80% hexanes to afford tert-butyl 6-(methoxymethoxy)-4,4-dimethyl-5-oxohexanoate (481 mg, 96%).

TLC: PE/acetone=9/1, R_{f}=0.27

LC-MS: default, RT=1.284 min, found 297.1 [M+Na]+

¹H-NMR (CDCl₃ 400 MHz) δ 1.15 (6H, s), 1.43 (9H, s), 1.81-1.85 (2H, m), 2.12-2.16 (2H, m), 3.38 (2H, s), 4.39 (2H, s), 4.68 (2H, s).

¹³C-NMR (CDCl₃ 101 MHz) δ 24.07, 28.20, 31.04, 34.50, 45.91, 55.86, 68.16, 80.63, 96.57, 172.71, 210.51.

HR-MS: C₁₄H₂₆O₅ calculated [M+Na]⁺ 297.1678 found 297.1683

### Tert-butyl 4-(3-((methoxymethoxy)methyl)-3H-diazirin-3-yl)-4-methylpentanoate

To liq. ammonia, tert-butyl 6-(methoxymethoxy)-4,4-dimethyl-5-oxohexanoate (500 mg, 1.82 mmol) in 2 mL methanol was added at −78 degree and then stirred for 2 h with allowing the temperature to slowly warm from −78 degree to room temperature. Then hydroxylamine-O-sulfonic acid (309 mg, 2.73 mmol) in 2 mL dry methanol was added and stirring at room temperature was continued overnight. Argon was bubbled into the solution to remove the remained NH₃ and the solids were filtered out. Triethyl amine (277 mg, 2.73 mmol) was added to the filtrate at 0° C., followed by addition of iodine (509 mg, 2 mmol). After stirring for 3 h, the reaction mixture was diluted with diethyl ether, washed with water and sat. Na₂S₂O₃ and dried over sodium sulphate. After concentration of the organic layer under vacuum, the residue was purified by silica gel chromatography to afford tert-butyl 4-(3-((methoxymethoxy)methyl)-3H-diazirin-3-yl)-4-methylpentanoate (104 mg, 20%).

TLC: PE/EA=9/1, R_{f}=0.50

LC-MS: default unpol, RT=0.338 min, found 309.1 [M+Na]+

¹H-NMR (CDCl₃ 400 MHz): 0.75 (6H, s), 1.45 (9H, s), 1.58-1.62 (2H, m), 2.35-2.39 (2H, m), 3.30 (2H, s), 3.47 (2H, s), 4.47 (2H, s).

¹³C-NMR (CDCl₃ 101 MHz): 24.90, 28.24, 30.77, 32.48, 34.50, 35.05, 55.56, 65.54, 80.47, 96.21, 173.14.

HR-MS: C₁₄H₂₆N₂O₄ calculated [M+Na]⁺ 309.1790 found 309.1779

### Methyl 4-(3-(hydroxymethyl)-3H-diazirin-3-yl)-4-methylpentanoate

Trimethylsilyl chloride (66 μL, 0.52 mmol, 3.00 equiv) was added dropwise to a solution of tert-butyl 4-(3-((methoxymethoxy)methyl)-3H-diazirin-3-yl)-4-methylpentanoate (50 mg, 0.17 mmol, 1 eq) in methanol (2 mL) at 0° C. The reaction mixture was stirred over night at room temperature, after which the reaction mixture was evaporated to dryness. The resulting residue was purified by silica column to afford methyl 4-(3-(hydroxymethyl)-3H-diazirin-3-yl)-4-methylpentanoate (39 mg, 93%)

TLC: PE/EA=4/1, R_{f}=0.30

¹H-NMR (CDCl₃ 400 MHz) δ 0.76 (6H, s), 1.45 (1H, d, J=5.34 Hz), 1.66-1.70 (2H, m), 2.44-2.48 (2H, m), 3.59 (2H, d, J=5.69 Hz), 3.71 (3H, s).

¹³C-NMR (CDCl₃ 101 MHz) δ 24.98, 29.42, 33.86, 34.46, 35.07, 52.01, 61.24, 174.71.

HR-MS: C₁₄H₂₆N₂O₄ calculated [M+Na]⁺ 223.1059 found 223.1059

1. A drug conjugate of formula (A1) 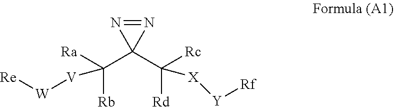
wherein
Ra and Rb independently from each other represents a moiety selected from H, halogen, (C₁-C₅)alkyl, (C₂-C₅)alkenyl, a five- or six-membered heterocycle, (C₃₋₃₀)cycloalkyl, phenyl, benzyl (—CH₂—C₆H₅) each heterocycle, cycloalkyl, phenyl or benzyl can optionally be substituted with 1, 2, 3, 4 or 5 substituents selected from the group consisting of hydroxy, halogen, (C₁-C₅)alkyl, (C₁-C₅)alkoxy (—O—(C₁-C₅)alkyl); or
Ra and Rb form together with the carbon they are attached to a five- or six-membered heterocycle or (C₃₋₁₂)cycloalkyl, preferably cyclopentyl or cyclohexyl optionally substituted with 1, 2, 3, 4 or 5 substituents selected from the group consisting of hydroxy, halogen, (C₁-C₅)alkyl, (C₁-C₅)alkoxy); or
Ra and Rb represent together with the carbon they are attached to and V a (C₃₋₃₀)cycloalkyl moiety to which —W—Re is attached wherein the cycloalkyl moiety is selected from the group consisting of adamantyl (C₁₀H₁₄), iceanyl (C₁₂H₁₆), diadamantanyl (C₁₄H₁₈), triadamantyl (C₂₂H₂₂), isotetramantanyl (C₂₂H₂₆), pentamantanyl (C₂₆H₃₀), cyclohexamantanyl (C₂₆H₂₈), superadamantan (C₃₀H₃₄), each optionally substituted with 1, 2, 3, 4 or 5 substituents selected from the group consisting of hydroxy, halogen, (C₁-C₅)alkyl, (C₁-C₅)alkoxy; more preferably the cycloalkyl is selected from the group consisting of adamantyl (C₁₀H₁₄), iceanyl (C₁₂H₁₆), diadamantanyl (C₁₄H₁₈), triadamantyl (C₂₂H₂₂), isotetramantanyl (C₂₂H₂₆), pentamantanyl (C₂₆H₃₀), cyclohexamantanyl (C₂₆H₂₈), superadamantan (C₃₀H₃₄), more preferably the cycloalkyl is selected from the group consisting of adamantyl (C₁₀H₁₄), iceanyl (C₁₂H₁₆), diadamantanyl (C₁₄H₁₈), even more preferably adamantyl (C₁₀H₁₄)); or
Ra and Rb form together with the carbon they are attached to and V a phenyl moiety to which —W—Re is attached;
Rc and Rd independently from each other represents a moiety selected from H, hydroxy, halogen selected from the group consisting of F, Cl, I or Br), (C₁-C₅)alkyl, (C₂-C₅)alkenyl;
V and X independently from each other represents a moiety selected from the group consisting of a bond, —CH₂—, —CH₂—CH₂—, —CH₂—CH₂—CH₂—, —CH₂—CH₂—CH₂—CH₂—, —CH₂—CH₂—CH₂—CH₂—CH₂—; or
V forms together with Ra and Rb and with the carbon they are attached to a cycloalkyl selected from the group consisting of adamantyl (C₁₀H₁₅), iceanyl (C₁₂H₁₇), diadamantanyl (C₁₄H₁₉), triadamantyl (C₂₂H₂₃), isotetramantanyl (C₂₂H₂₇), pentamantanyl (C₂₆H₃₁), cyclohexamantanyl (C₂₆H₂₉), superadamantan (C₃₀H₃₅); or
V forms together with Ra, Rb and with the carbon they are attached to a phenyl moiety which is substituted with —W—Re;
wherein W and Y independently from each other represents a moiety selected from the group consisting of *—O—, *—S—, *—NH—, *—N((C₁-C₅)alkyl), *-((L₁)-N)—C(O)-(L₂) wherein L₁ and L₂ are carbons of a lactam cycle of Rₑ or R_{f}, respectively, *—C(O)—O—, *—O—C(O)—, *—O—C(O)—NH—, *—S(O₂)—O—, *—O—S(O₂)—, *—C(S)—O—, *—O—C(S)—, *—C(O)—NH—, *—NH—C(O)—, *—S(O₂)—NH—, *—NH—S(O₂)—, *-triazol-, *—O—(C₂-C₅)alkyl-triazol-, *—O—C(O)—(C₂-C₅)alkyl-triazol-, *—C(O)O—(C₂-C₅)alkyl-triazol-, *-cyclohexenyl-, *—O-cyclohexenyl-, *—O—(C₁-C₅)alkyl-cyclohexenyl-, *—O—C(O)—(C₁-C₅)alkyl-cyclohexenyl-, *—O—C(O)-cyclohexenyl-, *—C(O)O—(C₁-C₅)alkyl-cyclohexenyl-, *—C(O)O-cyclohexenyl- wherein each cyclohexenyl moiety is optionally further substituted with 1, 2 or 3 (C₁-C₄)alkyl, 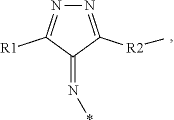
wherein R1 represents H or (C₁-C₅)alkyl and R₂ represents —(C₁-C₅)-alkyl-O— (bond to Re or Rf, respectively), —(C₁-C₅)-alkyl-O—C(O)— (bond to Re or Rf, respectively), —(C₁-C₅)-alkyl-C(O)O— (bond to Re or Rf, respectively),
wherein * indicates the bond to V and X, respectively, and wherein the other bond is a bond to a carbon (or in case of a lactam to two carbons) of Re or Rf, respectively;
Re is either a modified polymer or a modified drug,
wherein the modification in the modified Re is the replacement of a reactive group of the free form of Re wherein said reactive group is selected from the group consisting of —OH, —SH, —NH₂, —N((C₂-C₅)alkyl)H, L₁-N(H)—C(O)-L₂, wherein the amide function is part of a lactam of Re and L₁ and L₂ represent carbons of the lactam cycle, —C(O)OH, —C(O)NH₂, —S(O₂)OH, and a water soluble salt or anhydride of any of the foregoing, by W; or
wherein the modification is the replacement of a reactive group of the free form of a functionalized Re selected from the group consisting of —N₃, ethynyl, ethenyl, a conjugated diene, a tetrazine moiety and isonitrile,
by W and wherein W represents in such a case a moiety selected from the group consisting of *-triazol-, *—O—(C₂-C₅)alkyl-triazol-, *—O—C(O)—(C₂-C₅)alkyl-triazol-, *—C(O)O—(C₂-C₅)alkyl-triazol-, *-cyclohexenyl-, *—O—(C₂-C₅)alkyl-cyclohexenyl-, *—O—C(O)—(C₂-C₅)alkyl-cyclohexenyl-,*—C(O)O—(C₂-C₅)alkyl-cyclohexenyl-,*—S—CH₂—C₂—, 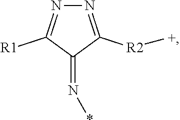
wherein R1 represents H or (C₁-C₅)alkyl and R₂ represents —(C₁-C₅)-alkyl-O— (bond to Re), —(C₁-C₅)-alkyl-O—C(O)— (bond to Re), —(C₁-C₅)-alkyl-C(O)O— (bond to Re),
wherein * indicates the bond to V, and wherein the other bond is a bond to a carbon of Re; and
Rf represents a modified drug
wherein the modification in the modified Rf is the replacement of a reactive group of the free form of Rf wherein said reactive group is selected from the group consisting of —OH, —SH, —NH₂, —N((C₂-C₅)alkyl)H, L₁-N(H)—C(O)-L₂, wherein the amide function is part of a lactam of Rf and L₁ and L₂ represent carbons of the lactam cycle, —C(O)OH, —C(O)NH₂, —S(O₂)OH, and a water soluble salt or anhydride of any of the foregoing,
by Y; or
wherein the modification is the replacement of a reactive group of the free form of a functionalized Rf selected from the group consisting of —N₃, ethynyl, ethenyl, a conjugated diene, a tetrazine moiety and isonitrile, preferably a reactive group is selected from the group consisting of —N₃ and -ethynyl,
by Y and wherein Y represents in such a case a moiety selected from the group consisting of *-triazol-, *—O—(C₂-C₅)alkyl-triazol-, *—O—C(O)—(C₂-C₅)alkyl-triazol-, *—C(O)O—(C₂-C₅)alkyl-triazol-, *-cyclohexenyl-, *—O—(C₂-C₅)alkyl-cyclohexenyl-, *—O—C(O)—(C₂-C₅)alkyl-cyclohexenyl-,*—C(O)O—(C₂-C₅)alkyl-cyclohexenyl-, and *—S—CH₂—C₂— 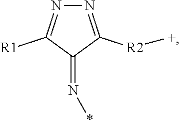
wherein R1 represents H or (C₁-C₅)alkyl and R₂ represents —(C₁-C₅)-alkyl-O— (bond to Rf), —(C₁-C₅)-alkyl-O—C(O)— (bond to Rf), —(C₁-C₅)-alkyl-C(O)O— (bond to Rf),
preferably the moiety is selected from the group consisting of *-triazol-, *—O—(C₂-C₅)alkyl-triazol-, *—O—C(O)—(C₂-C₅)alkyl-triazol-, *—C(O)O—(C₂-C₅)alkyl-triazol-, wherein * indicates the bond to X, and wherein the other bond is a bond to a carbon of Rf; and
optionally, Re may be further modified by at least one further substitution of a further reactive group of such a modified drug or polymer by a linker/Rf residue of formula (B) 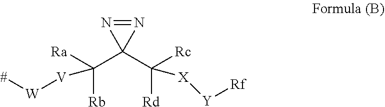
wherein # represents the bond to the further modified Re; and/or
Rf may be further modified by at least one further substitution of a further reactive group of such a modified drug by a linker/Rf residue of formula (C) 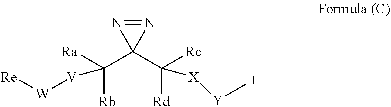
wherein + represents the bond to the further modified Rf.

2. The drug conjugate according to claim 1, wherein the conjugate is a conjugate of Formula (A2) 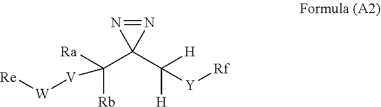
wherein Re, Rf, W, V, Y, Ra and Rb are as defined in Formula (A1).

3. A drug conjugate of formula (A1′) 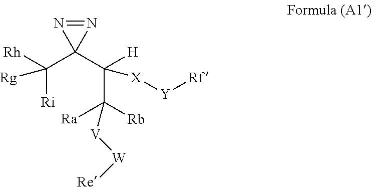
wherein V, W, X, Y, Ra and Rb are as defined for formula (A1) in claim 1 or claim 2;
Rh, Rg and Ri each individually represent halogen, preferably F, I or Cl, (C₁-C₅)alkyl, phenyl, benzyl; or
Rh, Rg and Ri form together with the carbon to which they are attached an adamantly moiety;
Re′ and Rf′ are identical to Re and Rf as defined for formula (A1) with the exception that an optionally further modified Re′ refers to a modification of Re′ in a conjugate of formula (A1′) with at least one further linker/Rf′ residue of formula (B′) and an optionally further modified Rf′ refers to a modification of Rf′ in a conjugate of formula (A1′) with at least one further linker/Re′ residue of formula (C′):
optionally, Re′ in Formula (A1′) may be further modified by at least one further substitution of a further reactive group of such a modified drug by a linker/Rf′ residue of formula (B′) 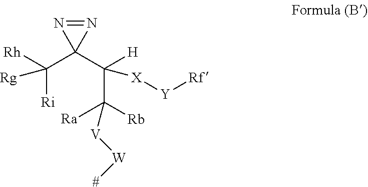
wherein # represents the bond to the further modified Re′; and/or
Rf′ may be further modified by at least one further substitution of a further reactive group of such a drug, by a linker/Re′ residue of formula (C′) 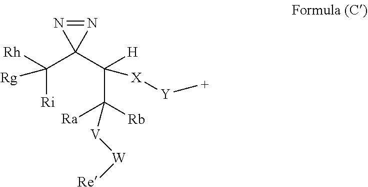
wherein + represents the bond to the further modified Rf′.

4. The drug conjugate according to anyone of the preceding claims, wherein Re or Re′ is a modified polymer.

5. The drug conjugate according to claim 4, wherein Re is further modified by at least one further substitution of a further reactive group of said polymer by a linker/Rf residue of formula (B) 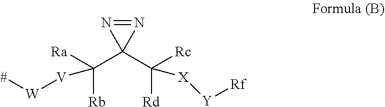
wherein # represents the bond to the further modified Re; or
Re′ is further modified by at least one further substitution of a further reactive group of said polymer by a linker/Rf′ residue of formula (B′) 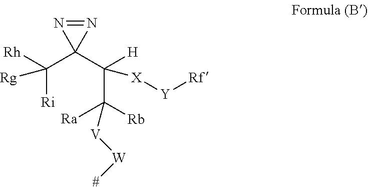
wherein # represents the bond to the further modified Re′.

6. The drug conjugate according to any of the preceding claims, wherein Re or Re′ is a modified polymer selected from the group consisting of polyacrylates (PA), polymethacrylates, polyacrylamides, polymethacrylamides, poly(alpha-hydroxy acids), poly(lactide-co-glycolides) (PLGA), polylactides (PLA), polyglycolides (PG), functionalized polystyrenes, polyethylene glycol (PEG), poly(alpha-hydroxy acids), polyorthoesters (POE), N-vinyl pyrrolidone, polyaspirins, polyphosphagenes, dendrimers, polyamides, proteins, peptides, polysaccharides.

7. The drug conjugate according to any one of the preceding claims, wherein V and X represent independently from each other a bond, —CH₂—, —CH₂—CH₂—, or —CH₂—CH₂—CH₂—.

8. The drug conjugate according to any one of the preceding claims, wherein W and Y independently from each other represents a moiety selected from the group consisting of *—O—, *—S—, *—NH—, *—O—C(O)—, *—NH—C(O)—, *—O—, *—S—, *—NH—, *—C(O)—O—, *—O—C(O)—, *—C(O)—NH—, *—NH—C(O)—, *-triazol-, —O—(C₂-C₅)alkyl-triazol-, *—O—C(O)—(C₂-C₅)alkyl-triazol-, *—C(O)O—(C₂-C₅)alkyl-triazol-.

9. The drug conjugate according to claim 8, wherein W and Y each represent a different moiety.

10. The drug conjugate according to any one of the preceding claims, wherein Rf represents a drug selected from the group consisting of ACE-inhibitors; anti-anginal drugs; anti-arrhythmias; anti-asthmatics; anti-cholesterolemics; anti-convulsants; anti-depressants; anti-diarrhea preparations; anti-histamines; anti-hypertensive drugs; anti-infectives; anti-inflammatory agents; anti-lipid agents; anti-manics; anti-nauseants; anti-stroke agents; anti-thyroid preparations; anti-tumor drugs; anti-tussives; anti-uricemic drugs; anti-viral agents; acne drugs; alkaloids; amino acid preparations; anabolic drugs; analgesics; anesthetics; angiogenesis inhibitors; antacids; anti-arthritics; antibiotics; anticoagulants; antiemetics; antiobesity drugs; antiparasitics; antipsychotics; antipyretics; antispasmodics; antithrombotic drugs; anxiolytic agents; appetite stimulants; appetite suppressants; beta blocking agents; bronchodilators; cardiovascular agents; cerebral dilators; chelating agents; cholecystokinin antagonists; chemotherapeutic agents; cognition activators; contraceptives; coronary dilators; cough suppressants; decongestants; deodorants; dermatological agents; diabetes agents; diuretics; emollients; enzymes; erythropoietic drugs; expectorants; fertility agents; fungicides; gastrointestinal agents; growth regulators; hormone replacement agents; hyperglycemic agents; hypnotics; hypoglycemic agents; laxatives; migraine treatments; mineral supplements; mucolytics; narcotics; neuroleptics; neuromuscular drugs; NSAIDS; nutritional additives; peripheral vasodilators; peptides; prostaglandins; psychotropics; renin inhibitors; respiratory stimulants; steroids; stimulants; sympatholytics; thyroid preparations; tranquilizers; uterine relaxants; vaginal preparations; vasoconstrictors; vasodilators; vertigo agents; vitamins; wound healing agents; and contrast agents.

11. The conjugate according to claim 10, wherein said drug is
a) a therapeutic peptide, an emergency drug, a cytotoxic agent, or an antibody; or b) a therapeutic peptide, preferably said therapeutic peptide is glucagon, GLP-1, or insulin, more preferably said therapeutic peptide is insulin, most preferably said therapeutic peptide is insulin in a hexameric form combined with zinc

12. A drug conjugate for use in medicine, wherein the drug conjugate comprises a diazirine group for releasing an immobilized drug from a drug conjugate by applying UV light to the diazirine group.

13. A linker molecule of formula (A′) 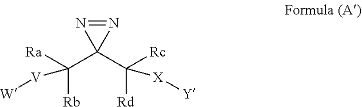
wherein
Ra and Rb independently from each other represents a moiety selected from halogen (preferably F, Cl, I or Br), (C₁-C₅)alkyl, (C₂-C₅)alkenyl, a five- or six-membered heterocycle, (C₃₋₃₀)cycloalkyl (preferably cyclopropyl, cyclopentyl, cyclohexanyl, adamantyl (C₁₀H₁₅), iceanyl (C₁₂H₁₇), diadamantanyl (C₁₄H₁₉), triadamantyl (C₂₂H₂₃), isotetramantanyl (C₂₂H₂₇), pentamantanyl (C₂₆H₃₁), cyclohexamantanyl (C₂₆H₂₉), superadamantan (C₃₀H₃₅)), phenyl, benzyl (—CH₂—C₆H₅) each heterocycle, cycloalkyl, phenyl or benzyl can optionally be substituted with 1, 2, 3, 4 or 5 substituents selected from the group consisting of hydroxy, halogen, (C₁-C₅)alkyl, (C₁-C₅)alkoxy; or
Ra and Rb form together with the carbon they are attached to a five- or six-membered heterocycle or (C₃₋₁₂)cycloalkyl, preferably cyclopentyl or cyclohexyl optionally substituted with 1, 2, 3, 4 or 5 substituents selected from the group consisting of hydroxy, halogen, (C₁-C₅)alkyl, (C₁-C₅)alkoxy); or
Ra and Rb represent together with the carbon they are attached to and V a (C₃₋₃₀)cycloalkyl moiety to which —W—Re is attached wherein the cycloalkyl moiety is selected from the group consisting of adamantyl (C₁₀H₁₄), iceanyl (C₁₂H₁₆), diadamantanyl (C₁₄H₁₈), triadamantyl (C₂₂H₂₂), isotetramantanyl (C₂₂H₂₆), pentamantanyl (C₂₆H₃₀), cyclohexamantanyl (C₂₆H₂₈), superadamantan (C₃₀H₃₄), each optionally substituted with 1, 2, 3, 4 or 5 substituents selected from the group consisting of hydroxy, halogen, (C₁-C₅)alkyl, (C₁-C₅)alkoxy; more preferably the cycloalkyl is selected from the group consisting of adamantyl (C₁₀H₁₄), iceanyl (C₁₂H₁₆), diadamantanyl (C₁₄H₁₈), triadamantyl (C₂₂H₂₂), isotetramantanyl (C₂₂H₂₆), pentamantanyl (C₂₆H₃₀), cyclohexamantanyl (C₂₆H₂₈), superadamantan (C₃₀H₃₄), more preferably the cycloalkyl is selected from the group consisting of adamantyl (C₁₀H₁₄), iceanyl (C₁₂H₁₆), diadamantanyl (C₁₄H₁₈), even more preferably adamantyl (C₁₀H₁₄)); or
Ra and Rb form together with the carbon they are attached to and V a phenyl moiety to which —W′ is attached;
Rc or Rd in formula (A′) represent H, the other space holder Rc or Rd, respectively, V and X are as defined in claims 1, 2 and 7 for the drug conjugate of formula (A1) and
W′ and Y′ are independently selected from a group consisting of
a) hydroxy (—OH), thiol (—SH), amin (—NH₂), —Cl, —Br, —F or —I, a cyclic amide function (—N(H)—C(O)—) such as in a lactame, wherein the free bond of the nitrogen and the free bond of the C(O) carbon each represents a bond to a neighbored carbon atom of the cycle, sulfonic acid (—S(O₂)OH), —O—S(O₂)OH, thiocarboxylic acid (—C(S)OH), carboxyl (carboxylic acid (—C(O)OH)), a water soluble salt, such as a pharmaceutically acceptable salt, thereof, or
b) a carboxylic acid halide and anhydrates thereof), amide (—C(O)NH₂), carboxylic acid ester, preferably with (C₁-C₅)alkyl, isothiocyanate (—NCS), isocyanate (—NCO) such as —(C₁-C₅)—NCO, —NCO, —O—(C₁-C₅)—NCO, —O—NCO, —O—C(O)—(C₁-C₅)—NCO, —O—C(O)—NCO, —C(O)O—(C₁-C₅)NCO or —C(O)O—NCO, hydrazine, a water soluble salt, preferably a pharmaceutically acceptable salt, of any of the foregoing, or c) alkyne such as —O—(C₁-C₅)alkyl-ethynyl, —C(O)O—(C₁-C₅)alkyl-ethynyl or —O—C(O)—(C₁-C₅)alkyl-ethynyl, azide, such as —(C₁-C₅)alkyl-N₃, —N₃, —O—(C₁-C₅)alkyl-N₃, —O—C(O)—(C₁-C₅)alkyl-N₃ or —C(O)O—(C₁-C₅)alkyl-N₃, maleimide, imidate, alkenyl such as —O—(C₃—C)-alkenyl, —C(O)—O—(C₃—C)-alkenyl, —O—C(O)—(C₃—C)-alkenyl, a conjugated diene such as a conjugated —(C₄-C₁₀)alkdienyl, a conjugated —O—(C₅-C₁₀)alkdienyl, a conjugated —O—C(O)—(C₅-C₁₀)alkdienyl or —C(O)O—(C₅C₁₀)alkdienyl, tetrazinyl such as —(C₁-C₅)alkyl-tetrazinyl, -tetrazinyl, —O—(C₁-C₅)tetrazinyl, —O-tetrazinyl, —O—C(O)—(C₁-C₅)tetrazinyl, —O—C(O)-tetrazinyl or —C(O)O—(C₀-C₅)tetrazinyl.

14. A depot suitable for implantation into a patient comprising at least one drug conjugate according to any one of claims 1 to 9.

15. A drug conjugate according to any one of claims 1 to 9 or the depot according to claim 14 for use in a method of administering a drug to a patient comprising: implanting the drug conjugate according to any one of claims 1 to 9 or the depot according to claim 14 into a patient; activating a UV light source to emit light with a wave length and intensity sufficient to cleave at least part of the linker of a conjugate according to any one of claims 1 to 9 thereby releasing the drug from the drug conjugate.
